(19) United States
(12) Patent Application Publication
(10) Pub. No.: US 2004/0077601 A1
(43) Pub. Date: Apr. 22, 2004

(54) Methods and compositions relating to isoleucine boroproline compounds

(75) Inventors: Sharlene Adams, Waltham, MA (US); Glenn T. Miller, Merrimac, MA (US); Michael I. Jesson, Hopedale, MA (US); Barry Jones, Cambridge, MA (US)

Correspondence Address: Maria A. Trevisan, Wolf, Greenfield & Sacks, P.C., 600 Atlantic Avenue, Boston, MA, 02210, US

(73) Assignee: Point Therapeutics, Inc., Boston, MA

(21) Appl. No.: 10/616,694

(22) Filed: Jul. 9, 2003

Related U.S. Application Data
(60) Provisional application No. 60/394,856, filed on Jul. 9, 2002.
(60) Provisional application No. 60/414,978, filed on Oct. 1, 2002.
(60) Provisional application No. 60/466,435, filed on Apr. 28, 2003.

(51) Int. Cl. A61K 31/69

(52) U.S. Cl. 514/64

(57) ABSTRACT

A method for treating subjects with, inter alia, abnormal cell proliferation or infectious disease. Compositions containing Ile-boroPro compounds are also provided. The invention embraces the use of these compounds alone or in combination with other therapeutic agents.

### RELATED APPLICATIONS

This application claims priority under 35 U.S.C. § 119(e) to U.S. Provisional Application Serial No. 60/394,856, entitled “BOROPROLINE COMPOUND COMBINATION THERAPY”, filed on Jul. 9, 2002; U.S. Provisional Application Serial No. 60/414,978, entitled “BOROPROLINE COMPOUND COMBINATION THERAPY”, filed on Oct. 1, 2002; and U.S. Provisional Application Serial No. 60/466,435, entitled “BOROPROLINE COMPOUND COMBINATION THERAPY”, filed on Apr. 28, 2003, each of which is herein incorporated by reference in its entirety.

### FIELD OF THE INVENTION

This invention relates, inter alia, to methods for the treatment of abnormal proliferative disorders and infectious diseases using isoleucine boroproline compounds.

### BACKGROUND OF THE INVENTION

Cancer is the second leading cause of death, resulting in one out of every four deaths, in the United States. In 1997, the estimated total number of new diagnoses for lung, breast, prostate, colorectal and ovarian cancer was approximately two million. Due to the ever increasing aging population in the United States, it is reasonable to expect that rates of cancer incidence will continue to grow.

Cancer is currently treated using a variety of modalities including surgery, radiation therapy and chemotherapy. The choice of treatment modality will depend upon the type, location and dissemination of the cancer. One of the advantages of surgery and radiation therapy is the ability to control to some extent the impact of the therapy, and thus to limit the toxicity to normal tissues in the body. Chemotherapy is arguably the most appropriate treatment for disseminated cancers such as leukemia and lymphoma as well as metastases. Chemotherapy is generally administered systemically and thus toxicity to normal tissues is a major concern. Not all tumors, however, respond to chemotherapeutic agents and others, although initially responsive to chemotherapeutic agents, may develop resistance. As a result, the search for effective anti-cancer drugs has intensified in an effort to find even more effective agents with less non-specific toxicity.

Recently, much emphasis has been placed on the use of immunotherapy for the treatment and prevention of cancer and other disorders, including infectious disease. Immunotherapy provides the cell specificity that other treatment modalities lack. Methods for enhancing the efficacy of immune based therapies would be beneficial.

### SUMMARY OF THE INVENTION

The invention provides methods and compositions relating to the treatment (including prevention) of inter alia disorders characterized by abnormal cell proliferation (e.g., cancer) and infectious diseases. The invention is based, in part, on the observation that agents of Formula I are particularly suited to treatment of these disorders when administered by injection or orally via an enterically coated form. Both forms of administration result in greater therapeutic efficacy for the agents of Formula I as compared to oral delivery. Administration by either of these latter routes avoids exposure of the agents of Formula I with an aminopeptidase present in the upper gastrointestinal tract (including stomach) that is suspected of degraded these agents specifically.

The agents of the invention share the common structure of Formula I:

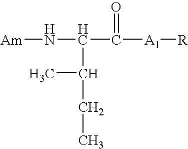

wherein Am and A₁ are L- or D-amino acids, m is an integer between 0 and 10, inclusive; A may be an L- or D-amino acid residue (except that for glycine there is no such distinction) such that each A in Aₘ may be an amino acid residue different from another or all other A in Aₘ; A₁ is bonded to the R with a C bond that is in the L-configuration. By “A₁ is bonded to the R with a C bond that is in the L-configuration” is meant that the absolute configuration of A₁ is like that of an L-amino acid. The R group can be organo boronates, organo phosphonates, fluoroalkylketones, alphaketos, N-peptiolyl-O-(acylhydroxylamines), azapeptides, azetidines, fluoroolefins dipeptide isoesteres, peptidyl (alpha-aminoalkyl) phosphonate esters, aminoacyl pyrrolidine-2-nitriles and 4-cyanothiazolidides, provided that it is capable of reacting with a functional group in the reactive site of FAP-α or other post proline-cleaving enzyme. Post proline-cleaving enzymes are enzymes which have a specificity for removing Xaa-Pro or Xaa-Ala dipeptides (where Xaa represents any amino acid) from the amino terminus of polypeptides. Examples of post-proline cleaving enzymes include, but are not limited to, CD26 and dipeptidyl peptidase IV (DP IV).

In certain embodiments, the agent may be 30, 20, 10 or less than 10 residues in length.

In one embodiment, the agent is also an agent of Formula II:

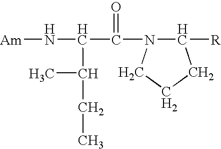

wherein Am is an L- or D-amino acid, m is an integer between 0 and 10, inclusive; A may be an L- or D-amino acid residue (except that for glycine there is no such distinction) such that each A in Aₘ may be an amino acid residue different from another or all other A in Aₘ; the C bonded to R is in the L-configuration. By “the C bonded to R is in the L-configuration” is meant that the absolute configuration of the C is like that of an L-amino acid. The R group can be organo boronates, organo phosphonates, fluoroalkylketones, alphaketos, N-peptiolyl-O-(acylhydroxylamines), azapeptides, azetidines, fluoroolefins dipeptide isoesteres, peptidyl (alpha-aminoalkyl) phosphonate esters, aminoacyl pyrrolidine-2-nitriles and 4-cyanothiazolidides, provided that it is capable of reacting with a functional group in the reactive site of FAP-α or other post proline-cleaving enzyme.

And in yet another embodiment, the agent is also an agent of Formula III:

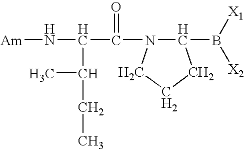

wherein Am is an L- or D-amino acid, m is an integer between 0 and 10, inclusive; A may be an L- or D-amino acid residue (except that for glycine there is no such distinction) such that each A in Aₘ may be an amino acid residue different from another or all other A in Aₘ; the C bonded to B is in the L-configuration; and each X₁ and X₂ is, independently, a hydroxyl group or a group capable of being hydrolyzed to a hydroxyl group in aqueous solution at physiological pH. By “the C bonded to B is in the L-configuration” is meant that the absolute configuration of the C is like that of an L-amino acid.

Thus, the

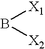

group has the same relationship to the C as the —COOH group of an L-amino acid has to its a carbon. In various embodiments, m is 0; X₁ and X₂ are hydroxyl groups; the inhibitor is Ile-boroPro. In some embodiments, the inhibitor is Ile-L-boroPro. In still other embodiments, inhibitor is L-Ile-L-boroPro.

In an important embodiment, the amino acids of these formulae are naturally occurring amino acids. In other embodiments, the amino acids of these formulae are non-naturally occurring or a mixture thereof.

In some embodiments, the agent is L-Ala-L-Ile-L-boroPro, L-Asp-L-Ile-L-boroPro, L-Glu-L-Ile-L-boroPro, L-Asn-L-Ile-L-boroPro, L-Gln-L-Ile-L-boroPro, L-Lys-L-Ile-L-boroPro, L-Arg-L-Ile-L-boroPro, L-His-L-Ile-L-boroPro, L-Pro-L-Ile-L-boroPro, L-Thr-L-Ile-L-boroPro, L-Ser-L-Ile-L-boroPro, L-Cys-L-Ile-L-boroPro, L-Gly-L-Ile-L-boroPro, L-Tyr-L-Ile-L-boroPro, L-Trp-L-Ile-L-boroPro, L-Phe-L-Ile-L-boroPro, L-Leu-L-Ile-L-boroPro, L-Ile-L-Ile-L-boroPro, L-Met-L-Ile-L-boroPro, or L-Val-L-Ile-L-boroPro.

In addition to agents of Formula II, other agents useful in the invention include those in which the proline residue in Formula II is replaced with another amino acid residue such as, for example, alanine. As well, derivatives of Formula III in which the boronate group is replaced with a reactive group as described above are also useful in the invention.

In one embodiment, the agent of Formula I is an agent of Formula II. In another embodiment, the agent of Formula I is an agent of Formula III. In an important embodiment, the agent of Formula I is L-Ile-L-boroPro. In another embodiment, the agent of Formula I is in a cyclic form. In yet another embodiment, the agent of Formula III is at least 96% L-isomer (i.e., at least 96% of the carbon atoms bearing boron are of the L-configuration).

In various embodiments of the methods recited herein, the agent of Formula I is administered on a routine schedule.

In one aspect, the agent of Formula I is administered to a subject in need thereof in an amount effective to inhibit abnormal mammalian cell proliferation (or a condition characterized by such) and thereby inhibit the condition. The agent of Formula I is administered by injection or in an enterically coated form. In certain embodiments, the subjects are otherwise free of symptoms calling for hemopoietic stimulation and, in particular, are free of symptoms calling for treatment with a compound for stimulating an immune response. The subjects to be treated may not exhibit symptoms requiring hemopoietic stimulation and they may have normal or protective levels of hemopoietic cells or normal hemopoietic activity. Included are subjects who are HIV positive but who have normal hemopoietic activity. In another embodiment, the subject is HIV negative. In certain embodiments, the subjects are not myeloid or lymphoid suppressed or are not candidates for treatment with an agent which causes such suppression at the time of treatment with the methods of the invention.

The condition associated with abnormal mammalian cell proliferation may be a cancer such as those recited below, including a refractory cancer, or an immunogenic cancer. In some embodiments, the condition characterized by abnormal mammalian cell proliferation is further characterized by the presence of reactive stromal fibroblasts. The condition may also be a premalignant condition or a benign tumor. In one aspect, the invention provides a method for inhibiting angiogenesis, particularly when associated with abnormal mammalian cell proliferation. As disclosed herein, the agents of Formula I induce several angiogenic factors including thrombospondin, IP-10 and MIG.

In one embodiment, the cancer is selected from the group consisting of basal cell carcinoma, biliary tract cancer; bladder cancer; bone cancer; brain cancer; breast cancer; cervical cancer; choriocarcinoma; CNS cancer; colon and rectum cancer; connective tissue cancer; cancer of the digestive system; endometrial cancer; esophageal cancer; eye cancer; cancer of the head and neck; gastric cancer; intra-epithelial neoplasm; kidney cancer; larynx cancer; leukemia; acute myeloid leukemia, acute lymphoid leukemia, chronic myeloid leukemia, chronic lymphoid leukemia, liver cancer; small cell lung cancer; non-small cell lung cancer; lymphoma, Hodgkin's lymphoma; Non-Hodgkin's lymphoma; melanoma; myeloma; neuroblastoma; oral cavity cancer; ovarian cancer; pancreatic cancer; prostate cancer; retinoblastoma; rhabdomyosarcoma; rectal cancer; renal cancer; cancer of the respiratory system; sarcoma; skin cancer; stomach cancer; testicular cancer; thyroid cancer; uterine cancer; and cancer of the urinary system.

In another embodiment, the cancer is selected from the group consisting of bladder cancer, breast cancer, colon cancer, endometrial cancer, head and neck cancer, leukemia, lung cancer, lymphoma, melanoma, ovarian cancer, prostate cancer and rectal cancer. The cancer may be a carcinoma and a sarcoma, or a leukemia or a lymphoma. In still another embodiment, the cancer is a metastasis.

Thus, in another aspect, the subjects are treated with the agent of Formula I in a manner and in an amount so as to inhibit proliferation of a primary tumor, or to inhibit metastatic spread or growth while minimizing the potential for systemic toxicity.

In some embodiments of the invention, the agent is administered in combination with an anti-cancer therapy other than an agent of Formula I, such as an anti-cancer compound (chemotherapy), radiation or surgery. The agent may be administered either before, at the same time as, and/or after anti-proliferative therapy.

The chemotherapy may be selected from the group consisting of aldesleukin, asparaginase, bleomycin sulfate, carboplatin, chlorambucil, cisplatin, cladribine, cyclophosphamide, cytarabine, dacarbazine, dactinomycin, daunorubicin hydrochloride, docetaxel, doxorubicin, doxorubicin hydrochloride, epirubicin hydrochloride, etoposide, etoposide phosphate, floxuridine, fludarabine, fluorouracil, gemcitabine, gemcitabine hydrochloride, hydroxyurea, idarubicin hydrochloride, ifosfamide, interferons, interferon-α2a, interferon-α2b, interferon-αn3, interferon-α1b, interleukins, irinotecan, mechlorethamine hydrochloride, melphalan, mercatopurine, methotrexate, methotrexate sodium, mitomycin, mitoxantrone, paclitaxel, pegaspargase, pentostatin, prednisone, profimer sodium, procabazine hydrochloride, taxol, taxotere, teniposide, topotecan hydrochloride, vinblastine sulfate, vincristine sulfate or vinorelbine tartrate.

The agent of Formula may be administered prior to, during and/or after the anti-cancer therapy. For example, the agent of Formula I may be administered substantially simultaneously with the anti-cancer therapy. In one embodiment, the agent of Formula I is administered daily and the chemotherapy is administered weekly, biweekly, or every three weeks. The agent of Formula I may be also administered twice a day.

The invention provides a related method for treating a condition associated with abnormal angiogenesis comprising administering to a subject in need thereof an agent of Formula I in an amount effective to inhibit the condition. In a preferred embodiment, the condition is a tumor.

In yet another aspect, the invention provides a method for treating an infectious disease comprising administering to a subject in need thereof an agent of Formula I in an effective amount to inhibit the infectious disease, wherein the agent of Formula I is administered by injection or in an enterically coated form. The method may further comprise administering to the subject an anti-microbial agent which in turn may be an anti-bacterial agent, an anti-viral agent, an anti-fungal agent, an anti-parasitic agent or an anti-mycobacterial agent. The anti-microbial agent may be an anti-bacterial agent such as an antibiotic. The antibiotic may be a broad spectrum antibiotic, a narrow spectrum antibiotic, or a limited spectrum antibiotic. The anti-bacterial agent may also be a cell wall synthesis inhibitor, cell membrane inhibitor, protein synthesis inhibitor, nucleic acid synthesis or functional inhibitor or a competitive inhibitor.

Examples of suitable anti-bacterial agents include but are not limited to natural penicillins, semi-synthetic penicillins, clavulanic acid, cephalolsporins, bacitracin, ampicillin, carbenicillin, oxacillin, azlocillin, mezlocillin, piperacillin, methicillin, dicloxacillin, nafcillin, cephalothin, cephapirin, cephalexin, cefamandole, cefaclor, cefazolin, cefuroxine, cefoxitin, cefotaxime, cefsulodin, cefetamet, cefixime, ceftriaxone, cefoperazone, ceftazidine, moxalactam, carbapenems, imipenems, monobactems, euztreonam, vancomycin, polymyxin, amphotericin B, nystatin, imidazoles, clotrimazole, miconazole, ketoconazole, itraconazole, fluconazole, rifampins, ethambutol, tetracyclines, chloramphenicol, macrolides, aminoglycosides, streptomycin, kanamycin, tobramycin, amikacin, gentamicin, tetracycline, minocycline, doxycycine, chlortetracycline, erythromycin, roxithromycin, clarithromycin, oleandomycin, azithromycin, chloramphenicol, quinolones, co-trimoxazole, norfloxacin, ciprofloxacin, enoxacin, nalidixic acid, temafloxacin, sulfonamides, gantrisin, and trimethoprim.

Other suitable anti-bacterial agents include but are not limited to acedapsone; acetosulfone sodium; alamecin; alexidine; amdinocillin; amdinocillin pivoxil; amicycline; amifloxacin; amifloxacin mesylate; amikacin; amikacin sulfate; aminosalicylic acid; aminosalicylate sodium; amoxicillin; amphomycin; ampicillin; ampicillin sodium; apalcillin sodium; apramycin; aspartocin; astromicin sulfate; avilamycin; avoparcin; azithromycin; aziocillin; azlocillin sodium; bacampicillin hydrochloride; bacitracin; bacitracin methylene disalicylate; bacitracin zinc; bambermycins; benzoylpas calcium; berythromycin; betamicin sulfate; biapenem; biniramycin; biphenamine hydrochloride; bispyrithione magsulfex; butikacin; butirosin sulfate; capreomycin sulfate; carbadox; carbenicillin disodium; carbenicillin indanyl sodium; carbenicillin phenyl sodium; carbenicillin potassium; carumonam sodium; cefaclor; cefadroxil; cefamandole; cefamandole nafate; cefamandole sodium; cefaparole; cefatrizine; cefazaflur sodium; cefazolin; cefazolin sodium; cefbuperazone; cefdinir; cefepime; cefepime hydrochloride; cefetecol; cefixime; cefinenoxime hydrochloride; cefinetazole; cefinetazole sodium; cefonicid monosodium; cefonicid sodium; cefoperazone sodium; ceforanide; cefotaxime sodium; cefotetan; cefotetan disodium; cefotiam hydrochloride; cefoxitin; cefoxitin sodium; cefpimizole; cefpimizole sodium; cefpiramide; cefpiramide sodium; cefpirome sulfate; cefpodoxime proxetil; cefprozil; cefroxadine; cefsulodin sodium; ceftazidime; ceftibuten; ceftizoxime sodium; ceftriaxone sodium; cefuroxime; cefuroxime axetil; cefuroxime pivoxetil; cefuroxime sodium; cephacetrile sodium; cephalexin; cephalexin hydrochloride; cephaloglycin; cephaloridine; cephalothin sodium; cephapirin sodium; cephradine; cetocycline hydrochloride; cetophenicol; chloramphenicol; chloramphenicol palmitate; chloramphenicol pantothenate complex; chloramphenicol sodium succinate; chlorhexidine phosphanilate; chloroxylenol; chlortetracycline bisulfate; chlortetracycline hydrochloride; cinoxacin; ciprofloxacin; ciprofloxacin hydrochloride; cirolemycin; clarithromycin; clinafloxacin hydrochloride; clindamycin; clindamycin hydrochloride; clindamycin palmitate hydrochloride; clindamycin phosphate; clofazimine; cloxacillin benzathine; cloxacillin sodium; cloxyquin; colistimethate sodium; colistin sulfate; coumermycin; coumermycin sodium; cyclacillin; cycloserine; dalfopristin; dapsone; daptomycin; demeclocycline; demeclocycline hydrochloride; demecycine; denofungin; diaveridine; dicloxacilin; dicloxacillin sodium; dihydrostreptomycin sulfate; dipyrithione; dirithromycin; doxycycline; doxycycline calcium; doxycycline fosfatex; doxycycline hyclate; droxacin sodium; enoxacin; epicillin; epitetracycline hydrochloride; erythromycin; erythromycin acistrate; erythromycin estolate; erythromycin ethylsuccinate; erythromycin gluceptate; erythromycin lactobionate; erythromycin propionate; erythromycin stearate; ethambutol hydrochloride; ethionamide; fleroxacin; floxacillin; fludalanine; flumequine; fosfomycin; fosfomycin tromethamine; fumoxicillin; furazolium chloride; furazolium tartrate; fusidate sodium; fusidic acid; gentamicin sulfate; gloximonam; gramicidin; haloprogin; hetacillin; hetacillin potassium; hexedine; ibafloxacin; imipenem; isoconazole; isepamicin; isoniazid; josamycin; kanamycin sulfate; kitasamycin; levofuraltadone; levopropylcillin potassium; lexithromycin; lincomycin; lincomycin hydrochloride; lomefloxacin; lomefloxacin hydrochloride; lomefloxacin mesylate; loracarbef; mafenide; meclocycline; meclocycline sulfosalicylate; megalomicin potassium phosphate; mequidox; meropenem; methacycline; methacycline hydrochloride; methenamine; methenamine hippurate; methenamine mandelate; methicillin sodium; metioprim; metronidazole hydrochloride; metronidazole phosphate; meziocillin; meziocillin sodium; minocycline; minocycline hydrochloride; mirincamycin hydrochloride; monensin; monensin sodium; nafcillin sodium; nalidixate sodium; nalidixic acid; natamycin; nebramycin; neomycin palmitate; neomycin sulfate; neomycin undecylenate; netilmicin sulfate; neutramycin; nifuradene; nifuraldezone; nifuratel; nifuratrone; nifurdazil; nifurimide; nifurpirinol; nifurquinazol; nifurthiazole; nitrocycline; nitrofurantoin; nitromide; norfloxacin; novobiocin sodium; ofloxacin; ormetoprim; oxacillin sodium; oximonam; oximonam sodium; oxolinic acid; oxytetracycline; oxytetracycline calcium; oxytetracycline hydrochloride; paldimycin; parachlorophenol; paulomycin; pefloxacin; pefloxacin mesylate; penamecillin; penicillin g benzathine; penicillin g potassium; penicillin g procaine; penicillin g sodium; penicillin v; penicillin v benzathine; penicillin v hydrabamine; penicillin v potassium; pentizidone sodium; phenyl aminosalicylate; piperacillin sodium; pirbenicillin sodium; piridicillin sodium; pirlimycin hydrochloride; pivampicillin hydrochloride; pivampicillin pamoate; pivampicillin probenate; polymyxin b sulfate; porfiromycin; propikacin; pyrazinamide; pyrithione zinc; quindecamine acetate; quinupristin; racephenicol; ramoplanin; ranimycin; relomycin; repromicin; rifabutin; rifametane; rifamexil; rifamide; rifampin; rifapentine; rifaximin; rolitetracycline; rolitetracycline nitrate; rosaramicin; rosaramicin butyrate; rosaramicin propionate; rosaramicin sodium phosphate; rosaramicin stearate; rosoxacin; roxarsone; roxithromycin; sancycline; sanfetrinem sodium; sarmoxicillin; sarpicillin; scopafungin sisomicin; sisomicin sulfate; sparfloxacin; spectinomycin hydrochloride; spiramycin; stallimycin hydrochloride; steffimycin; streptomycin sulfate; streptonicozid; sulfabenz; sulfabenzamide; sulfacetamide; sulfacetamide sodium; sulfacytine; sulfadiazine; sulfadiazine sodium; sulfadoxine; sulfalene; sulfamerazine; sulfameter; sulfamethazine; sulfamethizole; sulfamethoxazole; sulfamonomethoxine; sulfamoxole; sulfanilate zinc; sulfanitran; sulfasalazine; sulfasomizole; sulfathiazole; sulfazamet; sulfisoxazole; sulfisoxazole acetyl; sulfisoxazole diolamine; sulfomyxin; sulopenem; sultamicillin; suncillin sodium; talampicillin hydrochloride; teicoplanin; temafloxacin hydrochloride; temocillin; tetracycline; tetracycline hydrochloride; tetracycline phosphate complex; tetroxoprim; thiamphenicol; thiphencillin potassium; ticarcillin cresyl sodium; ticarcillin disodium; ticarcillin monosodium; ticlatone; tiodonium chloride; tobramycin; tobramycin sulfate; tosufloxacin; trimethoprim; trimethoprim sulfate; trisulfapyrimidines; troleandomycin; trospectomycin sulfate; tyrothricin; vancomycin; vancomycin hydrochloride; virginiamycin; and zorbamycin.

In another embodiment, the anti-microbial agent is an anti-viral agent which may be selected from the group consisting of immunoglobulin, amantadine, interferon, nucleoside analogue, nonnucleoside analogue, biflavanoid and protease inhibitor, although it is not so limited. In one embodiment, the protease inhibitor is indinavir, saquinavir, ritonavir, and nelfinavir. In another embodiment, the biflavanoid is robustaflavone, amentoflavone, or a derivative or salt thereof. In yet another embodiment, the non-nucleoside analogue is selected from the group consisting of delavirdine, nevirapine, efavirenz, alpha-interferon, recombinant CD4, amantadine, rimantadine, ribavirin and vidarabine.

Examples of suitable antiviral agent include but are not limited to AZT, ddC, ddI, D4T, 3TC, acemannan; acyclovir; acyclovir sodium; adefovir; alovudine; alvircept sudotox; amantadine hydrochloride; aranotin; arildone; atevirdine mesylate; pyridine; cidofovir; cipamfylline; cytarabine hydrochloride; delavirdine mesylate; desciclovir; didanosine; disoxaril; edoxudine; enviradene; enviroxime; famciclovir; famotine hydrochloride; fiacitabine; fialuridine; fluorinated nucleosides; fosarilate; foscarnet sodium; fosfonet sodium; ganciclovir; ganciclovir sodium; idoxuridine; kethoxal; lamivudine; lobucavir; memotine hydrochloride; methisazone; nevirapine; penciclovir; pirodavir; ribavirin; rimantadine hydrochloride; saquinavir mesylate; somantadine hydrochloride; sorivudine; statolon; stavudine; tilorone hydrochloride; trifluridine; valacyclovir hydrochloride; vidarabine; vidarabine phosphate; vidarabine sodium phosphate; viroxime; zalcitabine; zidovudine; and zinviroxime.

The anti-microbial agent may also be an anti-fungal agent such as but not limited to imidazole, FK 463, amphotericin B, BAY 38-9502, MK 991, pradimicin, UK 292, butenafine, chitinase and 501 cream.

Examples of suitable anti-fungal agents include but are not limited to acrisorcin; ambruticin; amorolfine, amphotericin b; azaconazole; azaserine; basifungin; bifonazole; biphenamine hydrochloride bispyrithione magsulfex; butoconazole nitrate; calcium undecylenate; candicidin; carbol-fuchsin; chlordantoin; ciclopirox; ciclopirox olamine; cilofungin; cisconazole; clotrimazole; cuprimyxin; denofungin; dipyrithione; doconazole; econazole; econazole nitrate; enilconazole; ethonam nitrate; fenticonazole nitrate; filipin; fluconazole; flucytosine; fungimycin; griseofulvin; hamycin; isoconazole; itraconazole; kalafungin; ketoconazole; lomofungin; lydimycin; mepartricin; iniconazole; miconazole nitrate; monensin; monensin sodium; naftifine hydrochloride; neomycin undecylenate; nifuratel; nifurmerone; nitralamine hydrochloride; nystatin; octanoic acid; orconazole nitrate; oxiconazole nitrate; oxifungin hydrochloride; parconazole hydrochloride; partricin; potassium iodide; proclonol; pyrithione zinc; pyrrolnitrin; rutamycin; sanguinarium chloride; saperconazole; scopafungin; selenium sulfide; sinefungin; sulconazole nitrate; terbinafine; terconazole; thiram; ticlatone; tioconazole; tolciclate; tolindate; tolnaftate; triacetin; triafungin; undecylenic acid; viridofulvin; zinc undecylenate; and zinoconazole hydrochloride.

The anti-microbial agent may also be an anti-parasitic agent. Examples of suitable anti-parasitic agents include but are not limited to albendazole, amphotericin B, benznidazole, bithionol, chloroquine HCl, chloroquine phosphate, clindamycin, dehydroemetine, diethylcarbamazine, diloxanide furoate, eflornithine, furazolidaone, glucocorticoids, halofantrine, iodoquinol, ivermectin, mebendazole, mefloquine, meglumine antimoniate, melarsoprol, metrifonate, metronidazole, niclosamide, nifurtimox, oxamniquine, paromomycin, pentamidine isethionate, piperazine, praziquantel, primaquine phosphate, proguanil, pyrantel pamoate, pyrimethanmine-sulfonamides, pyrimethanmine-sulfadoxine, quinacrine HCl, quinine sulfate, quinidine gluconate, spiramycin, stibogluconate sodium (sodium antimony gluconate), suramin, tetracycline, doxycycline, thiabendazole, tinidazole, trimethroprim-sulfamethoxazole, and tryparsamide.

The anti-microbial agent may also be an anti-mycobacterial agent, such as but not limited to an anti-tuberculosis agent. The anti-tuberculosis agent may be isoniazid, rifampin, rifabutin, rifapentine, pyrazinamide, ethambutol, (+)calanolide A, (−)-calanolide A, (−)-soulattrolide, (−)-costatolide or (−)-7,8-dihydrosoulattrolide. Other anti-mycobacterial agent include streptomycin, dapsone, clarithromycin, ciprofloxacin, clofazamine, azithromycin, ethionamide, amikacin or resorcinomycin A.

In one embodiment, the agent of Formula I is administered prior to, substantially simultaneously with, and/or after the anti-microbial agent. The agent of Formula I may be provided as a pharmaceutical preparation prepared within thirty minutes of administration.

The invention is further premised on the finding that the agents of Formula I stimulate the production of IL-1α, IL-1β, MCP-2, MARC/MCP-3, MCP-5, JE, G-CSF, MIP-2, IL-8 (KC in mouse), ENA78, LIX, lymphotactin, eotaxin, IL-6, MIG, IP-10, MDC, TARC, and thrombospondin, among others. Some of these cytokines activate macrophages and other antigen presenting cells, and thus are useful in enhancing immune responses including antibody dependent cell-mediated cytotoxicity and antigen presentation.

The ability of these compounds to stimulate cytokine and chemokine production endogenously is beneficial since exogenous administration of some of these factors, such as for example IL-1, has been associated with toxicity. Production of IL-1 endogenously, and particularly in the spleen and lymph nodes, with no detection in the serum indicates that the agents of Formula I can be used to induce cytokines in a controlled manner, and thereby overcome toxicity problems. Although not intending to be bound by any particular mechanism, it is further proposed that induction of these cytokines in vivo also indicates that feedback loops normally operating in vivo may be operative and can control cytokine levels.

The invention is therefore also based in part on the observation that compounds of Formula I can be administered with disease specific antibodies in order to enhance the efficacy of such antibodies. Again, although not intending to be bound by any particular mechanism, it is proposed that the production of cytokines following administration of Formula I compounds leads to the stimulation of immune cells, thereby enhancing the response mediated by the exogenously administered antibody. Also again, the agents of Formula I are administered in these methods either by injection or using an enterically coated form.

The invention relates to methods and compositions for enhancing immune therapies for a number of indications, both in a therapeutic and a prophylactic sense. Immune therapies include but are not limited to passive immune therapies such as immunoglobulin administration, and active immune therapies such as vaccination with antigens alone or antigens in the context of dendritic cells. The methods are intended to treat or prevent various indications that would benefit from an enhanced immune response.

In important aspects of the invention, the agents of Formula I are administered with an antibody or antibody fragment, with an antigen and optionally with an adjuvant, or as stand alone compositions. In some embodiments, the immune response that is stimulated is a cell-mediated immune response involving T cells, NK cells, macrophages, and the like. In other embodiments, the immune response that is stimulated is a humoral response involving B cells and antibody production. Both types of responses can co-exist in yet other embodiments. In still other embodiments, the immune response is an innate immune response, while in others it is an adaptive immune response. The immune response therefore may be antigen specific. It may also involve antibody-dependent cell cytotoxicity (ADCC).

Depending upon the aspect of the invention, the subject may be one in need of immune stimulation is a subject having or at risk of developing cancer such as those recited herein. The subject in need of immune stimulation may also be a subject having or at risk of developing an infectious disease. As used herein, the terms “infectious disease” and “microbial infection” are used interchangeably and intended to convey an infection by any microbe including but not limited to a bacterium, a mycobacterium, a virus, a fungus, a parasite, and the like. Thus, the infectious disease may be selected from the group consisting of a bacterial infection, a mycobacterial infection, a viral infection, a fungal infection and a parasitic infection, but it is not so limited.

In one embodiment, the bacterial infection is selected from the group consisting of an E. coli infection, a Staphylococcal infection, a Streptococcal infection, a Pseudomonas infection, Clostridium difficile infection, Legionella infection, Pneumococcus infection, Haemophilus infection, Klebsiella infection, Enterobacter infection, Citrobacter infection, Neisseria infection, Shigella infection, Salmonella infection, Listeria infection, Pasteurella infection, Streptobacillus infection, Spirillum infection, Treponema infection, Actinoinyces infection, Borrelia infection, Corynebacterium infection, Nocardia infection, Gardnerella infection, Campylobacter infection, Spirochaeta infection, Proteus infection, Bacteriodes infection, H. pylori infection, and anthrax infection.

The mycobacterial infection may be tuberculosis or leprosy respectively caused by the M. tuberculosis and M. leprae species, but is not so limited.

In one embodiment, the viral infection is selected from the group consisting of an HIV infection, a Herpes simplex virus 1 infection, a Herpes simplex virus 2 infection, cytomegalovirus infection, hepatitis A virus infection, hepatitis B virus infection, hepatitis C virus infection, human papilloma virus infection, Epstein Barr virus infection, rotavirus infection, adenovirus infection, influenza A virus infection, respiratory syncytial virus infection, varicella-zoster virus infections, small pox infection, monkey pox infection, and SARS infection. In some embodiments, the viral infection is not an HIV infection.

In yet another embodiment, the fungal infection selected from the group consisting of candidiasis, ringworm, histoplasmosis, blastomycosis, paracoccidioidomycosis, crytococcosis, aspergillosis, chromomycosis, mycetoma infections, pseudallescheriasis, and tinea versicolor infection.

In another embodiment, the parasite infection is selected from the group consisting of amebiasis, Trypanosoma cruzi infection, Fascioliasis, Leishmaniasis, Plasmodium infections, Onchocerciasis, Paragonimiasis, Trypanosoma brucei infection, Pneumocystis infection, Trichomonas vaginalis infection, Taenia infection, Hymenolepsis infection, Echinococcus infections, Schistosomiasis, neurocysticercosis, Necator americanus infection, and Trichuris trichuria infection.

In one aspect, the invention provides a method for stimulating an immune response in a subject comprising administering to a subject in need of immune stimulation an agent of Formula I, and an antibody or antibody fragment, in an amount effective to stimulate an immune response. The agent of Formula I may be formulated with the antibody or antibody fragment.

The antibody or antibody fragment may be specific for a cell surface molecule. Cell surface molecules that may be targeted with the antibody or antibody fragment include but are not limited to HER 2, CD20, CD33, EGF receptor, HLA markers such as HLA-DR, CD52, CD1, CEA, CD22, GD2 ganglioside, FLK2/FLT3, VEGF, VEGFR, and the like.

The antibody or antibody fragment may be specific for a cancer antigen. Cancer antigens that may be targeted with the antibody or antibody fragment have been recited throughout the specification and include but are not limited to HER 2 (p185), CD20, CD33, GD3 ganglioside, GD2 ganglioside, carcinoembryonic antigen (CEA), CD22, milk mucin core protein, TAG-72, Lewis A antigen, ovarian associated antigens such as OV-TL3 and MOv18, high Mr melanoma antigens recognized by antibody 9.2.27, HMFG-2, SM-3, B72.3, PR5C5, PR4D2, and the like. Other cancer antigens are described in U.S. Pat. No. 5,776,427. Still other cancer antigens are recited herein in Table 1.

Cancer antigens can be classified in a variety of ways. Cancer antigens include antigens encoded by genes that have undergone chromosomal alteration. Many of these antigens are found in lymphoma and leukemia. Even within this classification, antigens can be characterized as those that involve activation of quiescent genes. These include BCL-1 and IgH (Mantel cell lymphoma), BCL-2 and IgH (Follicular lymphoma), BCL-6 (Diffuse large B-cell lymphoma), TAL-1 and TCRδ or SIL (T-cell acute lymphoblastic leukemia), c-MYC and IgH or IgL (Burkitt lymphoma), MUN/IRF4 and IgH (Myeloma), PAX-5 (BSAP) (Immunocytoma).

Other cancer antigens that involve chromosomal alteration and thereby create a novel fusion gene and/or protein include RARα, PML, PLZF, NPM or NuMA (Acute promyelocytic leukemia), BCR and ABL (Chronic myeloid/acute lymphoblastic leukemia), MLL (HRX) (Acute leukemia), E2A and PBX or HLF (B-cell acute lymphoblastic leukemia), NPM, ALK (Anaplastic large cell leukemia), and NPM, MLF-1 (Myelodysplastic syndrome/acute myeloid leukemia).

Other cancer antigens are specific to a tissue or cell lineage. These include cell surface proteins such as CD20, CD22 (Non-Hodgkin's lymphoma, B-cell lymphoma, chronic lymphocytic leukemia (CLL)), CD52 (B-cell CLL), CD33 (acute myelogenous leukemia (AML)), CD10 (gp100) (common (pre-B) acute lymphocytic leukemia and malignant melanoma), CD3/T-cell receptor (TCR) (T-cell lymphoma and leukemia), CD79/B-cell receptor (BCR) (B-cell lymphoma and leukemia), CD26 (epithelial and lymphoid malignancies), human leukocyte antigen (HLA)-DR, HLA-DP, and HLA-DQ (lymphoid malignancies), RCAS1 (gynecological carcinomas, bilary adenocarcinomas and ductal adenocarcinomas of the pancreas), and prostate specific membrane antigen (prostate cancer).

Tissue- or lineage-specific cancer antigens also include epidermal growth factor receptors (high expression) such as EGFR (HER1 or erbB1) and EGFRvIII (brain, lung, breast, prostate and stomach cancer), erbB2 (HER2 or HER2/neu) (breast cancer and gastric cancer), erbB3 (HER3) (adenocarcinoma), and erbB4 (HER4) (breast cancer).

Tissue- or lineage-specific cancer antigens also include cell-associated proteins such as tyrosinase, melan-A/MART-1, tyrosinase related protein (TRP)-1/gp75 (malignant melanoma), polymorphic epithelial mucin (PEM) (breast tumors), and human epithelial mucin (MUC1) (breast, ovarian, colon and lung cancers).

Tissue- or lineage-specific cancer antigens also include secreted proteins such as monoclonal immunoglobulin (multiple myeloma and plasmacytoma), immunoglobulin light chains (Multiple Myeloma), α-fetoprotein (liver carcinoma), kallikreins 6 and 10 (ovarian cancer), gastrin-releasing peptide/bombesin (lung carcinoma), and prostate specific antigen (prostate cancer).

Still other cancer antigens are cancer testis (CT) antigens that are expressed in some normal tissues such as testis and in some cases placenta. Their expression is common in tumors of diverse lineages and as a group the antigens form targets for immunotherapy. Examples of tumor expression of CT antigens include MAGE-A1, -A3, -A6, -A12, BAGE, GAGE, HAGE, LAGE-1, NY-ESO-1, RAGE, SSX-1, -2, -3, -4, -5, -6, -7, -8, -9, HOM-TES-14/SCP-1, HOM-TES-85 and PRAME. Still other examples of CT antigens and the cancers in which they are expressed include SSX-2, and -4 (Neuroblastoma), SSX-2 (HOM-MEL-40), MAGE, GAGE, BAGE and PRAME (Malignant melanoma), HOM-TES-14/SCP-1 (Meningioma), SSX-4 (Oligodendrioglioma), HOM-TES-14/SCP-1, MAGE-3 and SSX-4 (Astrocytoma), SSX member (Head and neck cancer, ovarian cancer, lymphoid tumors, colorectal cancer and breast cancer), RAGE-1, -2, -4, GAGE-1, -2, -3, -4, -5, -6, -7 and -8 (Head and neck squamous cell carcinoma (HNSCC)), HOM-TES14/SCP-1, PRAME, SSX-1 and CT-7 (Non-Hodgkin's lymphoma), and PRAME (Acute lymphoblastic leukemia (ALL), acute myelogenous leukemia (AML) and chronic lymphocytic leukemia (CLL)).

Other cancer antigens are not specific to a particular tissue or cell lineage. These include members of the carcinoembryonic antigen (CEA) family: CD66a, CD66b, CD66c, CD66d and CD66e. These antigens can be expressed in many different malignant tumors and can be targeted by immunotherapy. Still other cancer antigens are viral proteins and these include Human papilloma virus protein (cervical cancer), and EBV-encoded nuclear antigen (EBNA)-1 (lymphomas of the neck and oral cancer). Still other cancer antigens are mutated or aberrantly expressed molecules such as but not limited to CDK4 and beta-catenin (melanoma).

The invention embraces the use of antibodies or antibodies fragments specific for any of the foregoing cancer antigens.

The antibody or antibody fragment may be specific for a stromal cell molecule. Stromal cell molecules that may be targeted with the antibody or antibody fragment include but are not limited to FAP and CD26. The antibody or antibody fragment may be specific for an extracellular matrix molecule. Extracellular matrix molecules that may be targeted with the antibody or antibody fragment include but are not limited to collagen, glycosaminoglycans (GAGs), proteoglycans, elastin, fibronectin and laminin.

The antibody or antibody fragment may be specific for a tumor vasculature molecule. Tumor vasculature molecules include but are not limited to endoglin, ELAM-1, VCAM-1, ICAM-1, ligand reactive with LAM-1, MHC class II antigens, aminophospholipids such as phosphatidylserine and phosphatidylethanolamine, VEGFR1 (Flt-1) and VEGFR2 (KDR/Flk-1). Antibodies to endoglin include TEC-4 and TEC-11. Antibodies that inhibit VEGF include 2C3 (ATCC PTA 1595). Other antibodies that are specific for tumor vasculature include antibodies that react to a complex of a growth factor and its receptor such as a complex of FGF and the FGFR or a complex of TGFβ and the TGFβR. Antibodies of this latter class include GV39 and GV97.

In a related embodiment, the antibody or antibody fragment is selected from the group consisting of trastuzumab, alemtuzumab (B cell chronic lymphocytic leukemia), gemtuzumab ozogamicin (CD33+ acute myeloid leukemia), hP67.6 (CD33+ acute myeloid leukemia), infliximab (inflammatory bowel disease and rheumatoid arthritis), etanercept (rheumatoid arthritis), rituximab, tositumomab, MDX-210, oregovomab, anti-EGF receptor mAb, MDX-447, anti-tissue factor protein (TF), (Sunol); ior-c5, c5, edrecolomab, ibritumomab tiuxetan, anti-idiotypic mAb mimic of ganglioside GD3 epitope, anti-HLA-Dr10 mAb, anti-CD33 humanized mAb, anti-CD52 humAb, anti-CD1 mAb (ior t6), MDX-22, celogovab, anti-17-IA mAb, bevacizumab, daclizumab, anti-TAG-72 (MDX-220), anti-idiotypic mAb mimic of high molecular weight proteoglycan (I-Mel-1), anti-idiotypic mAb mimic of high molecular weight proteoglycan (I-Mel-2), anti-CEA Ab, hmAbH11, anti-DNA or DNA-associated proteins (histones) mAb, Gliomab-H mAb, GNI-250 mAb, anti-CD22, CMA 676), anti-idiotypic human mAb to GD2 ganglioside, ior egf/r3, anti-ior c2 glycoprotein mAb, ior c5, anti-FLK-2/FLT-3 mAb, anti-GD-2 bispecific mAb, antinuclear autoantibodies, anti-HLA-DR Ab, anti-CEA mAb, palivizumab, bevacizumab, alemtuzumab, BLyS-mAb, anti-VEGF2, anti-Trail receptor; B3 mAb, mAb BR96, breast cancer; and Abx-Cbl mAb.

In one important embodiment, the antibody or antibody fragment is an anti-HER2 antibody, and preferably it is trastuzumab. In another important embodiment, the antibody or antibody fragment is an anti-CD20 antibody, and preferably it is rituximab.

The antibody or antibody fragment may conjugated (covalently or otherwise) to a toxin derived from plant, fungus, or bacteria. The toxin may be selected from the group consisting of A chain toxin, deglycosylated A chain toxin, ribosome inactivating protein, α-sarcin, aspergillin, restrictocin, ribonuclease, diptheria toxin and Pseudomonas exotoxin, but is not so limited. The antibody or antibody fragment may also conjugated to a chemotherapeutic agent, a radioisotope or a cytotoxin. The chemotherapeutic agent may be selected from the group consisting of an anti-metabolite, an anthracycline, a vinca alkaloid, an antibiotic, an alkylating agent, and an epipodophyllotoxin, but is not so limited.

In one embodiment, the antibody or antibody fragment is administered in a sub-therapeutic dose. In one embodiment, the agent of Formula I is administered in a route of administration different from that of the antibody or antibody fragment.

In still other embodiments, the subject is otherwise free of symptoms calling for hematopoietic stimulation. The subject may be non-immunocompromised, but is not so limited. In some embodiments, the subject is genetically immunocompromised, and may be so as a result of a genetic mutation such as in agammaglobulenemia or SCID. In another embodiment, the subject may have an immune deficiency selected from the group consisting of Bruton's agammaglobulinemia, congenital hypogammaglobulinemia, common variable immunodeficiency, and selective immunoglobulin A deficiency. In another embodiment, the subject is elderly (e.g., at least 50 years old). In still another embodiment, the subject is non-immunocompromised as it has not undergone any immunosuppressive therapies such as chemotherapy or radiation.

In one embodiment, the agent of Formula I is administered in an enterically coated form and the antibody or antibody fragment is administered by injection. In another embodiment, the agent of Formula I is administered prior to the antibody or antibody fragment.

In certain embodiments shared by various aspects of the invention, the agent of Formula I is administered in an amount that increases lymphoid tissue (e.g., spleen) levels of IL-1, G-CSF or IL-8 (KC in mouse). In the various embodiments described herein, it is to be understood that the invention embraces induction of either or both IL-1α and IL-1β, and thus a general recitation of IL-1 means both a and β forms. In another embodiment, the agent of Formula I is administered in an amount that does not increase serum IL-1 levels.

In one embodiment, the agent of Formula I is administered 30 minutes to 8 hours prior to the antibody or antibody fragment. In another embodiment, the agent of Formula I is administered 1 to 7 days prior to the antibody or antibody fragment. In yet another embodiment, the agent of Formula I is administered substantially simultaneously with the antibody or antibody fragment. As used herein, the term “substantially simultaneously” means that the compounds are administered within minutes of each other (e.g., within 10 minutes of each other) and intends to embrace joint administration as well as consecutive administration, but if the administration is consecutive it is separated in time for only a short period (e.g., the time it would take a medical practitioner to administer two compounds separately). As used herein, concurrent administration and substantially simultaneous administration are used interchangeably.

In one embodiment, the agent of Formula I is administered after the antibody or antibody fragment. The antibody or antibody fragment may be administered on a first day of multi-day cycle, with the agent of Formula I administered on the remaining days of the cycle. The cycle may be a 2, 3, 4, 5, 6, 7, or more day cycle. The agent of Formula I may be administered once, twice, or more times per day. In one embodiment, the antibody or antibody fragment is administered on the first day of a seven day cycle, followed by a twice daily administration of the agent of Formula I on each of the remaining days of the seven day cycle. The multi-day cycle may be repeated twice, thrice, four times, or more. It may also be repeated for various lengths of time, including but not limited to a week, a month, two months, or more.

The invention provides in yet another aspect a composition comprising an effective amount of an agent of Formula I and an antibody or antibody fragment. In one embodiment, the composition further comprises a pharmaceutically acceptable carrier. In one embodiment, the effective amount is an amount to stimulate antibody dependent cell-mediated cytoxicity. In another embodiment, the effective amount is an amount to treat or prevent cancer. In still another embodiment, the effective amount is an amount to treat or prevent an infectious disease. In one embodiment, the antibody or antibody fragment is an antibody, and it can be selected from the group listed above.

In another aspect, the invention provides a method for stimulating an immune response in a subject comprising administering to a subject in need of immune stimulation an agent of Formula I, and an antigen, in an amount effective to stimulate an antigen-specific immune response, wherein the agent of Formula I is administered at a concentration of greater than 10⁻⁸M. In another embodiment, the agent of Formula I is administered in a route of administration different from that of the antigen.

In one embodiment, the antigen is a cancer antigen. The cancer antigen may be selected from the group consisting of those recited above and MART-1/Melan-A, gp100, adenosine deaminase-binding protein (ADAbp), FAP, cyclophilin b, colorectal associated antigen (CRC)—C017-1A/GA733, carcinoembryonic antigen (CEA), CAP-1, CAP-2, etv6, AML1, prostate specific antigen (PSA), PSA-1, PSA-2, PSA-3, prostate-specific membrane antigen (PSMA), T-cell receptor/CD3-zeta chain, and CD20. The cancer antigen may also be selected from the group consisting of MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-C5). In still another embodiment, the cancer antigen is selected from the group consisting of GAGE-1, GAGE-2, GAGE-3, GAGE-4, GAGE-5, GAGE-6, GAGE-7, GAGE-8, GAGE-9. And in yet a further embodiment, the cancer antigen is selected from the group consisting of BAGE, RAGE, LAGE-1, NAG, GnT-V, MUM-1, CDK4, tyrosinase, p53, MUC family, HER2/neu, p21ras, RCAS1, α-fetoprotein, E-cadherin, α-catenin, β-catenin, γ-catenin, p120ctn, gp100^{Pmel117}, PRAME, NY-ESO-1, cdc27, adenomatous polyposis coli protein (APC), fodrin, Connexin 37, Ig-idiotype, p15, gp75, GM2 ganglioside, GD2 ganglioside, human papilloma virus proteins, Smad family of tumor antigens, Imp-1, P1A, EBV-encoded nuclear antigen (EBNA)-1, brain glycogen phosphorylase, SSX-1, SSX-2 (HOM-MEL-40), SSX-1, SSX-4, SSX-5, SCP-1 and CT-7, and c-erbB-2.

The cancer antigen also includes any of the cancer antigens mentioned infra with respect to other aspects of the invention, such as for example those listed in Table 1.

In one embodiment, the agent of Formula I is administered to the subject prior to the antigen (or the antibody). In another embodiment, the agent of Formula I is administered to the subject 30 minutes to 8 hours before administration of the antigen (or the antibody). In another embodiment, the agent of Formula I is administered to the subject 1 to 7 days before administration of the antigen (or the antibody). In another embodiment, the agent of Formula I is administered to the subject after the antigen (or the antibody). In another embodiment, the agent of Formula I is administered to the subject 30 minutes to 8 hours after administration of the antigen (or the antibody). In another embodiment, the agent of Formula I is administered to the subject 1 to 7 days after administration of the antigen (or the antibody).

In certain embodiments of this and other aspects of the invention, the method further comprises administering an adjuvant to the subject. In one embodiment, the adjuvant is selected from the group consisting of alum, cholera toxin, CpG immunostimulatory nucleic acids, MPL, MPD, and QS-21.

In certain embodiments of the foregoing aspects of the invention, the methods may further comprise treating the subject with a therapy selected from the group consisting of surgery, radiation and chemotherapy. In one embodiment, the agent of Formula I and the antigen (or the antibody) are administered prior to treating the subject with a therapy selected from the group consisting of surgery, radiation and chemotherapy. In another embodiment, the agent of Formula I and the antigen (or antibody) are administered after treating the subject with a therapy selected from the group consisting of surgery, radiation and chemotherapy. In yet another embodiment, the agent of Formula I and the antigen (or antibody) are administered before and after treating the subject with a therapy selected from the group consisting of surgery, radiation and chemotherapy.

In one embodiment, the antigen is a microbial antigen. As used herein, a microbial antigen is an antigen derived from an infectious pathogen, and may include the entire pathogen. The antigen may be peptide, lipid, or carbohydrate in nature, but it is not so limited. The microbial antigen may be selected from the group consisting of a bacterial antigen, a mycobacterial antigen, a viral antigen, a fungal antigen, and a parasitic antigen. The invention intends to embrace various antigens from the infectious pathogens recited herein.

In one embodiment, the bacterial antigen is derived from a bacterial species selected from the group consisting of E. coli, Staphylococcal, Streptococcal, Pseudomonas, Clostridium difficile, LegionelIa, Pneumococcus, Haemophilus, KlebsielIa, Enterobacter, Citrobacter, Neisseria, Shigella, Salmonella, Listeria, Pasteurella, Streptobacillus, Spirillum, Treponema, Actinomyces, Borrelia, Corynebacterium, Nocardia, Gardnerella, Campylobacter, Spirochaeta, Proteus, Bacteriodes, H. pylori, and anthrax.

The mycobacterial antigen may be derived from a mycobacterial species such as M. tuberculosis and M. leprae, but is not so limited.

In another embodiment, the viral antigen is derived from a viral species selected from the group consisting of HIV, Herpes simplex virus 1, Herpes simplex virus 2, cytomegalovirus, hepatitis A virus, hepatitis B virus, hepatitis C virus, human papilloma virus, Epstein Barr virus, rotavirus, adenovirus, influenza A virus, respiratory syncytial virus, varicella-zoster virus, small pox, monkey pox and SARS.

In yet another embodiment, the fungal antigen is derived from a fungal species that causes an infection selected from the group consisting of candidiasis, ringworm, histoplasmosis, blastomycosis, paracoccidioidomycosis, crytococcosis, aspergillosis, chromomycosis, mycetoma infections, pseudallescheriasis, and tinea versicolor infection.

In still another embodiment, the parasitic antigen is derived from a parasite species selected from the group consisting of amebiasis, Trypanosoma cruzi, Fascioliasis, Leishmaniasis, Plasmodium, Onchocerciasis, Paragonimiasis, Trypanosoma brucei, Pneumocystis, Trichomonas vaginalis, Taenia, Hymenolepsis, Echinococcus, Schistosomiasis, neurocysticercosis, Necator americanus, and Trichuris trichuria.

In another aspect, the invention provides a composition comprising an effective amount of an agent of Formula I and an antigen such as a cancer antigen or a microbial antigen. The effective amount may be that amount to treat or prevent cancer, or that amount to treat or prevent an infectious disease. In this and other aspects of the invention, the antigen may be a peptide antigen, or a lipid antigen, but it is not so limited. The antigen can be selected from the groups recited above. In one embodiment, the agent of Formula I is formulated for administration at a dose of greater than 10⁻⁸M.

In yet another aspect, the invention provides a method of preventing an infectious disease in a subject at risk of developing an infectious disease comprising identifying a subject at risk of developing an infectious disease, and administering an agent of Formula I to the subject in an amount effective to induce IL-1.

In one embodiment, the method further comprises administering to the subject a microbial antigen, selected from the groups recited above. In one embodiment, the infectious disease is selected from the group consisting of a bacterial infection, a viral infection, a fungal infection and a parasitic infection, and these can be selected from the groups listed above.

In certain embodiments of this and other aspects of the invention, the subject is HIV negative.

In yet another aspect, the invention provides a composition comprising an effective amount of an agent of Formula I and a microbial antigen, wherein the agent of Formula I is formulated for administration at a dose of greater than 10⁻⁸M. In one embodiment, the effective amount is an amount to treat or prevent an infectious disease. The microbial antigen can be selected from the groups recited above. However, in some embodiments, the microbial antigen is not an HIV antigen.

In yet another aspect, the invention provides a method for stimulating an immune response in a subject having or at risk of having cancer comprising administering to a subject in need of immune stimulation an agent of Formula I, and an antigen, in an amount effective to stimulate an antigen-specific immune response. In one embodiment, the subject is HIV negative. In another embodiment, the subject is a subject having cancer. In yet another embodiment, the subject has or is at risk of developing an infectious disease, and these infectious diseases can be selected from the groups recited above. In one embodiment, the subject is further administered an antigen such as a cancer antigen or a microbial antigen, and either can be selected from the groups recited above. In one embodiment, the method further comprises treating the subject with one or more therapies selected from the group consisting of surgery, radiation and chemotherapy. The timing of administration may vary as described herein. Thus, the agent of Formula I and the antigen may be administered prior to, at the same time as, and/or following surgery, radiation and chemotherapy. In another embodiment, the agent of Formula I and the antigen are administered before and after treating the subject with surgery, radiation and chemotherapy. In yet another embodiment, the subject has not undergone an anti-cancer therapy selected from the group consisting of surgery, radiation and chemotherapy.

In certain embodiments of this and other aspects of the invention, the agent of Formula I is administered to the subject prior to the antigen. In a related embodiment, the agent of Formula I is administered to the subject 30 minutes to 8 hours before administration of the antigen. In still another embodiment, the agent of Formula I is administered to the subject 1 to 7 days before administration of the antigen. The agent of Formula I may be administered in a dose greater than 10⁻⁸ M

The invention provides in still another aspect, a method for stimulating an immune response in a non-immunocompromised subject comprising administering to a subject in need thereof an agent of Formula I, in an amount effective to induce IL-1. In one embodiment, the method can further comprise administering an antigen or an antibody or fragment thereof to the subject. The antigen can be a cancer antigen or a microbial antigen, as taught herein, but it is not so limited. In one embodiment, the subject will have a surgery. In another embodiment, the subject has a skin abrasion from a trauma. In yet another embodiment, the subject is traveling to a region in which a microbial infection is common. In one embodiment, the subject is elderly. In one embodiment, the agent of Formula I and the antigen are formulated together. In another embodiment, the antigen is administered mucosally. In one embodiment, the agent of Formula I is administered orally in an enterically coated form.

In still another aspect of the invention, a method is provided for stimulating an immune response in a genetically immunocompromised subject comprising administering to a subject in need thereof an agent of Formula I, in an amount effective to induce IL-1. In one embodiment, the subject has a genetic deficiency selected from the group consisting of SCID, agammaglobulinemia such as Bruton's agammaglobulinemia and congenital hypogammaglobulinemia, common variable immunodeficiency (CDG), and selective immunoglobulin A deficiency.

In yet a further aspect of the invention, a method is provided for treating a subject having or at risk of developing an interferon (IFN)-responsive condition. The method comprises administering to a subject in need of such treatment an agent of Formula I in an amount effective to induce a therapeutically or prophylactically effective amount of IL-1 in the subject. The method may further comprise identification of a subject having or at risk of developing an IFN-responsive condition. The IFN may be IFNα, IFNα-2b, IFNβ or IFNγ, but is not so limited. In one embodiment, the condition is an IFNγ-responsive condition, and may be selected from the group consisting of viral infections and associated diseases, and cancer. In one embodiment, the subject is HIV positive. In one embodiment, the IFN-responsive condition is a chronic infection selected from the group consisting of a chronic hepatitis B infection, chronic hepatitis C infection, chronic Epstein Barr Virus infection, and tuberculosis. Other disorders include hepatocellular carcinoma, Kaposi's Sarcoma (AIDS-related), thick primary melanomas, and regional lymph node metastases. In one embodiment, the disorder is refractive (i.e., resistant) to prior therapy (e.g., drug treatment) Thus, in one embodiment, the disorder is drug resistant. In another embodiment, the disorder is multiple sclerosis. IFN-responsive conditions are not intended to be so restricted however.

In another embodiment, the method further comprises administering to the subject a second active agent selected from the group consisting of IFNα, pegylated IFN, IFNα-2b, acyclovir, lobucavir, ganciclovir, L-deoxythymidine, clevudine, a therapeutic vaccine, phosphonoformate (PFA), ribavirin (RBV), thymosin alpha-1, 2 3-dideoxy-3-fluoroguanosine (FLG), famciclovir, lamivudine, adefovir dipivoxil, entecavir, emtricitabine, and hepatitis B-specific immunoglobulin.

In certain embodiments of this and other aspects of the invention, the IL-1 is IL-1α or IL-1β.

In a further aspect, the invention provides a method for treating a subject having or at risk of developing cancer comprising administering to a subject in need of such treatment an enzyme inhibitor selected from the group consisting of a tyrosine kinase inhibitor, a CDK inhibitor, a MAP kinase inhibitor, and an EGFR inhibitor, and an agent of Formula I in an amount effective to inhibit the cancer. In one embodiment, the tyrosine kinase inhibitor is selected from the group consisting of Genistein (4′,5,7-trihydroxyisoflavone), Tyrphostin 25 (3,4,5-trihydroxyphenyl), methylene]-propanedinitrile, Herbimycin A, Daidzein (4′,7-dihydroxyisoflavone), AG-126, trans-1-(3′-carboxy-4′-hydroxyphenyl)-2-(2″,5″-dihydroxy-phenyl)ethane, and HDBA (2-Hydroxy5-(2,5-Dihydroxybenzylamino)-2-hydroxybenzoic acid. In another embodiment, the CDK inhibitor is selected from the group consisting of p21, p27, p57, p15, p16, p18, and p19. In another embodiment, the MAP kinase inhibitor is selected from the group consisting of KY12420 (C₂₃H₂₄O₈), CNI-1493, PD98059, 4-(4-Fluorophenyl)-2-(4-methylsulfinyl phenyl)-5-(4-pyridyl) 1H-imidazole. In still a further embodiment, the EGFR inhibitor is selected from the group consisting of Tarceva™ (OSI-774), Iressa (ZD1839), WH1-P97 (quinazoline derivative), LFM-A12 (leflunomide metabolite analog), AG1458. In various embodiments, the amount effective is a synergistic amount.

In yet one more aspect of the invention, a method is provided for treating a subject having or at risk of developing cardiovascular disease comprising administering to a subject in need of such treatment an agent of Formula I in an amount effective to induce an effective amount of IL-1. The method may further comprise identifying a subject in need of such treatment.

In another aspect, the invention provides a method for preventing drug resistance in a subject. The method involves administering to a subject receiving an anti-microbial agent, an agent of Formula I in an amount effective to reduce the risk of resistance to the anti-microbial agent. In one embodiment, the subject is one having or is at risk of developing an infectious disease. Thus, in one embodiment, the infectious disease is selected from the group consisting of a bacterial infection, a viral infection, a fungal infection and a parasitic infection. In one embodiment, the bacterial infection is a Pseudomonas infection. Other drug resistant microbes and the drugs to which they are resistant include Staphylococcus aureus (penicillin), Streptococcus pneumoniae (penicillin), gonorrhea (penicillin), and Enterococcus faecium (penicillin). In one embodiment, the anti-microbial agent is selected from the group consisting of an anti-bacterial agent, an anti-viral agent, an anti-fungal agent, and an anti-parasitic agent.

In still another aspect, the invention provides a method for shortening a vaccination course. As used herein, “shortening a vaccination course” refers to reducing either the number of vaccine administrations (e.g., by injection) or the time between vaccine administrations. This is accomplished by stimulating a more robust immune response in the subject. The method may involve, in one embodiment, administering to a subject in need of immunization an agent of Formula I in an amount effective to induce an antigen-specific immune response to a vaccine administered in a vaccination course, wherein the vaccination course is shortened by at least one immunization. In other embodiments, the vaccination course is shortened by one immunization, two immunizations, three immunizations, or more. The method may involve, in another embodiment, administering to a subject in need of immunization an agent of Formula I in an amount effective to induce an antigen-specific immune response to a vaccine administered in a vaccination course, wherein the vaccination course is shortened by at least one day. In other embodiments, the vaccination course is shortened by one day, two days, three days, four days, five days, six days, one week, two weeks, three weeks, four weeks, one month, two months or more. In one embodiment, the agent of Formula I is administered substantially simultaneously with the vaccine. Immunizations that can be modified in this way include but are not limited to newborn immunizations for HBV; immunizations at for example two months of age for Polio, DTaP, Hib, HBV, Pneumococcus; immunizations at for example four months of age for Polio, DTaP, Hib, Pneumococcus; immunizations at for example six months of age for Polio, DTaP, Hib, HBV, Pneumococcus; immunizations at for example 12-15 months of age for Hib, Pneumococcus, MMR, Varicella; immunizations at for example 15-18 months of age for DtaP; immunizations at for example 4-6 years of age for Polio, DPT, MMR; immunizations at for example 11-12 years of age for MMR; immunizations at for example 14-16 years of age for tetanus-diphtheria (i.e., Td) (with a repeat as a booster every 10 years). As an example, a recommended vaccination course for tetanus/diphtheria includes a primary immunization series given in adults if not received as a child, followed by routine booster doses of tetanus-diphtheria (Td) every 10 years. The method of the invention will allow for a shortened series of vaccinations at the first time point, and may in some instances obviate the need for booster shoots later on. As another example, hepatitis vaccination commonly requires three administrations spaced at least two weeks, and sometimes one month, apart in order to develop full immunity. Using the methods of the invention, it is possible to either reduce the number of injections from three to two or one, or to reduce the time in between injections from weeks or months to days or weeks. Vaccination courses that can be shortened by the method of the invention include but are not limited to: HBV: Hepatitis B vaccine (3 total doses currently recommended); Polio: Inactivated polio vaccine (4 total doses currently recommended); DTaP: Diphtheria/tetanus/acellular Pertussis (3-in-I vaccine; 5 total doses currently recommended); Hib: Haemophilus influenzae type b conjugate vaccine (4 total doses currently recommended); Pneumococcus (Prevnar): Protects against certain forms of Strep. Pneumoniae (3 total doses recommended); MMR: measles/mumps/rubella (3-in-1 vaccine; 2 total doses recommended); Td: Adult tetanus/diphtheria (2-in-1 vaccine; for use in people over age 7). In another embodiment, the compounds of Formula I can be used together with oral polio vaccine.

The invention provides in yet another aspect a method for stimulating an immune response in a subject having cancer comprising administering to a subject in need of such treatment an agent of Formula I in an amount effective to stimulate an antigen-specific immune response, prior to and following a therapy selected from the group consisting of radiation, surgery and chemotherapy. The foregoing embodiments relating to agent of Formula I are equally applicable to this aspect of the invention. The foregoing embodiments relating to cancer are similarly equally applicable to this aspect of the invention.

In one embodiment, the agent of Formula I is administered to the subject 30 minutes to 8 hours before the therapy and 30 minutes to 8 hours after the therapy. In one embodiment, the agent of Formula I is administered in a dose of greater than 10⁻⁸M.

In still another aspect, a method is provided for stimulating an immune response in a subject at risk of developing cancer comprising administering to a subject in need of such treatment an agent of Formula I in an amount effective to stimulate an antigen-specific immune response. In one embodiment, the method further comprises identifying a subject in need of such treatment. In another embodiment, the subject at risk of developing cancer has a familial predisposition to developing cancer. In one embodiment, the familial predisposition is familial colon polyposis. In a related embodiment, the subject has precancerous polyps. In another embodiment, the subject has precancerous HPV lesions. In other embodiments the familial predisposition can include BRCA1- and BRCA2-associated breast cancer, Wilms tumour, colorectal cancer, Li-Fraumeni Syndrome, ovarian cancer, and prostate cancer. In another embodiment, the subject is at risk of developing a cancer that is a metastasis.

The invention in yet another aspect provides a pharmaceutical preparation comprising an agent of Formula I in a dosage of about 0.005 mg/kg to less than 1.0 mg/kg per day, and a pharmaceutically acceptable carrier, wherein the preparation is formulated for injection or in an enterically coated form. In one embodiment, the preparation is provided in a vial or ampoule with a septum.

The invention provides in another aspect a pharmaceutical preparation comprising an agent of Formula I in a dosage of less than 1.0 mg/kg per day, wherein the preparation is provided in a vial or ampoule with a septum. In one embodiment, the dosage is about 0.005 to less than or equal to 0.1 mg/kg per day.

Various embodiments apply equally to the pharmaceutical preparations and compositions provided herein and these embodiments are recited below.

In one embodiment, the preparation is sterile and pyrogen-free. The preparation may further comprise a pharmaceutically acceptable carrier. The pharmaceutically acceptable carrier may comprise a solubilizer, an anti-bacterial preservative, an anti-oxidant or a pharmaceutical adjunct. The anti-oxidant may be sodium bisulfite, but it is not so limited. In another embodiment, the preparation comprises distilled water or reverse-osmosis water. In another embodiment, the anti-bacterial preservative is phenylmercuric nitrate, thimerosal, benzetheonium chloride, benzalkonium chloride, phenol, cresol or chlorobutanol. These embodiments are equally applicable to other aspects of the invention.

The pharmaceutically acceptable carrier may have a pH of less than 5, or a pH of between 2.0 and 5.0, or between 3.0 and 5.0, or between 3.0 and 4.5, or between 3.0 and 4.25, or between 3.0 and 4.0, or between 3.0 and 3.5.

The invention also provides a method of producing the foregoing pharmaceutical preparations, comprising combining the agent of Formula I with a pharmaceutically acceptable carrier. In important embodiments, combining occurs within 2 hours, 1 hour or 30 minutes of administration to a subject.

The compositions of the invention may be provided in a housing such as a container, a box, or a bag. The housing may also contain instructions for use of the composition either thereon or therein. The instructions for use indicate how the contents of the housing are to be used, including timing and dose of administrations. In these latter embodiments, the compositions may be contained in a kit.

In another aspect, the invention provides a kit comprising a housing that comprises an agent of Formula I in a first container, and a pharmaceutically acceptable carrier in a second container, wherein the agent of Formula I is present in a dried form.

In one embodiment, the agent and carrier are sterile and pyrogen-free. In some embodiments, the kit comprises a plurality of first and second containers corresponding to a number of administrations to a subject. In a related embodiment, the first container is a vial or ampoule with a septum. In other embodiments, the second container is a vial or ampoule with a septum.

In yet another aspect, the invention provides a kit comprising a housing that comprises an agent of Formula I dissolved in an acid solution in a first container, and a neutral or basic isotonic diluent in a second container. In one embodiment, the kit further comprises instructions for administering the agent to a subject in need thereof. In another embodiment, the agent, solution and diluent are sterile and pyrogen-free.

In certain embodiments, the acid solution has a pH of less than 5, or a pH between 2.0 and 5.0, or between 3.0 and 5.0, or between 3.0 and 4.5, or between 3.0 and 4.25, or between 3.0 and 4.0, or between 3.0 and 3.5.

In other embodiments, the diluent has a pH greater than 5, or a pH between 5.0 and 8.0, or between 5.0 and 7.5, or between 5.0 and 7.0, or between 5.0 and 6.5, or between 5.0 and 6.0, or between 5.0 and 5.5.

In yet a further aspect, the invention provides a kit comprising an agent of Formula I in a first container, and instructions for diluting the agent in a neutral or acidic injectable diluent. The kit may further comprise a housing comprising the first container and the instructions.

The diluent and acid solution possess similar characteristics as the pharmaceutically acceptable carrier, as described above. In various embodiments, the diluent has a pH of less than 7, or a pH of between 2.0 and 7.0, or between 3.0 and 6.0, or between 3.0 and 5.0, or between 3.0 and 4.25, or between 3.0 and 4.0, or between 3.0 and 3.5.

The kit may further comprise a plurality of first containers corresponding to a number of administrations to a subject. In some embodiments, the first container is a vial or ampoule with a septum.

In all of the foregoing aspects and embodiments of the invention, the agent of Formula I is administered by injection or an enterically coated form. The following embodiments apply equally to the various aspects recited herein. In one embodiment, the agent is formulated in a dosage of about 0.005 mg/kg to less than 1.0 mg/kg per day. In another embodiment, the agent is formulated in a dosage of about 0.005 mg/kg to less than or equal to 0.1 mg/kg per day. In one embodiment, the agent is an agent of Formula II. In another embodiment, the agent is an agent of Formula III. In yet other embodiments, the agent is Ile-boroPro, or Ile-L-boroPro, or L-Ile-L-boroPro. In other embodiments, the agent and/or the pharmaceutically acceptable carrier is sterile and pyrogen-free.

The aspects provided herein share a number of common embodiments. Accordingly, these embodiments will be recited once but it is to be understood that they apply equally to various related aspects of the invention.

These and other aspects of the invention will be described in greater detail below. Throughout this disclosure, all technical and scientific terms have the same meaning as commonly understood by one of ordinary skill in the art to which this invention pertains unless defined otherwise.

These and other aspects of the invention will be described in greater detail below. Throughout this disclosure, all technical and scientific terms have the same meaning as commonly understood by one of ordinary skill in the art to which this invention pertains unless defined otherwise.

### BRIEF DESCRIPTION OF THE FIGURES

FIG. 1. Comparison of the ability of Ile-boroPro to stimulate chemokine production in vivo when administered orally or by subcutaneous injection. Ile-boroPro was administered to mice by oral gavages (open symbols) or by subcutaneous injection (closed symbols) at the doses indicated on the abscissa. Two hours after administration, serum samples were obtained and assayed for DPP-IV activity (Δ/▴), using the fluorogenic substrate Ala-Pro-7-amino-4-trifluoromethyl coumarin, and the chemokine KC (◯/●) by ELISA.

FIG. 2. Comparison of the anti-tumor activity of Ile-boroPro administered orally or by subcutaneous injection in the WEHI 164 mouse tumor model. Mice were inoculated subcutaneously with 4×10⁶ tumor cells and administered Ile-boroPro twice daily from day 2 to day 19 after tumor inoculation, either by oral gavage (open bars) or subcutaneous injection (solid bars). Control mice received saline (hatched bar). The data represent mean tumor volumes±SEM (n=10) were recorded on day 20. Treatment with all 3 doses of Ile-boroPro resulted in significant reduction in tumor sizes (oral administration: P<0.05; subcutaneous administration: P<0.00005).

It is to be understood that the figures are not required for enablement of the invention.

### DETAILED DESCRIPTION OF THE INVENTION

The invention is based in part on the surprising discovery that Ile-boroPro and derivatives thereof are more effective when administered by injection (e.g., subcutaneously, intravenously, intramuscularly, intra-tumor injection, intraperitoneally, and the like) or in an enterically coated form such as an enterically coated pill or capsule. Although not intending to be bound by any particular mechanism, it is believed that when administered neat orally Ile-boroPro and its Ile containing derivatives are susceptible to digestion by an aminopeptidase present in the gastrointestinal tract (particularly the upper GI tract). Administration routes that avoid degradation by aminopeptidases of the upper GI tract (including the stomach) increase the therapeutic efficacy of Formula I agents including importantly Ile-boroPro. As described in greater detail herein, enteric coating of orally administered formulations may be designed to withstand gastric acids and to bypass the stomach prior to dissolution.

The agents of the invention can be used alone or in combination with other therapeutic agents such as antibodies, antigens, etc.

The agents of the invention share the common structure of Formula I:

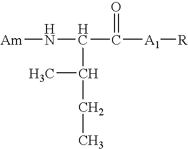

wherein Am and A₁ are L- or D-amino acids, m is an integer between 0 and 10, inclusive; A may be an L- or D-amino acid residue (except that for glycine there is no such distinction) such that each A in Aₘ may be an amino acid residue different from another or all other A in Aₘ; A₁ is bonded to the R with a C bond that is in the L-configuration. By “A₁ is bonded to the R with a C bond that is in the L-configuration” is meant that the absolute configuration of A₁ is like that of an L-amino acid. The R group can be organo boronates, organo phosphonates, fluoroalkylketones, alphaketos, N-peptiolyl-O-(acylhydroxylamines), azapeptides, azetidines, fluoroolefins dipeptide isoesteres, peptidyl (alpha-aminoalkyl) phosphonate esters, aminoacyl pyrrolidine-2-nitriles and 4-cyanothiazolidides, provided that it is capable of reacting with a functional group in the reactive site of FAP-α or other post proline-cleaving enzyme. Post proline-cleaving enzymes are enzymes which have a specificity for removing Xaa-Pro or Xaa-Ala dipeptides (where Xaa represents any amino acid) from the amino terminus of polypeptides. Examples of post-proline cleaving enzymes include, but are not limited to, CD26 and dipeptidyl peptidase IV (DP IV).

In certain embodiments, the agent may be 30, 20, 10 or less than 10 residues in length.

In one embodiment, the agent is also an agent of Formula II:

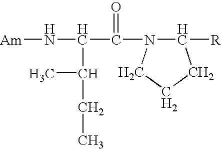

wherein Am is an L- or D-amino acid, m is an integer between 0 and 10, inclusive; A may be an L- or D-amino acid residue (except that for glycine there is no such distinction) such that each A in Aₘ may be an amino acid residue different from another or all other A in Aₘ; the C bonded to R is in the L-configuration. By “the C bonded to R is in the L-configuration” is meant that the absolute configuration of the C is like that of an L-amino acid. The R group can be organo boronates, organo phosphonates, fluoroalkylketones, alphaketos, N-peptiolyl-O-(acylhydroxylamines), azapeptides, azetidines, fluoroolefins dipeptide isoesteres, peptidyl (alpha-aminoalkyl) phosphonate esters, aminoacyl pyrrolidine-2-nitriles and 4-cyanothiazolidides, provided that it is capable of reacting with a functional group in the reactive site of FAP-α or other post proline-cleaving enzyme.

And in yet another embodiment, the agent is also an agent of Formula III:

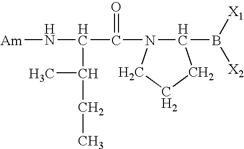

wherein Am is an L- or D-amino acid, m is an integer between 0 and 10, inclusive; A may be an L- or D-amino acid residue (except that for glycine there is no such distinction) such that each A in Aₘ may be an amino acid residue different from another or all other A in Aₘ; the C bonded to B is in the L-configuration; and each X₁ and X₂ is, independently, a hydroxyl group or a group capable of being hydrolyzed to a hydroxyl group in aqueous solution at physiological pH. By “the C bonded to B is in the L-configuration” is meant that the absolute configuration of the C is like that of an L-amino acid.

Thus, the

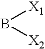

group has the same relationship to the C as the —COOH group of an L-amino acid has to its a carbon. In various embodiments, m is 0; X₁ and X₂ are hydroxyl groups; the inhibitor is Ile-boroPro. In some embodiments, the inhibitor is Ile-L-boroPro. In still other embodiments, inhibitor is L-Ile-L-boroPro.

The agents of the invention may also comprise synthetic moieties derived from synthetic sources such as phage display libraries and chemical combinatorial libraries, provided that the agents maintain the Ile-boroPro element. These additional moieties can be synthesized from peptides or other biomolecules including but not limited to saccharides, fatty acids, sterols, isoprenoids, purines, pyrimidines, derivatives or structural analogs of the above, or combinations thereof and the like. They may be further synthesized from peptoids, random bio-oligomers (U.S. Pat. No. 5,650,489), benzodiazepines, diversomeres such as dydantoins, benzodiazepines and dipeptides, nonpeptidal peptidomimetics with a beta-D-glucose scaffolding, oligocarbamates or peptidyl phosphonates. Many, if not all, of these compounds can be synthesized using recombinant or chemical library approaches. The additional moieties can also be derived from natural sources.

Other useful agents include derivatives of Formulae I, II or III in which each and every A in Aₘ may be independently a non-amino acid residue. Thus, the plurality of A (i.e., Aₘ, wherein m>1) may be a peptide or a peptidomimetic which may include, in whole or in part, non-amino acid residues such as saccharides, fatty acids, sterols, isoprenoids, purines, pyrimidines, derivatives or structural analogs of the above, or combinations thereof and the like. The plurality of A in Aₘ may also be comprised of a combination of amino acid and non-amino acid residues. It also is possible to substitute non-naturally occurring amino acids, such as 2-azetidinecarboxylic acid or pipecolic acid (which have 6-membered, and 4-membered ring structures respectively) for the proline residue.

Representative structures of transition-state analog-based inhibitors of Formula III are referred to as “boroPro” compounds. “BoroPro” refers to the analog of proline in which the carboxylate group (COOH) is replaced with a boronyl group [B(OH)₂]. Alternative compounds of the invention have an analogous structure in which the boronyl group is replaced by, for example, a phosphonate or a fluoroalkylketone, alphaketos, N-peptiolyl-O-(acylhydroxylamines), azapeptides, azetidines, fluoroolefins dipeptide isoesteres, peptidyl (alpha-aminoalkyl) phosphonate esters, aminoacyl pyrrolidine-2-nitriles and 4-cyanothiazolidides. It is to be understood that each and every reactive group described herein can be substituted for the reactive group of Formula III.

These compounds can be provided and used in linear or cyclic form, as described in U.S. Pat. Nos. 4,935,493 and 6,355,614.

All amino acids, with the exception of glycine, contain an asymmetric or chiral carbon and may contain more than one chiral carbon atom. The asymmetric a carbon atom of the amino acid is referred to as a chiral center and can occur in two different isomeric forms. These forms are identical in all chemical and physical properties with one exception, the direction in which they can cause the rotation of plane-polarized light. These amino acids are referred to as being “optically active,” i.e., the amino acids can rotate the plane-polarized light in one direction or the other.

The four different substituent groups attached to the a carbon can occupy two different arrangements in space. These arrangements are not superimposable mirror images of each other and are referred to as optical isomers, enantiomers, or stereo isomers. A solution of one stereo isomer of a given amino acid will rotate plane polarized light to the left and is called the levorotatory isomer [designated (−)]; the other stereo isomer for the amino acid will rotate plane polarized light to the same extent but to the right and is called dextrorotatory isomer [designated (+)].

A more systematic method for classifying and naming stereo isomers is the absolute configuration of the four different substituents in the tetrahedron around the asymmetric carbon atom (e.g., the a carbon atom). To establish this system, a reference compound was selected (glyceraldehyde), which is the smallest sugar to have an asymmetric carbon atom. By convention in the art, the two stereo isomers of glyceraldehyde are designated L and D. Their absolute configurations have been established by x-ray analysis. The designations, L and D, also have been assigned to the amino acids by reference to the absolute configuration of glyceraldehyde. Thus, the stereo isomers of chiral compounds having a configuration related to that of L-glyceraldehyde are designed L, and the stereo isomers having a configuration related to D-glyceraldehyde are designated D, regardless of the direction in which they rotate the plane-polarized light. Thus, the symbols, L and D, refer to the absolute configuration of the four substituents around the chiral carbon.

In general, naturally occurring compounds which contain a chiral center are only in one stereo isomeric form, either D or L. The naturally occurring amino acids are the L stereo isomers; however, the invention also embraces amino acids which can be in the D stereo isomer configuration.

Most amino acids that are found in proteins can be unambiguously named using the D L system. However, compounds which have two or more chiral centers may be in 2″ possible stereo isomer configurations, where n is the number of chiral centers. These stereo isomers sometimes are designated using the RS system to more clearly specify the configurations of amino acids that contain two or more chiral centers. For example, compounds such as threonine isoleucine contain two asymmetric carbon atoms and therefore have four stereo isomer configurations. The isomers of compounds having two chiral centers are known as diastereomers. A complete discussion of the RS system of designating optical isomers for amino acids is provided in Principles in Biochemistry, editor A. L. Lehninger, page 99-100, supra. A brief summary of this system follows.

The RS system was invented to avoid ambiguities when a compound contains two or more chiral centers. In general, the system is designed to rank the four different substituent atoms around an asymmetric carbon atom in order of decreasing atomic number or in order of decreasing valance density when the smallest or lowest-rank group is pointing directly away from the viewer. The different rankings are well known in the art and are described on page 99 of Lehninger (supra). If the decreasing rank order is seen to be clock-wise, the configuration around the chiral center is referred to as R; if the decreasing rank order is counter-clockwise, the configuration is referred to as S. Each chiral center is named accordingly using this system. Applying this system to threonine, one skilled in the art would determine that the designation, L-threonine, refers to (2S, 3R)-threonine in the RS system. The more traditional designations of L-, D-, L-allo, and D-allo, for threonine have been in common use for some time and continue to be used by those of skill in this art. However, the R S system increasingly is used to designate the amino acids, particularly those which contain more than one chiral center.

The agents of the invention may be in some instances substantially optically pure. That is, at least 90%, 92%, 94%, 95%, 96%, 97%, 98% or 99% of the carbon atoms bearing boron are of the L-configuration in some embodiments. Methods for synthesizing optically pure isomers of Formulae I, II, and III agents are disclosed in published PCT application WO 93/08259.

The agents of Formula I can work either alone (i.e., as a monotherapy) or together with another therapeutic agent. The agents are able to inhibit post proline-cleaving enzymes such as those described herein. In particular, the agents inhibit FAPα which is present in reactive stromal fibroblasts of cancers and in some cancers themselves.

The agents can be used together with other therapeutic agents in part based on the discovery that the agents stimulate a variety of cytokines and chemokines that in turn stimulate the immune system. The resultant immune stimulation can thus be exploited to enhance the efficacy of immune based therapies such as passive (i.e., immunoglobulin) immunotherapy or active immunization with antigens.

Thus, in one aspect, the invention provides methods that exploit the synergy that is achieved when the compounds of Formula I are used together with antibodies or fragments thereof. In another aspect, the invention provides methods for stimulating an antigen specific immune response by administering the agents of Formula I together with antigens. The antigens may be targeted to particular cell types or tissues (see, for example, Corixa targeted antigens). The antibodies and antigens that can be used in the methods of the invention are not restricted to those that are cancer specific, and as described in greater detail herein can apply to a broad range of conditions (e.g., infectious diseases).

Thus the invention provides methods and products for the more effective treatment of cancer using agents of Formula I in combination with cancer specific antibodies. In one embodiment, the combination is synergistic, resulting in greater than additive effects than would otherwise be expected using the agents separately. In other embodiments, the combination is additive.

Antibodies specific for tumor or cancer antigens can suppress tumor growth in vivo via a variety of mechanisms. Antibody dependent cell-mediated cytotoxicity, complement mediated cell lysis, targeting of chemically linked toxins, inhibition of tumor cell division, and induction of tumor cell apoptosis have all been described as mechanisms by which immunoglobulins specific for tumor antigens suppress tumor growth in the treatment of cancer. Although antibody-based treatments for cancer can be effective, they do not completely suppress tumor development and progression in all subjects.

The goal of immunotherapy is to augment a patient's immune response to an established tumor. Different types of cells that can kill tumor targets in vitro and in vivo have been identified: natural killer cells (NK cells), cytolytic T lymphocytes (CTLs), lymphokine-activated killer cells (LAKs), activated macrophages, and neutrophils. NK cells can kill tumor cells without having been previously sensitized to specific antigens, and the activity does not require the presence of class I antigens encoded by the major histocompatibility complex (MHC) on target cells. NK cells are thought to participate in the control of nascent tumors and in the control of metastatic growth. In contrast to NK cells, CTLs can kill tumor cells only after they have been sensitized to tumor antigens and when the target antigen is expressed on the tumor cells that also express MHC class I. CTLs are thought to be effector cells in the rejection of transplanted tumors and of tumors caused by DNA viruses. LAK cells are a subset of null lymphocytes distinct from the NK and CTL populations. Activated macrophages and neutrophils can directly kill tumor cells in a manner that is not antigen dependent nor MHC restricted. In addition, neutrophils can inhibit tumor growth by killing endothelial cells of the vasculature that provide blood supply to the tumor. Thus, activated macrophages and neutrophils are thought to decrease the growth rate of the tumors they infiltrate.

Agents of Formula I can suppress a number of different mouse tumors. It has now been demonstrated that these compounds, when administered to tumor-bearing mice, rapidly stimulate the production of growth factors, cytokines and chemokines. These mediators collectively stimulate the proliferation, activation and chemoattraction to the tumor microenvironment of effector cells involved in both non-adaptive (innate) and immune lysis or growth inhibition of tumor cells. The immune and non-immune effector cell populations mobilized and/or activated by agents of Formula I enhance the tumor suppressive effects of anti-cancer antibodies.

Examples of effector cells involved in the anti-tumor effects of Formula I agents are given below. Although not intending to be bound by any particular mechanism, a brief description of how each cell type can cooperate with tumor-specific antibodies in the lysis or growth inhibition of tumor cells is provided herein.

Tumor-infiltrating T cells, including cytotoxic T lymphocytes (CTL), that either lyse or inhibit tumor growth will suppress tumors by a mechanism of antigen-recognition that is different from that of antibodies. Thus, tumor-specific T cells can augment tumor cell lysis or growth inhibition initiated by antibody-based therapeutics.

Macrophage/monocyte, neutrophil, eosinophil, natural killer cells, and lymphokine activated killer cells are also activated by Formula I agents. Individually or collectively, these effector cell types can either lyse tumor cells or suppress their growth in ligand-receptor mediated interactions that lack immunological specificity. The activities of these cells can account for the innate or non-adaptive immune responses against tumors stimulated by Formula I agents. In addition, all of these cell types possess receptors that bind to the Fc portion of immunoglobulin and are referred to as Fc receptors. Fc receptors can bind to antibodies that are specifically bound to tumor cells by their antigen-binding regions. Therefore, since each effector cell possesses cytotoxicity or growth inhibitory activity against tumor cells, the antibody-mediated interaction targets this activity specifically against the tumor. The mechanism can therefore increase the efficiency with which these otherwise non-specific effector cells suppress tumor growth. The process is frequently referred to as antibody dependent cell-mediated cytotoxicity (ADCC).

Thus, in one aspect, the invention provides a method for stimulating ADCC in a subject. The method comprises administering an anti-cancer antibody or antibody fragment and an agent of Formula I to a subject having or at risk of developing cancer in an amount effective to stimulate antibody dependent cell-mediated cytotoxicity in the subject. In some embodiments, the amount effective to stimulate antibody dependent cell-mediated cytotoxicity is a synergistic amount.

The agents of Formula I are useful in non-cancer methods as well. For example, they can used in methods for inducing mucosal immunity. The mucosal surface is frequently in contact with infectious pathogens such as bacteria, viruses and fungi, and thus an enhanced immune response at this surface would benefit a subject greatly. The compositions provided herewith could also be used, as described below, for a variety of mucosal malignancies. Mucosal immunity generally involves immunoglobulin of the secretory IgA (s-IgA) isotype, and accordingly, antibodies of this isotype could be used together with the agents of Formula I, although such antibodies are not so limited. The agents of Formula I are useful in stimulating both cell-mediated immune responses and antibody-mediated immune responses at mucosal surfaces. Mucosal surfaces include nasal, oral, rectal, vaginal and gastrointestinal surfaces. In these methods, the agent of Formula I is however generally not administered to the mucosal surface unless it is provided in an enterically coated form. The other therapeutic agents which work in concert with agents of Formula I however can be administered directly to a mucosal surface, although this is not required.

The novel observation that Formula I agents induce the production of IL-1 indicates that they can be used for a number of indications that are mediated fully or in part by IL-1 and downstream IL-1 signaling events. Some of these indications are recited herein as targets of monotherapy or combination therapy using Formula I agents.

Formula I agents can be used either alone or in combination with other active agents to treat viral infections, particularly chronic infections, and more particularly chronic hepatitis C infection. Currently, most but not all hepatitis C subjects are administered IFNα. Subjects that are also HIV positive fair worse with this treatment. It has been found according to the invention that hepatitis C infected subjects, and especially those subjects resistant or non-responsive to IFNα treatment, can be treated using Formula I compounds. In some instances, the Formula I agents can be administered with IFNα (which may be in pegylated form), and optionally with ribavirin also. In these subjects, Formula I agents can also be used together with other small molecule drugs that are currently being tested for hepatitis C infection.

The agents are also suitable for treatment of hepatitis B infection. In this latter indication, Formula I compounds can be used alone or together with IFN as well as various small molecule drugs being developed, such as IFNα-2b, acyclovir, lobucavir, ganciclovir, L-deoxythymidine, clevudine, a therapeutic vaccine, phosphonoformate (PFA), ribavirin (RBV) and thymosin alpha-1; and nucleotide and nucleoside analogues such as 2 3-dideoxy-3-fluoroguanosine (FLG), famciclovir, lamivudine, adefovir dipivoxil, entecavir, and emtricitabine. Formula I agents can also be used with hepatitis B-specific immunoglobulin.

The use of Formula I agents with lamivudine is particularly interesting as lamivudine is reportedly associated with drug resistance. The combined use of Formula I agents with lamivudine can reduce or eliminate the risk of drug resistance. Formula I agents may be used in subjects already treated with lamivudine who have demonstrated drug resistance. These latter aspects of the invention apply equally to other indications for which drug resistance has been observed or is suspected. Other bacteria that have been associated with drug resistance include Staphylococcus aureus (resistance to penicillin), Streptococcus pneumoniae (resistance to penicillin), gonorrhea (resistance to penicillin), and Enterococcus faecium (penicillin). In other instances, it may be desirable to use Formula I agents over standard drug therapy if the drug therapy is not particularly suited to a subject or induces intolerable side effects in a patient specific manner.

Formula I agents can also be used in the treatment of tuberculosis, either alone (i.e., as a substitute for currently available drug treatments such as antibiotic therapy), or in combination with those antibiotics.

The ability of Formula I agents to induce cytokines, and in particular IL-1, also indicates that these agents are useful in vaccine induced immunity, including both humoral and cell-mediated immunity. The ability to enhance cellular mediated immunity is useful, inter alia, in the treatment or prevention of viral infections, and in particular, HIV infection. As described in greater detail below, Formula I agents can be used together with vaccines such as those to small pox virus (e.g., BVL).

Induction of IL-1 indicates that Formula I agents can be used to activate macrophages. This in turn can be exploited to reduce plaque formation in cardiovascular disease. Plaque engulfing macrophages can be activated following Formula I agent administration.

Indications relating to immune deficiency can also be treated using Formula I agents. These indications include congenital deficiencies, some of which are described in greater detail herein. Examples include the syndromes commonly referred to as congenital disorder of glycosylation (CDG). Another congenital indication is the immunoglobulin deficiency common variable immunodeficiency (CVID) which is characterized by low IgG and IgA, and in some instances low IgM. Subjects having CVID can present with other clinical manifestations including gastrointestinal problems, granulomatous inflammation, cutaneous features, unusual presentations of enteroviral and mycoplasma infection, an increased incidence of autoimmunity, and a predisposition to lymphoma and stomach cancer. Other congenital indications include agammaglobulinemias such as Bruton's agammaglobulinemia and congenital hypogammaglobulinemia, selective immunoglobulin A deficiency, and severe combined immunodeficiency (i.e., SCID, a T cell deficiency). Immune deficiencies that include low or no immunoglobulin production can be treated using Formula I agents alone, and in some instances, preferably with the antibodies described herein. Other immune deficiencies include amyotrophic lateral sclerosis (ALS), systemic lupus erythematosus, rheumatoid arthritis, Hashimoto's disease, chronic immune thrombocytopenic purpura (chronic ITP), and the like.

As indicated above, Formula I agents are therapeutically and prophylactically useful for indications which are responsive to IFN therapy. The IFN therapy may be IFNα, IFNβ, or IFNγ therapy, but is not so limited. A further example of this is multiple sclerosis. Others include tuberculosis, chronic Epstein Barr Virus (EBV) infection, and chronic hepatitis (e.g., chronic hepatitis C), viral hepatitis (e.g., hepatitis C), hepatocellular carcinoma, Kaposi's Sarcoma (AIDS-related), thick primary melanomas, and regional lymph node metastases. Examples of conditions responsive to IFNγ therapy include but are not limited to viral infections and associated diseases and cancer.

One advantage of using Formula I agents in place of IFN therapy is that Formula I agents are less expensive and easier to administer than IFN. These and other conditions can be immunosuppressive and therefore Formula I agents can be used to enhance immunity in such subjects. Other chronic immunosuppressive conditions can arise from pharmaceutical use such as the use of deliberate anti-inflammatories such as cox-1 or cox-2 inhibitors celecoxib (Celebrex), rofecoxib (Vioxx), naproxen (Naprosyn), non-steroidal anti-inflammatory drugs (NSAIDS) such as ibuprofen (Motrin, Advil), fenoprofen, indomethacin, and valdecoxib (Bextra), and aspirin; substance abuse such as the alcoholism, intravenous drug use, morphine use; chronic infections or disease states such as gingivitis, osteomyelitis, diabetes types I and II, chronic granulomas, Pneumocystis carinii pneumonia (PCP) infection, recurrent fungal/yeast infections, non-Hodgkin's lymphoma, and Kaposi's Sarcoma.

As a prophylaxis, Formula I agents can be used to enhance immunity in a subject at risk of developing a condition that is immunologically responsive. For example, a subject may be administered a Formula I agent when it is at risk of developing the flu. As another example, a subject having or at risk of having angina may be administered a Formula I agent.

A subject shall mean a human or animal including but not limited to a dog, cat, horse, cow, pig, sheep, goat, chicken, rodent e.g., rats and mice, primate, e.g., monkey, and fish or aquaculture species such as fin fish (e.g., salmon) and shellfish (e.g., shrimp and scallops). Subjects suitable for therapeutic or prophylactic methods include vertebrate and invertebrate species. Subjects can be house pets (e.g., dogs, cats, fish, etc.), agricultural stock animals (e.g., cows, horses, pigs, chickens, etc.), laboratory animals (e.g., mice, rats, rabbits, etc.), zoo animals (e.g., lions, giraffes, etc.), but are not so limited. Although many of the embodiments described herein relate to human disorders, the invention is also useful for treating other nonhuman vertebrates.

In some embodiments, the agents of Formula I are administered by injection to a subject that is intolerant of orally administered therapeutic agents. These subjects may be incapable of tolerating orally administered therapeutic agents primarily due to nausea. The nausea may be caused by the orally administered therapeutic agents, other therapeutic agents administered by non-oral routes, or other treatment modalities, such as for example radiation. Orally administered agents include orally administered boronic acids (e.g., Val-boroPro). Accordingly, these subjects may have been treated with another boronic acid prior to administration with the agents of Formula I, or with one or more other therapeutic agents.

In still other embodiments, the subjects may be genetically immunocompromised, meaning that they harbor a genetic mutation that renders them immunocompromised even in the absence of an infectious or exogenous procedure. Such subjects may have for example a genetic mutation such as in agammaglobulinemia or SCID. These subjects may be treated according to the invention routinely or only when they are at a higher risk of developing an infectious disease e.g., when traveling to a region where infections are common, when having surgery, when having a skin abrasion, etc.

In still other embodiments, the methods taught herein are intended for use in elderly subjects. As used herein, an elderly subject is one that is at least 50 years old, preferably at least 60 years old, more preferably at least 70 years old, and most preferably at least 75 years old.

In some embodiments, the agents of Formula I may be administered to a subject following administration of other boronic acids. The purpose of prior administration of other boronic acids is to saturate sites that would otherwise bind the agents of Formula I without any therapeutic benefit. An example of a boronic acid that may be administered prior to administration of Formula I agents is Pro-boroPro.

The agents can be used alone or in combination to treat disorders characterized by abnormal mammalian cellular proliferation. An abnormal mammalian cell proliferation disorder or condition, as used herein, refers to a localized region of cells which may exhibit an abnormal (e.g., increased) rate of division as compared to their normal tissue counterparts or an abnormal response to growth or inhibitory signals. These conditions include but are not limited to conditions involving solid tumor masses of benign, pre-malignant or malignant character. These conditions also include the cancers recited herein.

The combination therapy is administered to subjects having or at risk of developing cancer. A subject having a cancer is a subject that has detectable cancerous cells. A subject at risk of developing a cancer is one who has a higher than normal probability of developing cancer. These subjects include, for instance, subjects having a genetic abnormality that has been demonstrated to be associated with a higher likelihood of developing a cancer, subjects having a familial disposition to cancer, subjects exposed to cancer causing agents (i.e., carcinogens) such as tobacco, asbestos, or other chemical toxins, and subjects previously treated for cancer and in apparent remission.

“Cancer” as used herein refers to an uncontrolled growth of cells which interferes with the normal functioning of the bodily organs and systems. Hemopoietic cancers, such as leukemia, are able to outcompete the normal hemopoietic compartments in a subject, thereby leading to hemopoietic failure (in the form of anemia, thrombocytopenia and neutropenia) ultimately causing death.

A cancer cell is a cell that divides and reproduces abnormally due to a loss of normal growth control. Cancer cells almost always arise from at least one genetic mutation. In some instances, it is possible to distinguish cancer cells from their normal counterparts based on profiles of expressed genes and proteins, as well as to the level of their expression. Genes commonly affected in cancer cells include oncogenes, such as ras, neu/HER2/erbB, myb, myc and abl, as well as tumor suppressor genes such as p53, Rb, DCC, RET and WT. Cancer-related mutations in some of these genes leads to a decrease in their expression or a complete deletion. In others, mutations cause an increase in expression or the expression of an activated variant of the normal counterpart.

The term “tumor” is usually equated with neoplasm, which literally means “new growth” and is used interchangeably with “cancer.” A “neoplastic disorder” is any disorder associated with cell proliferation, specifically with a neoplasm. A “neoplasm” is an abnormal mass of tissue that persists and proliferates after withdrawal of the carcinogenic factor that initiated its appearance. There are two types of neoplasms, benign and malignant. Nearly all benign tumors are encapsulated and are noninvasive; in contrast, malignant tumors are almost never encapsulated but invade adjacent tissue by infiltrative destructive growth. This infiltrative growth can be followed by tumor cells implanting at sites discontinuous with the original tumor. The method of the invention can be used to treat neoplastic disorders in humans, including but not limited to: sarcoma, carcinoma, fibroma, leukemia, lymphoma, melanoma, myeloma, neuroblastoma, rhabdomyosarcoma, retinoblastoma, and glioma as well as each of the other tumors described herein.

Cancers that migrate from their original location and seed vital organs (thereby giving rise to metastatic lesions) can eventually lead to the death of the subject through the functional deterioration of the affected organs. A metastasis is a region of cancer cells, distinct from the primary tumor location resulting from the dissemination of cancer cells from the primary tumor to other parts of the body. Thus, subjects with metastatic tumors can also be treated according to the invention. In some embodiments, the metastatic tumors are of epithelial origin. Carcinomas may metastasize to bone, as has been observed with breast cancer, and liver, as is sometimes the case with colon cancer. The methods of the invention are intended to treat metastatic tumors regardless of the site of the metastasis and/or the site of the primary tumor. In preferred embodiments, the metastases are of epithelial origin.

Cancers include, but are not limited to, basal cell carcinoma, biliary tract cancer; bladder cancer; bone cancer; brain and CNS cancer; breast cancer; cervical cancer; choriocarcinoma; colon and rectum cancer; connective tissue cancer; cancer of the digestive system; endometrial cancer; esophageal cancer; eye cancer; cancer of the head and neck; gastric cancer; germ cell tumors; intra-epithelial neoplasm; Kaposi's sarcoma; kidney cancer; larynx cancer; leukemia (e.g., acute myeloid leukemia, acute lymphoid leukemia, chronic myeloid leukemia and chronic lymphoid leukemia); liver cancer; lung cancer (e.g. small cell and non-small cell); lymphoma including Hodgkin's and Non-Hodgkin's lymphoma; melanoma; myeloma; neuroblastoma; oral cavity cancer (e.g., lip, tongue, mouth, and pharynx); ovarian cancer; pancreatic cancer; prostate cancer; renal cell cancer; retinoblastoma; rhabdomyosarcoma; rectal cancer; renal cancer; cancer of the respiratory system; sarcoma; skin cancer; stomach cancer; stromal tumors; testicular cancer; thyroid cancer; uterine cancer; cancer of the urinary system, as well as other carcinomas and sarcomas.

Carcinomas are cancers of epithelial origin that include, but are not limited to, acinar carcinoma, acinous carcinoma, alveolar adenocarcinoma (also called adenocystic carcinoma, adenomyoepithelioma, cribriform carcinoma and cylindroma), carcinoma adenomatosum, adenocarcinoma, carcinoma of adrenal cortex, alveolar carcinoma, alveolar cell carcinoma (also called bronchiolar carcinoma, alveolar cell tumor and pulmonary adenomatosis), basal cell carcinoma, carcinoma basocellulare (also called basaloma, or basiloma, and hair matrix carcinoma), basaloid carcinoma, basosquamous cell carcinoma, breast carcinoma, bronchioalveolar carcinoma, bronchiolar carcinoma, bronchogenic carcinoma, cerebriform carcinoma, cholangiocellular carcinoma (also called cholangioma and cholangiocarcinoma), chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epibulbar carcinoma, epidermoid carcinoma, carcinoma epitheliale adenoides, carcinoma exulcere, carcinoma fibrosum, gelatiniform carcinoma, gelatinous carcinoma, giant cell carcinoma, gigantocellulare, glandular carcinoma, granulosa cell carcinoma, hair-matrix carcinoma, hematoid carcinoma, hepatocellular carcinoma (also called hepatoma, malignant hepatoma and hepatocarcinoma), Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, lenticular carcinoma, carcinoma lenticulare, lipomatous carcinoma, lymphoepithelial carcinoma, carcinoma mastitoides, carcinoma medullare, medullary carcinoma, carcinoma melanodes, melanotic carcinoma, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, carcinoma nigrum, oat cell carcinoma, carcinoma ossificans, osteoid carcinoma, ovarian carcinoma, papillary carcinoma, periportal carcinoma, preinvasive carcinoma, prostate carcinoma, renal cell carcinoma of kidney (also called adenocarcinoma of kidney and hypernephoroid carcinoma), reserve cell carcinoma, carcinoma sarcomatodes, scheinderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, carcinoma vilosum. In preferred embodiments, the methods of the invention are used to treat subjects having cancer of the breast, cervix, ovary, prostate, lung, colon and rectum, pancreas, stomach or kidney.

Another particularly important cancer type is sarcomas. Sarcomas are rare mesenchymal neoplasms that arise in bone and soft tissues. Different types of sarcomas are recognized and these include: liposarcomas (including myxoid liposarcomas and pleiomorphic liposarcomas), leiomyosarcomas, rhabdomyosarcomas, malignant peripheral nerve sheath tumors (also called malignant schwannomas, neurofibrosarcomas, or neurogenic sarcomas), Ewing's tumors (including Ewing's sarcoma of bone, extraskeletal (i.e., non-bone) Ewing's sarcoma, and primitive neuroectodermal tumor [PNET]), synovial sarcoma, angiosarcomas, hemangiosarcomas, lymphangiosarcomas, Kaposi's sarcoma, hemangioendothelioma, fibrosarcoma, desmoid tumor (also called aggressive fibromatosis), dermatofibrosarcoma protuberans (DFSP), malignant fibrous histiocytoma (MFH), hemangiopericytoma, malignant mesenchymoma, alveolar soft-part sarcoma, epithelioid sarcoma, clear cell sarcoma, desmoplastic small cell tumor, gastrointestinal stromal tumor (GIST) (also known as GI stromal sarcoma), osteosarcoma (also known as osteogenic sarcoma)-skeletal and extraskeletal, and chondrosarcoma.

The cancers to be treated may be refractory cancers. A refractory cancer as used herein is a cancer that is resistant to the ordinary standard of care prescribed. These cancers may appear initially responsive to a treatment (and then recur), or they may be completely non-responsive to the treatment. The ordinary standard of care will vary depending upon the cancer type, and the degree of progression in the subject. It may be a chemotherapy, surgery, or radiation, or a combination thereof. Those of ordinary skill in the art are aware of such standards of care. Subjects being treated according to the invention for a refractory cancer therefore may have already been exposed to another treatment for their cancer. Alternatively, if the cancer is likely to be refractory (e.g., given an analysis of the cancer cells or history of the subject), then the subject may not have already been exposed to another treatment. Examples of refractory cancers include but are not limited to leukemias, Non-Hodgkin's lymphoma, melanomas, renal cell carcinomas, colon cancer, colon cancer, liver (hepatic) cancers, pancreatic cancer, and lung cancer.

The invention can also be used to treat cancers that are immunogenic. Cancers that are immunogenic are cancers that are known to (or likely to) express immunogens on their surface or upon cell death. These immunogens are in vivo endogenous sources of cancer antigens and their release can be exploited by the methods of the invention in order to treat the cancer. Examples of immunogenic cancers include those listed in Table 1, including malignant melanoma and renal cell cancer.

Since the invention is premised, in part, on the finding that agents of Formula I are able to inhibit FAP-α, a cell surface marker of reactive stromal fibroblasts, in one aspect, the invention involves treating conditions involving a tumor mass which contains or is dependent upon the presence of reactive stromal fibroblasts at some point during its development. As used herein, reactive fibroblasts are fibroblasts which have been activated to express proteins such as receptors and growth factors which, in some instances, have a positive effect and, in other instances, have a negative effect on cellular proliferation and growth of the fibroblasts themselves, and other cell types such as malignant cells of a carcinoma or epithelial metastasis.

The methods of the invention are also directed towards the treatment of subjects with melanoma. Melanomas are tumors arising from the melanocytic system of the skin and other organs. Examples of melanoma include lentigo malignant melanoma, superficial spreading melanoma, nodular melanoma, and acral lentiginous melanoma.

It is to be understood that in other embodiments, the subjects can be treated with Formula I compounds without any other therapy. In some important embodiments of the invention, the methods are particularly directed to subjects at high risk of cancer, such as those predisposed for familial (e.g., familial colon polyposis, BRCAI— or BRCA2-associated breast cancer, Wilms tumour, colorectal cancer, Li-Fraumeni Syndrome, ovarian cancer, and prostate cancer), or non-familial genetic reasons. Subjects at high risk are also those that manifest pre-cancerous symptoms such as pre-cancerous polyps (e.g., in colon cancer), or pre-cancerous lesions (e.g., in HPV-induced cervical cancer).

The compositions and methods of the invention in certain instances may be useful for replacing existing surgical procedures or drug therapies, although in most instances the present invention is useful in improving the efficacy of existing therapies for treating such conditions. Accordingly combination therapy may be used to treat the subjects that are undergoing or that will undergo a treatment for inter alia cancer or infectious disease. For example, the agents may be administered to a subject in combination with another anti-proliferative (e.g., an anti-cancer) therapy. Suitable anti-cancer therapies include surgical procedures to remove the tumor mass, chemotherapy or localized radiation. The other anti-proliferative therapy may be administered before, concurrent with, and/or after treatment with the agent of the invention. There may also be a delay of several hours, days and in some instances weeks between the administration of the different treatments, such that the agent may be administered before or after the other treatment. In some embodiments, the agents of Formula I may be administered with or without the antigens or antibodies, prior to the administration of the other anti-proliferative treatment (e.g., prior to surgery, radiation or chemotherapy), although the timing is not so limited.

Although not intending to be bound by any particular mechanism, it is proposed that the administration of Formula I compounds inducing memory within the immune cell compartment, for example, by the induction of memory T cells, and B cells. This is believed to occur via the cytokine cocktail that is induced by compounds of Formula I, particularly the induction of IL-1. The ability to generate memory T cells can enhance immune responses to, for example, cancerous cells that are remaining following a surgical procedure, or following chemotherapy or radiation.

The invention further contemplates the use of Formula I compounds in cancer subjects prior to and following surgery, radiation or chemotherapy in order to create memory immune cells to the cancer antigen. In this way, memory cells of the immune system can be primed with cancer antigens and thereby provide immune surveillance in the long term. This is particularly suited to radiotherapy of subjects where immune cells so primed can invade a tumor site and effectively clear any remaining tumor debris. This in turn promotes further immunity to the cancer, particularly to antigens that might not have been exposed in the context of a tumor mass pre-treatment.

The other therapeutic agents that can be administered to a subject together with the agents of Formula I are recited herein, and include chemotherapeutic, antibodies and antibody fragments, and antigens.

The agents of Formula I can also be used to prevent or treat infectious diseases such as bacterial, viral, fungal, parasitic and mycobacterial infections. The agents are able to stimulate innate immunity (i.e., immunity mediated by neutrophils, macrophages, NK cells and eosinophils) and/or adaptive immunity (i.e., immunity mediated by T cells and B cells). The growth factors, cytokines and chemokines stimulated by the compounds of Formula I (e.g., Val-boroPro (PT-100)) can stimulate these cells and thereby enhance an immune response to a foreign pathogen. As an example, IL-1 rapidly activates innate immunity. Therefore, Formula I compounds can be used to activate innate immunity via IL-1β induction, and this in turn can provide an initial defense against any infectious agent.

The agents of Formula I can also be used prophylactically to prevent infection during periods of heightened risk, including for example flu season, epidemics, and travel to places where the risk of pathogen exposure is high. Many of the cytokines and chemokines induced by Formula I compounds can prime a subject and prepare it for passive exposure to a pathogen. The rate at which Formula I compounds stimulate these cytokines and chemokines (e.g., IL-1β) is useful particularly where pathogen exposure cannot be anticipated.

Thus, the methods of the invention can be used in the treatment or prevention of infectious diseases such as bacterial infections, mycobacterial infections, viral infections, fungal infections and parasitic infections.

Examples of bacterial infections include E. coli, Streptococcal infections, Staphylococcal infections, Pseudomonas infections, Clostridium difficile, Legionella infections, Pneumococcus infection, Haemophilus infections (e.g., Haemophilus influenzae infections), Klebsiella infections, Enterobacter infections, Citrobacter infections, Neisseria infections (e.g., N. meningitidis infection, N. gonorrhoeae infection), Shigella infections, Salmonella infections, Listeria infections (e.g., L. monocytogenes infection), Pasteurella infection (e.g., Pasteurella multocida infection), Streptobacillus infection, Spirillum infection, Treponema inection (e.g., Treponema pallidum infection), Actinomyces infection (e.g., Actinomyces israelli infection), Borrelia infection, Corynebacterium infection, Nocardia infection, Gardnerella infections (e.g., Gardnerella vaginalis infection), Campylobacter infections (e.g., Campylobacter fetus infection), Spirochaeta infections, Proteus infections, Bacteriodes infections, H. pylori, and anthrax.

Examples of viral infections include HIV infection, Herpes simplex virus 1 and 2 infections (including encephalitis, neonatal and genital forms), human papilloma virus infection, cytomegalovirus infection, Epstein Barr virus infection, Hepatitis virus A, B and C infections, rotavirus infection, adenovirus infection, influenza A virus infection, respiratory syncytial virus infection, varicella-zoster virus infections, small pox infection, monkey pox infection, and SARS infection. In some embodiments, the methods are not intended to treat or prevent HIV infection.

Examples of fungal infections include candidiasis infection, ringworm, histoplasmosis infection, blastomycosis infections, paracoccidioidomycosis infections, crytococcosis infections, aspergillosis infections, chromomycosis infections, mycetoma infections, pseudallescheriasis infection, and tinea versicolor infection.

Examples of parasite infections include both protozoan infections and nematode infections. These include amebiasis, Trypanosoma cruzi infection (i.e., Chagas' disease), Fascioliasis (e.g., Facioloa hepatica infection), Leishmaniasis, Plasmodium infections (e.g., malaria causing Plasmodium species infections, e.g., P. falciparum, P. knowlesi, P. malariae,) Onchocerciasis, Paragonimiasis, Trypanosoma brucei infection (i.e., Sleeping sickness), Pneumocystis infection (e.g., Pneumocystis carinii infection), Trichomonas vaginalis infection, Taenia infections, Hymenolepsis infections (e.g., Hymenolepsis nana infection), Echinococcus infections, Schistosomiasis (e.g., Schistosoma mansoni infection), neurocysticercosis, Necator americanus infection, and Trichuris trichuria infections.

Other infections that can be treated according to the methods of the invention include Chlamydia infection, mycobacterial infection such as tuberculosis and leprosy, and Rickettsiae.

The foregoing lists of infections are not intended to be exhaustive but rather exemplary. Those of ordinary skill in the art will identify other infections that are amenable to prevention and treatment using the methods of the invention.

Subjects having an infectious disease are those that exhibit symptoms of infectious disease (e.g., rapid onset, fever, chills, myalgia, photophobia, pharyngitis, acute lymphadenopathy, splenomegaly, gastrointestinal upset, leukocytosis or leukopenia) and in whom infectious pathogens or byproducts thereof can be detected. Tests for diagnosing infectious diseases are known in the art and the ordinary medical practitioner will be familiar with these laboratory tests which include but are not limited to microscopic analyses, cultivation dependent tests (such as cultures), and nucleic acid detection tests. These include wet mounts, stain-enhanced microscopy, immune microscopy (e.g., FISH), hybridization microscopy, particle agglutination, enzyme-linked immunosorbent assays, urine screening tests, DNA probe hybridization, serologic tests, etc. The medical practitioner will generally also take a full history and conduct a complete physical examination in addition to running the laboratory tests listed above.

A subject at risk of developing an infectious disease is one that is at risk of exposure to an infectious pathogen. Such subjects include those that live in an area where such pathogens are known to exist and where such infections are common. These subjects also include those that engage in high risk activities such as sharing of needles, engaging in unprotected sexual activity, routine contact with infected samples of subjects (e.g., medical practitioners), people who have undergone surgery, including but not limited to abdominal surgery, etc.

Formula I compounds are also indicated for treatment of human papillomavirus (HPV) infection. The current therapy for HPV is injection of IFN into a lesion and/or surgical ablation. A systemic treatment such as that envisioned for Formula I compounds would be desirable in comparison with current clinical therapies. Formula I compounds are similarly useful in combination with HPV vaccines currently in development such as HPV virus-like particle (VLP)-based vaccine (see, for example, Virology 2000 Jan. 20;266(2):237-45).

In still further aspects, the invention contemplates the use of Formula I compounds together with anti-microbial agents (e.g., anti-bacterial agents or anti-viral agents), for example, in order to reduce the risk of drug resistance by the microbial species, or for treatment following incidence of drug resistance.

The invention also contemplates the use of Formula I compounds together with antigens such as cancer antigens and microbial agents. Antigens associated with infectious diseases that can be used in the methods of the invention include whole bacteria, whole virus, whole fungi, whole parasites, and fragments thereof. Examples include non-infectious human papillomavirus-like particles (VLP) (which can be used as a cancer antigen as well, particularly for cervical cancer); and the like.

The invention intends to treat subjects that are not immunocompromised in some instances. Subject that are not immunocompromised (i.e., “non-immunocompromised”) are those that have blood cell counts in the normal range. Normal ranges of blood counts are known to the medical practitioner and reference can be made to a standard hematology textbook for such counts. In addition, reference can be made to published PCT application PCT/US00/14505. Non-immunocompromised subjects can include subjects that have not undergone any treatment that would render them immunocompromised. For example, such subjects may have a cancer but they have not undergone any treatment such as chemotherapy or radiation that would render them immunocompromised. Such subjects also would not inherently be immunocompromised as a result of the cancer. In some important embodiments, the subjects are at risk of developing an infection due to an impending surgical procedure, travel to a region where one or more infections are common, or they have experienced a skin abrasion, for example as a result of a trauma.

Thus, in one embodiment, the method intends to treat subjects free of symptoms calling for hemopoietic stimulation. Thus, the invention intends, in certain embodiments, to treat subjects at a time when they are free of symptoms requiring hemopoietic stimulating treatment or to treat subjects who have such symptoms with amounts or dosages or administration schedules that differ from those used to protect or restore normal or protective levels of hemopoietic cells. A subject who has previously experienced a need for hemopoietic stimulation but has since recovered its hemopoietic cells to normal or at least protective levels may still be treated by the methods described herein.

As used herein, the terms hemopoietic and hematopoietic are used interchangeably to mean all blood cells including myeloid and lymphoid cells. Myeloid cells include erythrocytes (i.e., red blood cells), macrophages, monocytes, granulocytes including neutrophils, eosinophils and basophils, mast cells, megakaryoctyes, platelets and dendritic cells, and lymphoid cells include T and B lymphocytes, thymic dendritic cells and natural killer (NK) cells. Hemopoietic stimulation, as used herein, refers to the increase in hemopoietic cell numbers or activity to normal or protective levels.

An example of a symptom calling for hemopoietic stimulation is hemopoietic cell numbers below normal or protective levels. A “normal” level as used herein may be a level in a control population, which preferably includes subjects having similar characteristics as the treated individual, such as age and sex. The “normal” level can also be a range, for example, where a population is used to obtain a baseline range for a particular group into which the subject falls. Thus, the “normal” value can depend upon a particular population selected. Preferably, the normal levels are those of apparently healthy subjects who have no prior history of hematopoietic cell disorders. Such “normal” levels, then can be established as preselected values, taking into account the category in which an individual falls. Appropriate ranges and categories can be selected with no more than routine experimentation by those of ordinary skill in the art. Either the mean or another preselected number within the range can be established as the normal preselected value.

In general, the normal range for neutrophils is about 1800-7250 per μl (mean −3650); for basophils 0-150 per μl (mean −30); for eosinophils 0-700 per μl (mean −150); for macrophages and monocytes 200-950 per μl (mean −430); for lymphocytes 1500-4000 per μl (mean −2500); for erythrocytes 4.2×10⁶-6.1×10⁶ per μl; and for platelets 112×10³-333×10³ per μl. The foregoing ranges are at the 95% confidence level.

In connection with certain conditions, the medical community has established certain preselected values. For example, mild neutropenia is characterized as having a count of between 1000 and 2000 per μl, moderate neutropenia at between 500 and 1000 per μl and severe neutropenia at below 500 per μl. Likewise, in adults, a lymphocyte count at less than 1500 is considered a medically undesirable condition. In children, the value is less than 3000. Other preselected values will be readily known to those of ordinary skill in the art.

A protective level of hematopoietic cells is the number of cells required to confer clinical benefit to the patient. The required level can be equal to or less than the “normal level”. Such levels are well known to those of ordinary skill in the art. For example, a protective level of neutrophils is above 1000, preferably, at least 1500.

Thus the methods of the invention, according to some embodiments, are directed towards subjects who possess normal or protective levels of hemopoietic cells, as described herein. Subjects with normal or protective levels of hemopoietic cells are considered to have normal hemopoietic activity. Likewise, in some embodiments, the invention is directed for use in subjects who are not immunocompromised. As used herein, the terms immunocompromised and immunosuppressed are used interchangeably. An example of an immunocompromised subject is one infected with HIV and experiencing AIDS-related symptoms such as low CD4+ T lymphocyte levels. In still other embodiments, the methods may be used in subjects who are HIV positive and who may be immunocompromised, provided that the agent is administered in an amount, a dosing regimen, and an administration schedule that have a therapeutic effect on abnormal proliferation, such as in a Kaposi's sarcoma tumor, but are not therapeutically effective in stimulating hemopoiesis in the subject.

According to still other embodiments, subjects of the invention are those who may have previously received anti-cancer therapy or who will in the future receive anti-cancer therapy but who do not at the time of treatment need hemopoietic stimulation, including a blood transfusion or administration of a hemopoietic stimulant such as a hemopoietic growth factor.

Thus in certain embodiments, the subjects are not myeloid or lymphoid suppressed or are not candidates for treatment with an agent which causes such suppression at the time of treatment with the methods of the instant invention. Myeloid suppressing conditions are those which induce a reduction in myeloid cells such as erythrocytes, neutrophils or platelets, to below protective or normal levels. Exemplary myelosuppressed conditions are hemopoietic malignancies, including leukemia and lymphoma and diseases such as chronic idiopathic neutropenia, cyclic neutropenia, anemia and thrombocytopenia. Similarly, lymphoid suppressing conditions are those which induce a reduction in lymphoid cells such as T lymphocytes. Suppression of lymphoid cells or some myeloid cells such as granulocytes is also referred to as immunosuppression since reduction in these cell types makes an individual susceptible to, inter alia, infection. Subjects may be exposed to myeloid, lymphoid or general immune suppressing conditions by the use of either immunosuppressant drugs such as cyclosporin or high dose chemotherapeutic compounds which affect dividing hemopoietic cells. Immuno-suppression may also arise as a result of treatment modalities such as total body irradiation or conditioning regimens prior to bone marrow transplantation. Viral infection, particularly as in the case of infection with human immunodeficiency virus (HIV), may also immunosuppress an individual. In some embodiments, subjects are those which have not been exposed and are not anticipated to be exposed to the above-mentioned conditions. In other embodiments, the instant invention aims to treat subjects who may have been myelosuppressed or immunosuppressed (e.g., by exposure to one or more of the above conditions), provided that at the time of treatment using the methods described herein, the subject has protective or normal levels of hemopoietic cells.

In still other embodiments, the invention aims to treat subjects who may exhibit symptoms calling for hemopoietic stimulation, provided that the agents are administered in doses, routes and schedules that would not result in hemopoietic stimulation, as explained below. In certain embodiments, the methods of the invention are not intended for use in the treatment of malignancies in HIV infected (i.e., HIV positive) subjects who have below normal or below protective levels of hemopoietic cells, unless the agents are used under conditions, such as administration routes, doses or dosing schedules, that are therapeutically effective in treating abnormal cell proliferation, as described herein, and not effective in stimulating hemopoiesis. For example, in some embodiments, the agent may be administered once a day, or twice a day, or three or more times a day, for more than 7 days, more than 10 days, more than 14 days or more than 20 days in order to achieve, for example, sustained desired systemic levels. In other embodiments, the agent may be given at timed intervals, such as, for example, every two days, every three days, every four days, every week or every two weeks. In still further embodiments, the agent may be delivered intravenously and continuously, for example, or by injection, such as, in single bolus administrations.

The agents of Formula I may be used together with other therapeutic agents to treat subjects. For example, the agent may be administered to a subject in combination with another anti-proliferative (e.g., an anti-cancer) therapy. As used herein, an anti-cancer therapy is a therapy effective in treating or preventing a cancer. Suitable anti-cancer therapies include surgical procedures to remove the tumor mass, chemotherapy or localization radiation. The other anti-proliferative therapy may be administered before, concurrent with, or after treatment with the agent of the invention. There may also be a delay of several hours, days and in some instances weeks between the administration of the different treatments, such that the agent may be administered before or after the other treatment.

As an example, the agent may be administered in combination with surgery to remove an abnormal proliferative cell mass. As used herein, “in combination with surgery” means that the agent may be administered prior to, during or after the surgical procedure. Surgical methods for treating cancer include intra-abdominal surgeries such as right or left hemicolectomy, sigmoid, subtotal or total colectomy and gastrectomy, radical or partial mastectomy, prostatectomy and hysterectomy. In these embodiments, the agent may be administered either by continuous infusion or in a single bolus. Administration during or immediately after surgery may include a lavage, soak or perfusion of the tumor excision site with a pharmaceutical preparation of the agent in a pharmaceutically acceptable carrier. In some embodiments, the agent is administered at the time of surgery as well as following surgery in order to inhibit the formation and development of metastatic lesions. The administration of the agent may continue for several hours, several days, several weeks, or in some instances, several months following a surgical procedure to remove a tumor mass.

The subjects can also be administered the agent in combination with non-surgical anti-proliferative (e.g., anti-cancer) drug therapy. Some anti-cancer agents can be categorized as DNA damaging agents and these include topoisomerase inhibitors (e.g., etoposide, ramptothecin, topotecan, teniposide, mitoxantrone), anti-microtubule agents (e.g., vincristine, vinblastine), anti-metabolic agents (e.g., cytarabine, methotrexate, hydroxyurea, 5-fluorouracil, floxuridine, 6-thioguanine, 6-mercaptopurine, fludarabine, pentostatin, chlorodeoxyadenosine), DNA alkylating agents (e.g., cisplatin, mechlorethamine, cyclophosphamide, ifosfamide, melphalan, chorambucil, busulfan, thiotepa, carmustine, lomustine, carboplatin, dacarbazine, procarbazine), DNA strand break inducing agents (e.g., bleomycin, doxorubicin, daunorubicin, idarubicin, mitomycin C), and radiation therapy.

Suitable anti-cancer compounds to be used in the invention include Acivicin; Aclarubicin; Acodazole Hydrochloride; Acronine; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Etoposide; Etoposide Phosphate; Etoprine; Fadrozole Hydrochloride; Fazarabine; Fenretinide; Floxuridine; Fludarabine Phosphate; Fluorouracil; Flurocitabine; Fosquidone; Fostriecin Sodium; Gemcitabine; Gemcitabine Hydrochloride; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-I a; Interferon Gamma-I b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safingol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Sulofenur; Talisomycin; Taxol; Taxotere; Tecogalan Sodium; Tegafur; Teloxantrone Hydrochloride; Temoporfin; Teniposide; Teroxirone; Testolactone; Thiamiprine; Thioguanine; Thiotepa; Tiazofurin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride.

Other anti-cancer drugs include: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; irinotecan; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anti cancer compound; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thalidomide; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene dichloride; topotecan; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer.

Anti-cancer supplementary potentiating compounds include: Tricyclic anti-depressant drugs (e.g., imipramine, desipramine, amitryptyline, clomipramine, trimipramine, doxepin, nortriptyline, protriptyline, amoxapine and maprotiline); non-tricyclic anti-depressant drugs (e.g., sertraline, trazodone and citalopram); Ca⁺⁺ antagonists (e.g., verapamil, nifedipine, nitrendipine and caroverine); Calmodulin inhibitors (e.g., prenylamine, trifluoroperazine and clomipramine); Amphotericin B; Triparanol analogues (e.g., tamoxifen); antiarrhythmic drugs (e.g., quinidine); antihypertensive drugs (e.g., reserpine); Thiol depleters (e.g., buthionine and sulfoximine) and multiple drug resistance reducing compounds such as Cremaphor EL.

Other compounds which are useful in combination therapy for the purpose of the invention include the antiproliferation compound, Piritrexim Isethionate; the antiprostatic hypertrophy compound, Sitogluside; the benign prostatic hyperplasia therapy compound, Tamsulosin Hydrochloride; the prostate growth inhibitor, Pentomone; radioactive compounds such as Fibrinogen I 125, Fludeoxyglucose F 18, Fluorodopa F 18, Insulin I 125, Insulin I 131, Iobenguane I 123, Iodipamide Sodium I 131, Iodoantipyrine I 131, Iodocholesterol I 131, Iodohippurate Sodium I 123, Iodohippurate Sodium I 125, Iodohippurate Sodium I 131, Iodopyracet I 125, Iodopyracet I 131, Iofetamine Hydrochloride I 123, Iomethin I 125, Iomethin I 131, Iothalamate Sodium I 125, Iothalamate Sodium I 131, Iotyrosine 1 131, Liothyronine I 125, Liothyronine I 131, Merisoprol Acetate Hg 197, Merisoprol Acetate Hg 203, Merisoprol Hg 197, Selenomethionine Se 75, Technetium Tc 99m Antimony Trisulfide Colloid, Technetium Tc 99m Bicisate, Technetium Tc 99m Disofenin, Technetium Tc 99m Etidronate, Technetium Tc 99m Exametazime, Technetium Tc 99m Furifosmin, Technetium Tc 99m Gluceptate, Technetium Tc 99m Lidofenin, Technetium Tc 99m Mebrofenin, Technetium Tc 99m Medronate, Technetium Tc 99m Medronate Disodium, Technetium Tc 99m Mertiatide, Technetium Tc 99m Oxidronate, Technetium Tc 99m Pentetate, Technetium Tc 99m Pentetate Calcium Trisodium, Technetium Tc 99m Sestamibi, Technetium Tc 99m Siboroxime, Technetium Tc 99m Succimer, Technetium Tc 99m Sulfur Colloid, Technetium Tc 99m Teboroxime, Technetium Tc 99m Tetrofosmin, Technetium Tc 99m Tiatide, Thyroxine I 125, Thyroxine I 131, Tolpovidone I 131, Triolein I 125 and Triolein I 131.

Particularly important anti-cancer agents are those selected from the group consisting of: annonaceous acetogenins; asimicin; rolliniastatin; guanacone, squamocin, bullatacin; squamotacin; taxanes; paclitaxel; gemcitabine; methotrexate FR-900482; FK-973; FR-66979; FK-317; 5-FU; FUDR; FdUMP; Hydroxyurea; Docetaxel; discodermolide; epothilones; vincristine; vinblastine; vinorelbine; meta-pac; irinotecan; SN-38; 10-OH campto; topotecan; etoposide; adriamycin; flavopiridol; Cis-Pt; carbo-Pt; bleomycin; mitomycin C; mithramycin; capecitabine; cytarabine; 2-Cl-2′ deoxyadenosine; Fludarabine-PO₄; mitoxantrone; mitozolomide; Pentostatin; Tomudex.

One particularly preferred class of anti-cancer agents are taxanes (e.g., paclitaxel and docetaxel). Another important category of anticancer agent is annonaceous acetogenin.

Other cancer therapies include hormonal manipulation, particularly for breast and gynecological cancers. Formula I compounds are also useful in combination with tamoxifen or aromatase inhibitor arimidex (i.e., anastrozole), or simply for disorders responsive to either (e.g., breast cancer).

Formula I compounds can also be combined, and/or administered substantially simultaneously, with enzyme inhibitor agents such as CDK inhibitors, tyrosine kinase inhibitors, MAP kinase inhibitors, and EGFR inhibitors (e.g., C225).

In important embodiments, the agents are administered together with anti-cancer compounds selected from the group consisting of aldesleukin, asparaginase, bleomycin sulfate, carboplatin, chlorambucil, cisplatin, cladribine, cyclophosphamide, cytarabine, dacarbazine, dactinomycin, daunorubicin hydrochloride, docetaxel, doxorubicin, doxorubicin hydrochloride, epirubicin hydrochloride, etoposide, etoposide phosphate, floxuridine, fludarabine, fluorouracil, gemcitabine, gemcitabine hydrochloride, hydroxyurea, idarubicin hydrochloride, ifosfamide, interferons, interferon-α2a, interferon-α2b, interferon-αn3, interferon-alb, interleukins, irinotecan, mechlorethamine hydrochloride, melphalan, mercatopurine, methotrexate, methotrexate sodium, mitomycin, mitoxantrone, paclitaxel, pegaspargase, pentostatin, prednisone, profimer sodium, procabazine hydrochloride, taxol, taxotere, teniposide, topotecan hydrochloride, vinblastine sulfate, vincristine sulfate and vinorelbine tartrate.

Certain methods and compositions comprise, in addition to the compounds of Formula I, an antibody or fragment thereof. The invention embraces the use of antibodies of all isotypes including IgM, IgA1, IgA2, sIgA, IgD, IgE, IgG1, IgG2, IgG3, and IgG4, having light chains that are either kappa or lambda chains.

The antibodies that can be used with the compounds of Formula I include those useful in cancer and infectious disease as well as other disorders for which antibodies and antigens have been identified and which would benefit from an enhanced immune response.

The antibodies or fragments thereof useful in the invention can be specific for any component of a particular target. Accordingly, the antibody can recognize and bind to proteins, lipids, carbohydrates, DNA, RNA, and any combination of these in molecular or supra-molecular structures (e.g., cell organelles such as mitochondria or ribosomes). The antibody or fragment thereof can also recognize a modification of the tumor cell, such as e.g., chemical modifications, or genetic modifications made by transfection ex vivo or in vivo with DNA or RNA. As used herein, the terms “antibody” and “immunoglobulin” are used interchangeably.

Bispecific antibodies can also be used in the invention. A bispecific antibody is one having one variable region that specifically recognizes a tumor antigen and the other variable region that specifically recognizes an antigenic epitope of a host immune effector cell that has lytic or growth inhibitory activity against the tumor. Bispecific and multispecific antibody complexes can be created by linkage of two or more immunoglobulins of different specificity for tumor antigens and/or effector cell antigens, either at the peptide or nucleic acid level.

Immunoglobulin can be produced in vivo in human or non-human species, or in vitro from immunoglobulin encoding DNA or cDNA isolated from libraries of DNA (e.g., phage display libraries). Immunoglobulin can also be modified genetically or chemically to incorporate human polypeptide sequences into non-human coding sequences (commonly referred to as humanization). Additionally, immunoglobulins can be modified chemically or genetically to incorporate protein, lipid, or carbohydrate moieties. Potential modifications could also include naturally occurring or synthetic molecular entities that are either directly toxic for tumor cells or serve as ligands or receptors for biologically active molecules that could suppress tumor growth. For example, growth factors, cytokines, chemokines and their respective receptors, immunologically active ligands or receptors, hormones or naturally occurring or synthetic toxins all represent biologically active molecules that could interact with suitably modified immunoglobulins and their targets.

As used herein, an “anti-cancer antibody or fragment thereof” is an antibody or an antibody fragment that binds to a cancer or tumor antigen.

The terms “cancer antigen” and “tumor antigen” are used interchangeably. A cancer antigen as used herein is a compound differentially associated with a tumor or cancer, preferably at the cell surface of a tumor or cancer cell, that is capable of invoking an immune response. The cancer antigen may be peptide in nature but it is not so limited. As an example, the antigen may be a lipid antigen, as described in U.S. patents U.S. Pat. No. 5,679,347, issued on Oct. 21, 1997 and U.S. Pat. No. 6,238,676 B1, issued on May 29, 2001. If the antigen is a peptide, then it invokes an immune response when it is presented (in a digested form) on the surface of an antigen presenting cell in the context of an MHC molecule. If the antigen is a lipid, then it invokes an immune response when it is presented in the context of a CD1 molecule. Cancer antigens can be prepared from cancer cells either by preparing crude extracts of cancer cells, for example, as described in Cohen, et al., 1994, Cancer Research, 54:1055, by partially purifying the antigens, by recombinant technology, or by de novo synthesis of known antigens. Cancer antigens include but are not limited to antigens that are recombinantly expressed, an immunogenic portion of, or a whole tumor or cancer. Such antigens can be isolated or prepared recombinantly or by any other means known in the art.

A cancer antigen encompasses antigens that are differentially expressed between cancer and normal cells. Due to this differential expression, these antigens can be targeted in anti-tumor therapies. Cancer antigens may be expressed in a regulated manner in normal cells. For example, they may be expressed only at certain stages of differentiation or at certain points in development of the organism or cell. Some are temporally expressed as embryonic and fetal antigens. Still others are never expressed in normal cells, or their expression in such cells is so low as to be undetectable.

Other cancer antigens are encoded by mutant cellular genes, such as oncogenes (e.g., activated ras oncogene), suppressor genes (e.g., mutant p53), fusion proteins resulting from internal deletions or chromosomal translocations. Still other cancer antigens can be encoded by viral genes such as those carried on RNA and DNA tumor viruses.

Examples of cancer antigens include HER 2 (p185), CD20, CD33, GD3 ganglioside, GD2 ganglioside, carcinoembryonic antigen (CEA), CD22, milk mucin core protein, TAG-72, Lewis A antigen, ovarian associated antigens such as OV-TL3 and MOv18, high Mr melanoma antigens recognized by antibody 9.2.27, HMFG-2, SM-3, B72.3, PR5C5, PR4D2, and the like. Other cancer antigens are described in U.S. Pat. No. 5,776,427. Still other cancer antigens are listed in Table 1.

Further examples include MAGE, MART-I/Melan-A, gp100, Dipeptidyl peptidase IV (DPPIV), adenosine deaminase-binding protein (ADAbp), FAP, cyclophilin b, Colorectal associated antigen (CRC)-C017-1A/GA733, Carcinoembryonic Antigen (CEA) and its immunogenic epitopes CAP-1 and CAP-2, etv6, aml1, Prostate Specific Antigen (PSA) and its immunogenic epitopes PSA-1, PSA-2, and PSA-3, prostate-specific membrane antigen (PSMA), T-cell receptor/CD3-zeta chain, MAGE-family of tumor antigens (e.g., MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-C5), GAGE-family of tumor antigens (e.g., GAGE-1, GAGE-2, GAGE-3, GAGE-4, GAGE-5, GAGE-6, GAGE-7, GAGE-8, GAGE-9), BAGE, RAGE, LAGE-1, NAG, GnT-V, MUM-1, CDK4, tyrosinase, p53, MUC family, HER2/neu, p21ras, RCAS1, α-fetoprotein, E-cadherin, α-catenin, β-catenin and γ-catenin, p120ctn, gp100^{Pmel117}, PRAME, NY-ESO-1, cdc27, adenomatous polyposis coli protein (APC), fodrin, Connexin 37, Ig-idiotype, p15, gp75, GM2 and GD2 gangliosides, viral products such as human papilloma virus proteins, Smad family of tumor antigens, Imp-1, P1A, EBV-encoded nuclear antigen (EBNA)-1, brain glycogen phosphorylase, SSX-1, SSX-2 (HOM-MEL-40), SSX-1, SSX-4, SSX-5, SCP-1 and CT-7, CD20 and c-erbB-2.

These antigens can be classified as indicated in Table 1.

TABLE 1
Classification of cancer antigens
TABLE 1a. Proteins encoded by genes that have undergone
chromosomal alteration in lymphoma and leukemia

| Genes | Disease |
|---|---|
| Activation of quiescent genes | |
| BCL-1 and IgH | Mantel cell lymphoma |
| BCL-2 and IgH | Follicular lymphoma |
| BCL-6 | Diffuse large B-cell lymphoma |
| TAL-1 and TCRδ or SIL | T-cell acute lymphoblastic |
| | leukemia |
| c-MYC and IgH or IgL | Burkitt lymphoma |
| MUN/IRF4 and IgH | Myeloma |
| PAX-5 (BSAP) | Immunocytoma |
| Creation of fusion genes | |
| RARα, PML, PLZF, NPM or NuMA | Acute promyelocytic leukemia |
| BCR and ABL | Chronic myeloid/acute |
| | lymphoblastic leukemia |
| MLL (HRX) | Acute leukemia |
| E2A and PBX or HLF | B-cell acute lymphoblastic |
| | leukemia |
| NPM, ALK | Anaplastic large cell leukemia |
| NPM, MLF-1 | Myelodysplastic syndrome/acute |
| | myeloid leukemia |

Adapted from Falini B. and Mason, D. Y. (2002) Blood 99: 409-426

TABLE 1b
Proteins specific to a tissue or cell lineage

| Protein | Disease |
|---|---|
| Cell-surface proteins | |
| CD20, CD22 | Non-Hodgkin's lymphoma, B-cell lymphoma, |
| | Chronic lymphocytic leukemia (CLL) |
| CD52 | B-cell CLL |
| CD33 | Acute myelogenous leukemia (AML) |
| CD10 (gp100) | Common (pre-B) acute lymphocytic leukemia |
| | and malignant melanoma |
| CD3/T-cell receptor (TCR) | T-cell lymphoma and leukemia |
| CD79/B-cell receptor (BCR) | B-cell lymphoma and leukemia |
| CD26 | Epithelial and lymphoid malignancies |
| Human leukocyte antigen (HLA)-DR, HLA- | Lymphoid malignancies |
| DP, and HLA-DQ | |
| RCAS1 | Gynecological carcinomas, bilary |
| | adenocarcinomas and ductal adenocarcinomas |
| | of the pancreas |
| Prostate specific membrane antigen | Prostate cancer |
| Epidermal growth factor receptors (high | |
| expression | |
| EGFR (HER1 or erbB1) and EGFRvIII | Brain, lung, breast, prostate and stomach |
| | cancer |
| erbB2 (HER2 or HER2/neu) | Breast cancer and gastric cancer |
| erbB3 (HER3) | Adenocarcinoma |
| erbB4 (HER4) | Breast cancer |
| Cell-associated proteins | |
| Tyrosinase, Melan-A/MART-1, tyrosinase | Malignant melanoma |
| related protein (TRP)-1/gp75 | |
| Polymorphic epithelial mucin (PEM) | Breast tumors |
| Human epithelial mucin (MUC1) | Breast, ovarian, colon and lung cancers |
| Secreted proteins | |
| Monoclonal immunoglobulin | Multiple myeloma and plasmacytoma |
| Immunoglobulin light chains | Multiple Myeloma |
| α-fetoprotein | Liver carcinoma |
| Kallikreins 6 and 10 | Ovarian cancer |
| Gastrin-releasing peptide/bombesin | Lung carcinoma |
| Prostate specific antigen | Prostate cancer |

TABLE 1c
Cancer testis (CT) antigens*
These antigens include MAGE-A1, -A3, -A6, -A12,
BAGE, GAGE, HAGE, LAGE-1, NY-ESO-1,
RAGE, SSX-1, -2, -3, -4, -5, -6, -7, -8, -9,
HOM-TES-14/SCP-1, HOM-TES-85 and PRAME.

| Protein | Disease |
|---|---|
| SSX-2, and -4 | Neuroblastoma |
| SSX-2 (HOM-MEL-40), MAGE, | Malignant melanoma |
| GAGE, BAGE and PRAME | |
| HOM-TES-14/SCP-1 | Meningioma |
| SSX-4 | Oligodendrioglioma |
| HOM-TES-14/SCP-1, MAGE-3 | Astrocytoma |
| and SSX-4 | |
| SSX member | Head and neck cancer, ovarian |
| | cancer, lymphoid tumors, |
| | colorectal cancer and |
| | breast cancer |
| RAGE-1, -2, -4, GAGE-1, -2, -3, | Head and neck squamous cell |
| -4, -5, -6, -7 and -8 | carcinoma (HNSCC) |
| HOM-TES14/SCP-1, SSX-1, | Non-Hodgkin's lymphoma |
| PRAME and CT-7 | |
| PRAME | Acute lymphoblastic |
| | leukemia (ALL), acute myelogenous |
| | leukemia (AML) and chronic |
| | lymphocytic leukemia (CLL) |

*These antigens are expressed in some normal tissues such as testis and in some cases placenta. Their expression is common in tumors of diverse lineages and as a group the antigens form targets for immunotherapy. Examples of tumor expression of CT antigens are as follows.

TABLE 1d
Proteins not-specific to a tissue or cell lineage*

Carcinoembryonic antigen (CEA) family: CD66a, CD66b, CD66c,
CD66d and CD66e.
*These antigens can be expressed in many different malignant tumors and can be targeted by immunotherapy.

TABLE 1e
Viral proteins

Human papilloma virus protein (cervical cancer)
EBV-encoded nuclear antigen (EBNA)-1 (lymphomas of neck and
oral cancer)

TABLE 1f
Mutated or aberrantly expressed molecules
CDK4 and beta-catenin in melanoma

Cancer or tumor antigens can also be classified according to the cancer or tumor they are associated with (i.e., expressed by). Cancers or tumors associated with tumor antigens include acute lymphoblastic leukemia (etv6; aml1; cyclophilin b), B cell lymphoma (Ig-idiotype); Burkitt's (Non-Hodgkin's) lymphoma (CD20); glioma (E-cadherin; α-catenin; β-catenin; γ-catenin; p120ctn), bladder cancer (p21ras), biliary cancer (p21ras), breast cancer (MUC family; HER2/neu; c-erbB-2), cervical carcinoma (p53; p21ras), colon carcinoma (p21ras; HER2/neu; c-erbB-2; MUC family), colorectal cancer (Colorectal associated antigen (CRC)—C017-1A/GA733; APC), choriocarcinoma (CEA), epithelial cell-cancer (cyclophilin b), gastric cancer (HER2/neu; c-erbB-2; ga733 glycoprotein), hepatocellular cancer (α-fetoprotein), Hodgkin's lymphoma (Imp-1; EBNA-1), lung cancer (CEA; MAGE-3; NY-ESO-1), lymphoid cell-derived leukemia (cyclophilin b), melanoma (p15 protein, gp75, oncofetal antigen, GM2 and GD2 gangliosides), myeloma (MUC family; p21ras), non-small cell lung carcinoma (HER2/neu; c-erbB-2), nasopharyngeal cancer (Imp-1; EBNA-1), ovarian cancer (MUC family; HER2/neu; c-erbB-2), prostate cancer (Prostate Specific Antigen (PSA) and its immunogenic epitopes PSA-1, PSA-2, and PSA-3; PSMA; HER2/neu; c-erbB-2), pancreatic cancer (p21ras; MUC family; HER2/neu; c-erbB-2; ga733 glycoprotein), renal (HER2/neu; c-erbB-2), squamous cell cancers of cervix and esophagus (viral products such as human papilloma virus proteins and non-infectious particles), testicular cancer (NY-ESO-1), T cell leukemia (HTLV-1 epitopes), and melanoma (Melan-A/MART-1; cdc27; MAGE-3; p21ras; gp100^{Pmel117}).

For examples of tumor antigens which bind to either or both MHC class I and MHC class II molecules, see the following references: Coulie, Stem Cells 13:393-403, 1995; Traversari et al., J. Exp. Med. 176:1453-1457, 1992; Chaux et al., J. Immunol. 163:2928-2936, 1999; Fujie et al., Int. J. Cancer 80:169-172, 1999; Tanzarella et al., Cancer Res. 59:2668-2674, 1999; van der Bruggen et al., Eur. J. Immunol. 24:2134-2140, 1994; Chaux et al., J. Exp. Med. 189:767-778, 1999; Kawashima et al, Hum. Immunol. 59:1-14, 1998; Tahara et al., Clin. Cancer Res. 5:2236-2241, 1999; Gaugler et al., J. Exp. Med. 179:921-930, 1994; van derBruggen et al., Eur. J. Immunol. 24:3038-3043, 1994; Tanaka et al., Cancer Res. 57:4465-4468, 1997; Oiso et al., Int. J. Cancer 81:387-394, 1999; Herman et al., Immunogenetics 43:377-383, 1996; Manici et al., J. Exp. Med. 189:871-876, 1999; Duffour et al., Eur. J. Immunol. 29:3329-3337, 1999; Zorn et al., Eur. J. Immunol. 29:602-607, 1999; Huang et al., J. Immunol. 162:6849-6854, 1999; Boel et al., Immunity 2:167-175, 1995; Van den Eynde et al., J. Exp. Med. 182:689-698, 1995; De Backer et al., Cancer Res. 59:3157-3165, 1999; Jager et al., J. Exp. Med. 187:265-270, 1998; Wang et al., J. Immunol. 161:3596-3606, 1998; Aarnoudse et al., Int. J. Cancer 82:442-448, 1999; Guilloux et al., J. Exp. Med. 183:1173-1183, 1996; Lupetti et al., J. Exp. Med. 188:1005-1016, 1998; Wolfel et al., Eur. J. Immunol. 24:759-764, 1994; Skipper et al., J. Exp. Med. 183:527-534, 1996; Kang et al., J. Immunol. 155:1343-1348, 1995; Morel et al., Int. J. Cancer 83:755-759, 1999; Brichard et al., Eur. J. Immunol. 26:224-230, 1996; Kittlesen et al., J. Immunol. 160:2099-2106, 1998; Kawakami et al., J. Immunol. 161:6985-6992, 1998; Topalian et al., J. Exp. Med. 183:1965-1971, 1996; Kobayashi et al., Cancer Research 58:296-301, 1998; Kawakami et al., J. Immunol. 154:3961-3968, 1995; Tsai et al., J. Immunol. 158:1796-1802, 1997; Cox et al., Science 264:716-719, 1994; Kawakami et al., Proc. Natl. Acad. Sci. USA 91:6458-6462, 1994; Skipper et al., J. Immunol. 157:5027-5033, 1996; Robbins et al., J. Immunol. 159:303-308, 1997; Castelli et al, J. Immunol. 162:1739-1748, 1999; Kawakami et al., J. Exp. Med. 180:347-352, 1994; Castelli et al., J. Exp. Med. 181:363-368, 1995; Schneider et al., Int. J. Cancer 75:451-458, 1998; Wang et al., J. Exp. Med. 183:1131-1140, 1996; Wang et al., J. Exp. Med. 184:2207-2216, 1996; Parkhurst et al., Cancer Research 58:4895-4901, 1998; Tsang et al., J. Natl Cancer Inst 87:982-990, 1995; Correale et al., J Natl Cancer Inst 89:293-300, 1997; Coulie et al., Proc. Natl. Acad. Sci. USA 92:7976-7980, 1995; Wolfel et al., Science 269:1281-1284, 1995; Robbins et al., J. Exp. Med. 183:1185-1192, 1996; Brandle et al., J. Exp. Med. 183:2501-2508, 1996; ten Bosch et al., Blood 88:3522-3527,1996; Mandruzzato et al., J. Exp. Med. 186:785-793, 1997; Gueguen et al., J. Immunol. 160:6188-6194, 1998; Gjertsen et al., Int. J. Cancer 72:784-790, 1997; Gaudin et al., J. Immunol. 162:1730-1738, 1999; Chiari et al., Cancer Res. 59:5785-5792, 1999; Hogan et al., Cancer Res. 58:5144-5150, 1998; Pieper et al., J. Exp. Med. 189:757-765, 1999; Wang et al., Science 284:1351-1354, 1999; Fisk et al., J. Exp. Med. 181:2109-2117, 1995; Brossart et al., Cancer Res. 58:732-736, 1998; Ropke et al., Proc. Natl. Acad. Sci. USA 93:14704-14707, 1996; Ikeda et al., Immunity 6:199-208, 1997; Ronsin et al., J. Immunol. 163:483-490, 1999; Vonderheide et al., Immunity 10:673-679,1999. These antigens as well as others are disclosed in PCT Application PCT/US98/18601.

In some preferred embodiments, the cancer antigen is VEGF, Anti-idiotypic mAb (GD3 ganglioside mimic), CD20, CD52, Anti-idiotypic mAb (CEA mimic), ERBB2, EGFR, CD22, ERBB2 X CD65 (fcγRI), EpCam, PEM and CD33.

Some commercially available anti-cancer antibodies along with their commercial source are as follows: anti-CD20 mAb (monoclonal antibody), rituximab, (Rituxan™, IDEC-Y2Bf), Rituxan™, Non-Hodgkin's lymphoma, B cell lymphoma (IDEC/Genentech); anti-CD20 mAb, tositumomab Bexxar, Non-Hodgkin's lymphoma (Corixa/GlaxoSmithKline); anti-HER2, trastuzumab, Herceptin™, breast and ovarian cancer (Genentech); anti-HER2, MDX-210, prostate, non-small cell lung cancer, breast, pancreatic, ovarian, renal and colon cancer (Medarex/Novartis); anti-CA125 mAb, oregovomab, B43.13, Ovarex™, ovarian cancer (Altarex); Breva-Rex, multiple myeloma, breast, lung, ovarian (Altarex); AR54, ovarian, brest, lung (Altarex); GivaRex, pancreas, stomach, colorectal (Altarex); ProstaRex, prostate (Altarex); anti-EGF receptor mAb, IMC-C225, Erbitux™, breast, head and neck, non-small cell lung, renal, prostate, colorectal and pancreatic cancer (ImClone Systems); anti-EGF receptor mAb, MDX-447, head and neck, prostate, lung, bladder, cervical, ovarian cancer (Medarex/Merck); gemtuzumab ozogamicin, Mylotarg, CMA-676, anti-CD33 (Wyeth Pharmaceuticals); anti-tissue factor protein (TF), (Sunol); ior-c5, colorectal cancer; ceal, colorectal cancer; c5, colorectal cancer; anti-EGF receptor mAb, MDX-447, head and neck, prostate, lung, bladder, cervical and ovarian cancer (Medarex/Merck); anti-17-1A mAb, edrecolomab, Panorex, colorectal, pancreatic, lung, breast and ovarian cancer (Centocor/Glaxo/Ajinomoto); anti-CD20 mAb (Y-90 labeled), ibritumomab tiuxetan (IDEC-Y2B8), Zevalin, Non-Hodgkin's lymphoma (IDEC); anti-idiotypic mAb mimic of ganglioside GD3 epitope, BEC2, small cell lung carcinoma, melanoma (ImClone Systems); anti-HLA-Dr10 mAb (131 ILYM-1), Oncolym™, Non-Hodgkin's lymphoma (Peregrine Pharmaceuticals); anti-CD33 humanized mAb (SMART M195), Zamyl™, acute myeloid leukemia, acute promyelocytic leukemia (Protein Design Labs); anti-CD52 humAb (LDP-03), CAMPATH, chronic lymphocytic leukemia (Millenium Pharmaceuticals/Ilex Oncology); anti-CD1 mAb, ior t6, cancer (Center of Molecular Immunology); anti-CAR (complement activating receptor) mAb, MDX-11, myeloid leukemia (Medarex); humanized bispecific mAb conjugates (complement cascade activators), MDX-22, myeloid leukemia (Medarex); OV103 (Y-90 labeled antibody), celogovab, OncoScint™, ovarian and prostate cancer (Cytogen); anti-171A mAb, 3622W94, non-small cell lung carcinoma, prostate cancer (Glaxo Wellcome plc); anti-VEGF (RhumAb-VEGF), bevacizumab, Avastin™, lung, breast, prostate, renal and colorectal cancer (Genentech); anti-TAC (IL-2 receptor) humanized Ab (SMART), daclizumab, Zenapax, leukemia, lymphoma (Protein Design Labs); anti-TAG-72 partially humanized bispecific Ab, MDX-220, lung, colon, prostate, ovarian, endometrial, pancreatic and gastric cancer (Medarex); anti-idiotypic mAb mimic of high molecular weight proteoglycan (I-Mel-1), MELIMMUNE-1, melanoma (IDEC); anti-idiotypic mAb mimic of high molecular weight proteoglycan (I-Mel-2), MELIMMUNE-2, melanoma (IDEC); anti-CEA Ab (hMN14), CEACide™, colorectal cancer and other cancers (Immunomedics); Pretarget™ radioactive targeting agents, cancer (NeoRx); hmAbH11 scFv fragment (NovomAb-G2), H11 scFv, cancer (Viventia Biotech); anti-DNA or DNA-associated proteins (histones) mAb and conjugates, TNT (e.g. Cotara™), cancer (Peregrine Pharmaceuticals); Gliomab-H mAb, brain cancer, melanoma, neuroblastoma (Viventia Biotech); GNI-250 mAb, colorectal cancer (Wyeth); anti-EGF receptor mAb, EMD-72000, cancer (Merck KgaA); anti-CD22 humanized Ab, LymphoCide, Non-Hodgkin's lymphoma (Immunomedics); anti-CD33 mAb conjugate with calicheamicin (CMA 676), gemtuzumab ozogamicin, Mylotarg™, acute myelogenous leukemia (Wyeth); Monopharm-C, colon, lung and pancreatic cancer (Viventia Biotech); anti-idiotypic human mAb to GD2 ganglioside, 4B5, melanoma, small-cell lung cancer, neuroblastoma (Viventia Biotech); anti-EGF receptor humanized Ab, ior egf/r3, cancers of epithelial origin (Center of Molecular Immunology); anti-ior c2 glycoprotein mAb, ior c5, colorectal and ovarian cancer (Center of Molecular Immunology); BABS (biosynthetic antibody binding site) proteins, breast cancer (Chiron); anti-FLK-2/FLT-3 mAb, cancer (tumor-associated angiogenesis) (ImClone Systems); mAb/small-molecule conjugate, TAP (tumor-activated prodrug), cancer (ImmunoGen); anti-GD-2 bispecific mAb, MDX-260, melanoma, glioma, neuroblastoma (Medarex); antinuclear autoantibodies (binds nucleosomes), ANA Ab, cancer (Procyon Biopharma); anti-HLA-DR Ab (SMART ID10 Ab), Remitogen™, Non-Hodgkin's B-cell lymphoma (Protein Design Labs); SMART ABL 364 Ab, epithelial cell cancers, breast, lung and colon cancer (Protein Design Labs/Novartis); anti-CEA I131-labeled mAb, ImmuRAIT-CEA, colorectal cancer (Immunomedics).

The antibody or antibody fragment provided herein can be used additionally for delivery of toxic substances to cancer cells. They may be conjugated (covalently or otherwise) to a toxin derived from plant, fungus, or bacteria. The toxin may be selected from the group consisting of A chain toxin, deglycosylated A chain toxin, ribosome inactivating protein, α-sarcin, aspergillin, restrictocin, ribonuclease, diptheria toxin, Pseudomonas exotoxin, calicheamicin, maytansinoids and ricin (e.g., from castor beans), but is not so limited. The antibody or antibody fragment may also conjugated to a chemotherapeutic agent, biological response modifiers, radioisotopes such as Iodine-131 and Yttrium-90 or those recited herein, or a cytotoxin. The chemotherapeutic agent may be selected from the group consisting of an anti-metabolite, an anthracycline, a vinca alkaloid, an antibiotic, an alkylating agent, and an epipodophyllotoxin, but is not so limited. The toxic substances can be concentrated in the desired region (e.g., a tumor) and non-specific toxicity to normal cells can be minimized. Antibodies to cancer antigens, vasculature, and microbial antigens can be modified in this manner. Antibodies to vasculature are particularly important because, generally, solid tumors are dependent upon newly formed blood vessels to survive. As a result, one strategy of many cancer medicaments is to attack the blood vessels feeding a tumor and/or the connective tissues (or stroma) supporting such blood vessels.

The invention embraces a number of classes of antibodies and fragments thereof including but not limited to antibodies directed to cancer antigens (as described above), cell surface molecule, stromal cell molecules, extracellular matrix molecules, and tumor vasculature associated molecules.

A cell surface molecule is a molecule that is expressed at the surface of a cell. In addition to an extracellular domain, it may further comprise a transmembrane domain and a cytoplasmic domain. Examples include HER 2, CD20, CD33, EGF receptor, HLA markers such as HLA-DR, CD52, CD1, CEA, CD22, GD2 ganglioside, FLK2/FLT3, VEGF, VEGFR, and the like.

A stromal cell molecule is a molecule expressed by a stromal cell. Examples include but are not limited to FAP and CD26.

An extracellular matrix molecule is a molecule found in the extracellular matrix. Examples include but are not limited to collagen, glycosaminoglycans (GAGs), proteoglycans, elastin, fibronectin and laminin.

A tumor vasculature associated molecule is a molecule expressed by vasculature of a tumor (i.e., a solid cancer rather than a systemic cancer such as leukemia). As with a cancer antigen, a tumor vasculature associated molecule may be expressed by normal vasculature however its presence on vasculature of a tumor makes it a suitable target for anti-cancer therapy. In some instances, the tumor vasculature associated molecule is expressed at a higher level in tumor vasculature than it is in normal vasculature. Examples include but are not limited to endoglin (see U.S. Pat. No. 5,660,827), ELAM-1, VCAM-1, ICAM-1, ligand reactive with LAM-1, MHC class II antigens, aminophospholipids such as phosphatidylserine and phosphatidylethanolamine (as described in U.S. Pat. No. 6,312,694), VEGFR1 (Fit-1) and VEGFR2 (KDR/Flk-1), and other tumor vasculature associated antigens such as those described in U.S. Pat. No. 5,776,427. Antibodies to endoglin are described in U.S. Pat. No. 5,660,827 and include TEC-4 and TEC-11, and antibodies that recognize identical epitopes to these antibodies. Antibodies to aminophospholipids are described in U.S. Pat. No. 6,312,694. Antibodies that inhibit VEGF are described in U.S. Pat. No. 6,342,219 and include 2C3 (ATCC PTA 1595). Other antibodies that are specific for tumor vasculature include antibodies that react to a complex of a growth factor and its receptor such as a complex of FGF and the FGFR or a complex of TGFβ and the TGFβR. Antibodies of this latter class are described in U.S. Pat. No. 5,965,132, and include GV39 and GV97.

In some preferred embodiments of the invention, the antibodies are Avastin (bevacizumab), BEC2 (mitumomab), Bexxar (tositumomab), Campath (alemtuzumab), CeaVac, Herceptin (trastuzumab), IMC-C225 (centuximab), LymphoCide (epratuzumab), MDX-210, Mylotarg (gemtuzumab ozogamicin), Panorex (edrecolomab), Rituxan (rituximab), Theragyn (pemtumomab), Zamyl, and Zevalin (ibritumomab tituxetan). The invention also covers antibody fragments thereof.

Other antibodies that can be used according to the invention include anti-TNFα antibody such as infliximab (Remicade) and etanercept (Enbrel) for rheumatoid arthritis and Crohn's disease palivizumab; anti-RSV antibody for pediatric subjects; bevacizumab, breast cancer; alemtuzumab, Campath-1H, breast and renal cancer, melanoma, B cell chronic lymphocytic leukemia (Millennium and ILEX); BLyS-mAb, fSLE and rheumatoid arthritis; anti-VEGF2, melanoma, breast cancer; anti-Trail receptor; B3 mAb, breast cancer; m170 mAb, breast cancer; mAB BR96, breast cancer; Abx-Cbl mAb, graft versus host disease.

It is to be understood that the antibodies embraced by the invention include those recited explicitly herein and also those that bind to the same epitope as those recited herein.

Also useful in the invention are antibodies such as the following, all of which are commercially available:

Apoptosis Antibodies:

BAX Antibodies: Anti-Human Bax Antibodies (Monoclonal), Anti-Human Bax Antibodies (Polyclonal), Anti-Murine Bax Antibodies (Monoclonal), Anti-Murine Bax Antibodies (Polyclonal);

Fas/Fas Ligand Antibodies: Anti-Human Fas/Fas Ligand Antibodies, Anti-Murine Fas/Fas Ligand Antibodies Granzyme Antibodies Granzyme B Antibodies;

BCL Antibodies: Anti Cytochrome C Antibodies, Anti-Human BCL Antibodies (Monoclonal), Anti-Human bcl Antibodies (Polyclonal), Anti-Murine bcl Antibodies (Monoclonal), Anti-Murine bcl Antibodies (Polyclonal);

Miscellaneous Apoptosis Antibodies: Anti TRADD, TRAIL, TRAFF, DR3 Antibodies Anti-Human Fas/Fas Ligand Antibodies Anti-Murine Fas/Fas Ligand Antibodies;

Miscellaneous Apoptosis Related Antibodies: BIM Antibodies: Anti Human, Murine bim Antibodies (Polyclonal), Anti-Human, Murine bim Antibodies (Monoclonal);

PARP Antibodies: Anti-Human PARP Antibodies (Monoclonal), Anti-Human PARP Antibodies (Polyclonal) Anti-Murine PARP Antibodies;

Caspase Antibodies: Anti-Human Caspase Antibodies (Monoclonal), Anti-Murine Caspase Antibodies;

Anti-CD Antibodies: Anti-CD29, PL18-5 PanVera, Anti-CD29, PL4-3 PanVera, Anti-CD41a, PT25-2 PanVera, Anti-CD42b, PL52-4 PanVera, Anti-CD42b, GUR20-5 PanVera, Anti-CD42b, WGA-3 PanVeraAnti-CD43, 1D4 PanVera, Anti-CD46, MCP75-6 PanVera, Anti-CD61, PL11-7 PanVera, Anti-CD61, PL8-5 PanVera, Anti-CD62/P-slctn, PL7-6 PanVera, Anti-CD62/P-slctn, WGA-1 PanVera, Anti-CD154, 5F3 PanVera; and anti-CD1, anti-CD2, anti-CD3, anti-CD4, anti-CD5, anti-CD6, anti-CD7, anti-CD8, anti-CD9, anti-CD10, anti-CD11, anti-CD 12, anti-CD13, anti-CD14, anti-CD15, anti-CD16, anti-CD17, anti-CD18, anti-CD19, anti-CD20, anti-CD21, anti-CD22, anti-CD23, anti-CD24, anti-CD25, anti-CD26, anti-CD27, anti-CD28, anti-CD29, anti-CD30, anti-CD31, anti-CD32, anti-CD33, anti-CD34, anti-CD35, anti-CD36, anti-CD37, anti-CD38, anti-CD39, anti-CD40 anti-CD41, anti-CD42, anti-CD43, anti-CD44, anti-CD45, anti-CD46, anti-CD47, anti-CD48, anti-CD49, anti-CD50, anti-CD51, anti-CD52, anti-CD53, anti-CD54, anti-CD55, anti-CD56, anti-CD57, anti-CD58, anti-CD59, anti-CD60, anti-CD61, anti-CD62, anti-CD63, anti-CD64, anti-CD65, anti-CD66, anti-CD67, anti-CD68, anti-CD69, anti-CD70, anti-CD71, anti-CD72, anti-CD73, anti-CD74, anti-CD75, anti-CD76, anti-CD77, anti-CD78, anti-CD79, anti-CD80, anti-CD81, anti-CD82, anti-CD83, anti-CD84, anti-CD85, anti-CD86, anti-CD87, anti-CD88, anti-CD89, anti-CD90, anti-CD91, anti-CD92, anti-CD93, anti-CD94, anti-CD95, anti-CD96, anti-CD97, anti-CD98, anti-CD99, anti-CD100, anti-CD101, anti-CD102, anti-CD103, anti-CD104, anti-CD105, anti-CD106, anti-CD107, anti-CD108, anti-CD109, anti-CD110, anti-CD111, anti-CD112, anti-CD113, anti-CD114, anti-CD115, anti-CD116, anti-CD117, anti-CD118, anti-CD119, anti-CD120, anti-CD121, anti-CD122, anti-CD123, anti-CD124, anti-CD125, anti-CD126, anti-CD127, anti-CD128, anti-CD129, anti-CD130, anti-CD131, anti-CD132, anti-CD133, anti-CD134, anti-CD135, anti-CD136, anti-CD137, anti-CD138, anti-CD139, anti-CD140, anti-CD141, anti-CD142, anti-CD143, anti-CD144, anti-CD145, anti-CD146, anti-CD147, anti-CD148, anti-CD149, anti-CD150, anti-CD151, anti-CD152, anti-CD153, anti-CD154, anti-CD155, anti-CD156, anti-CD157, anti-CD158, anti-CD159, anti-CD160, anti-CD161, anti-CD162, anti-CD163, anti-CD164, anti-CD165, anti-CD166, anti-CD167, anti-CD168, anti-CD169, anti-CD170, anti-CD171, anti-CD172, anti-CD173, anti-CD174, anti-CD175, anti-CD176, anti-CD177, anti-CD178, anti-CD179, anti-CD180, anti-CD181, anti-CD182, anti-CD183, anti-CD184, anti-CD185, anti-CD186, anti-CD187, anti-CD188, anti-CD189, anti-CD190, anti-CD191, anti-CD192, anti-CD193, anti-CD194, anti-CD195, anti-CD196, anti-CD197, anti-CD198, anti-CD199, anti-CD200, anti-CD201, anti-CD202, anti-CD203, anti-CD204, anti-CD205, anti-CD206, anti-CD207, anti-CD208, anti-CD209, anti-CD210, anti-CD211, anti-CD212, anti-CD213, anti-CD214, anti-CD215, anti-CD216, anti-CD217, anti-CD218, anti-CD219, anti-CD220, anti-CD221, anti-CD222, anti-CD223, anti-CD224, anti-CD225, anti-CD226, anti-CD227, anti-CD228, anti-CD229, anti-CD230, anti-CD231, anti-CD232, anti-CD233, anti-CD234, anti-CD235, anti-CD236, anti-CD237, anti-CD238, anti-CD239, anti-CD240 anti-CD241, anti-CD242, anti-CD243, anti-CD244, anti-CD245, anti-CD246, anti-CD247, anti-CD248, anti-CD249, anti-CD250, and the like.

Human Chemokine Antibodies: Human CNTF Antibodies, Human Eotaxin Antibodies, Human Epitherlial Neutrophil Activating Peptide-78, Human Exodus Antibodies, Human GRO Antibodies, Human HCC-1 Antibodies, Human 1-309 Antibodies, Human IP-10 Antibodies, Human 1-TAC Antibodies, Human LIF Antibodies, Human Liver-Expressed Chemokine Antibodies, Human lymphotoxin Antibodies, Human MCP Antibodies, Human MIP Antibodies, Human Monokine Induced by IFN-gamma Antibodies, Human NAP-2 Antibodies, Human NP-1 Antibodies, Human Platelet Factor-4 Antibodies, Human RANTES Antibodies, Human SDF Antibodies, Human TECK Antibodies;

Murine Chemokine Antibodies: Human B-Cell Attracting Murine Chemokine Antibodies, Chemokine-1 Antibodies, Murine Eotaxin Antibodies, Murine Exodus Antibodies, Murine GCP-2 Antibodies, Murine KC Antibodies, Murine MCP Antibodies, Murine MIP Antibodies, Murine RANTES Antibodies, Rat Chemokine Antibodies, Rat Chemokine Antibodies, Rat CNTF Antibodies, Rat GRO Antibodies, Rat MCP Antibodies, Rat MIP Antibodies, Rat RANTES Antibodies;

Cytokine/Cytokine Receptor Antibodies: Human Biotinylated Cytokine/Cytokine Receptor Antibodies, Human IFN Antibodies, Human IL Antibodies, Human Leptin Antibodies, Human Oncostatin Antibodies, Human TNF Antibodies, Human TNF Receptor Family Antibodies, Murine Biotinylated Cytokine/Cytokine Receptor Antibodies, Murine IFN Antibodies, Murine IL Antibodies, Murine TNF Antibodies, Murine TNF Receptor Antibodies; anti-CCR4 antibody;

Rat Cytokine/Cytokine Receptor Antibodies: Rat Biotinylated Cytokine/Cytokine Receptor Antibodies, Rat IFN Antibodies, Rat IL Antibodies, Rat TNF Antibodies;

ECM Antibodies: Collagen/Procollagen, Laminin, Collagen (Human), Laminin (Human), Procollagen (Human), Vitronectin/Vitronectin Receptor, Vitronectin (Human), Vitronectin Receptor (Human), Fibronectin/Fibronectin Receptor, Fibronectin (Human), Fibronectin Receptor (Human);

Growth Factor Antibodies: Human Growth Factor Antibodies, Murine Growth Factor Antibodies, Porcine Growth Factor Antibodies;

Miscellaneous Antibodies: Baculovirus Antibodies, Cadherin Antibodies, Complement Antibodies, Clq Antibodies, VonWillebrand Factor Antibodies, Cre Antibodies, HIV Antibodies, Influenza Antibodies, Human Leptin Antibodies, Murine Leptin Antibodies, Murine CTLA-4 Antibodies, Human CTLA-4 Antibodies, P450 Antibodies, RNA Polymerase Antibodies;

Neurobio Antibodies: Amyloid Antibodies, GFAP Antibodies, Human NGF Antibodies, Human NT-3 Antibodies, Human NT-4 Antibodies.

Still other antibodies can be used in the invention and these include antibodies listed in references such as the MSRS Catalog of Primary Antibodies, and Linscott's Directory.

The invention encompasses the use of both antibodies and antibody fragments. The antibodies may be monoclonal or polyclonal, and can be prepared by conventional methodology. They may further be isolated or present in an ascites fluid. Such antibodies can be further manipulated to create chimeric or humanized antibodies as will be discussed in greater detail below.

Significantly, as is well-known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). The pFc′ and Fc regions, for example, are effectors of the complement cascade but are not involved in antigen binding. An antibody from which the pFc′ region has been enzymatically cleaved, or which has been produced without the pFc′ region, designated an F(ab′)₂ fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDR3). The CDRs, and in particular the CDR3 regions, and more particularly the heavy chain CDR3, are largely responsible for antibody specificity.

It is now well-established in the art that the non-CDR regions of a mammalian antibody may be replaced with similar regions of co-specific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody. Thus, for example, PCT International Publication Number WO 92/04381 teaches the production and use of humanized murine RSV antibodies in which at least a portion of the murine FR regions has been replaced by FR regions of human origin. Such antibodies, including fragments of intact antibodies with antigen-binding ability, are often referred to as “chimeric” antibodies. Commercial sources of humanized or chimeric antibodies include GenPharm, Xenotech, AbGenix and CellGeneSys.

Thus, as will be apparent to one of ordinary skill in the art, the present invention also provides for F(ab′)₂, Fab, Fv and Fd fragments; chimeric antibodies in which the Fc and/or FR and/or CDR1 and/or CDR2 and/or light chain CDR3 regions have been replaced by homologous human or non-human sequences; chimeric F(ab′)₂ fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present invention also includes so-called single chain antibodies.

The Formula I compounds can be used in combination with various vaccines either currently being used or in development, whether intended for human or non-human subjects. Examples of vaccines for human subjects and directed to infectious diseases include the combined diphtheria and tetanus toxoids vaccine; pertussis whole cell vaccine; the inactivated influenza vaccine; the 23-valent pneumococcal vaccine; the live measles vaccine; the live mumps vaccine; live rubella vaccine; Bacille Calmette-Guerin (BCG) tuberculosis vaccine; hepatitis A vaccine; hepatitis B vaccine; hepatitis C vaccine; rabies vaccine (e.g., human diploid cell vaccine); inactivated polio vaccine; meningococcal polysaccharide vaccine; quadrivalent meningococcal vaccine; yellow fever live virus vaccine; typhoid killed whole cell vaccine; cholera vaccine; Japanese B encephalitis killed virus vaccine; adenovirus vaccine; cytomegalovirus vaccine; rotavirus vaccine; varicella vaccine; anthrax vaccine; small pox vaccine.

The vaccine methods and compositions described herein similarly envision the use of nucleic acid based vaccines in addition to peptide based vaccines. The art is familiar with nucleic acid based vaccines.

The invention seeks to enhance other forms of immunotherapy including dendritic cell vaccines. These vaccines generally include dendritic cells loaded ex vivo with antigens such as tumor-associated antigens. The dendritic cells can be incubated with the antigen, thereby allowing for antigen processing and expression on the cell surface, or the cells may simply be combined with the antigen prior to injection in vivo. Alternatively, the dendritic cells may be activated in vitro and then re-infused into a subject in the activated state. Formula I compounds can be combined with the dendritic cells in all of these embodiments. Examples of dendritic cell based vaccines include autologous tumour antigen-pulsed dendritic cells (advanced gynaecological malignancies); blood-derived dendritic cells loaded ex vivo with prostate cancer antigen (Provenge; Dendreon Corporation); blood-derived dendritic cells loaded ex vivo with antigen for multiple myeloma and other B-cell malignancies (Mylovenge; Dendreon Corporation); and blood-derived dendritic cells loaded ex vivo with antigen for cancers expressing the HER-2/neu proto-oncogene (APC8024; Dendreon Corporation); xenoantigen (e.g., PAP) loaded dendritic cells, and the like.

The Formula I compounds can also be used with normal and hyper-immune globulin therapy. Normal immune globulin therapy utilizes an antibody product from the serum of normal blood donors. This pooled product contains low titers of antibody to a wide range of antigens such as those of infectious pathogens (e.g., bacteria, viruses such as hepatitis A, parvovirus, enterovirus, fungi and parasites). Hyperimmune globulin therapy utilizes antibodies which are prepared from the serum of individuals who have high titers of an antibody to a particular antigen. The antibodies may be those that are currently used or in development for treating infectious diseases. Examples include zoster immune globulin (useful for the prevention of varicella-zoster in immunocompromised children and neonates), human rabies immunoglobulin (useful in the post-exposure prophylaxis of a subject bitten by a rabid animal), hepatitis A or B immune globulin (useful in the prevention of hepatitis A or B virus, especially in a subject exposed to the virus), RSV immune globulin (useful in the treatment of respiratory syncitial virus infections), tetanus immunoglobulin; measles immunoglobulin (useful in the prevention of infection in immunocompromised or adult subjects); rubella immunoglobulin (useful in the prevention of infection in pregnant female subjects).

Other antibodies for infectious diseases include anti-shiga toxin antibodies, anti-staphylococcal antibodies (Virion Systems), and the like.

In some embodiments, the compositions provided herein can further include other therapeutic agents such as anti-microbials agents, if the disease is an infectious disease. Examples of anti-microbials include anti-bacterials, anti-mycobacterials, anti-virals, anti-fungal, and anti-parasites.

Examples of anti-bacterials include β-lactam antibiotics, penicillins (such as natural penicillins, aminopenicillins, penicillinase-resistant penicillins, carboxy penicillins, ureido penicillins), cephalosporins (first generation, second generation, and third generation cephalosporins), and other β-lactams (such as imipenem, monobactams,), β-lactamase inhibitors, vancomycin, aminoglycosides and spectinomycin, tetracyclines, chloramphenicol, erythromycin, lincomycin, clindamycin, rifampin, metronidazole, polymyxins, sulfonamides and trimethoprim, and quinolines.

Anti-bacterials include: Acedapsone; Acetosulfone Sodium; Alamecin; Alexidine; Amdinocillin; Amdinocillin Pivoxil; Amicycline; Amifloxacin; Amifloxacin Mesylate; Amikacin; Amikacin Sulfate; Aminosalicylic acid; Aminosalicylate sodium; Amoxicillin; Amphomycin; Ampicillin; Ampicillin Sodium; Apalcillin Sodium; Apramycin; Aspartocin; Astromicin Sulfate; Avilamycin; Avoparcin; Azithromycin; Azlocillin; Azlocillin Sodium; Bacampicillin Hydrochloride; Bacitracin; Bacitracin Methylene Disalicylate; Bacitracin Zinc; Bambermycins; Benzoylpas Calcium; Berythromycin; Betamicin Sulfate; Biapenem; Biniramycin; Biphenamine Hydrochloride; Bispyrithione Magsulfex; Butikacin; Butirosin Sulfate; Capreomycin Sulfate; Carbadox; Carbenicillin Disodium; Carbenicillin Indanyl Sodium; Carbenicillin Phenyl Sodium; Carbenicillin Potassium; Carumonam Sodium; Cefaclor; Cefadroxil; Cefamandole; Cefamandole Nafate; Cefamandole Sodium; Cefaparole; Cefatrizine; Cefazaflur Sodium; Cefazolin; Cefazolin Sodium; Cefbuperazone; Cefdinir; Cefepime; Cefepime Hydrochloride; Cefetecol; Cefixime; Cefinenoxime Hydrochloride; Cefinetazole; Cefinetazole Sodium; Cefonicid Monosodium; Cefonicid Sodium; Cefoperazone Sodium; Ceforanide; Cefotaxime Sodium; Cefotetan; Cefotetan Disodium; Cefotiam Hydrochloride; Cefoxitin; Cefoxitin Sodium; Cefpimizole; Cefpimizole Sodium; Cefpiramide; Cefpiramide Sodium; Cefpirome Sulfate; Cefpodoxime Proxetil; Cefprozil; Cefroxadine; Cefsulodin Sodium; Ceftazidime; Ceftibuten; Ceftizoxime Sodium; Ceftriaxone Sodium; Cefuroxime; Cefuroxime Axetil; Cefuroxime Pivoxetil; Cefuroxime Sodium; Cephacetrile Sodium; Cephalexin; Cephalexin Hydrochloride; Cephaloglycin; Cephaloridine; Cephalothin Sodium; Cephapirin Sodium; Cephradine; Cetocycline Hydrochloride; Cetophenicol; Chloramphenicol; Chloramphenicol Palmitate; Chloramphenicol Pantothenate Complex; Chloramphenicol Sodium Succinate; Chlorhexidine Phosphanilate; Chloroxylenol; Chlortetracycline Bisulfate; Chlortetracycline Hydrochloride; Cinoxacin; Ciprofloxacin; Ciprofloxacin Hydrochloride; Cirolemycin; Clarithromycin; Clinafloxacin Hydrochloride; Clindamycin; Clindamycin Hydrochloride; Clindamycin Palmitate Hydrochloride; Clindamycin Phosphate; Clofazimine; Cloxacillin Benzathine; Cloxacillin Sodium; Cloxyquin; Colistimethate Sodium; Colistin Sulfate; Coumermycin; Coumermycin Sodium; Cyclacillin; Cycloserine; Dalfopristin; Dapsone; Daptomycin; Demeclocycline; Demeclocycline Hydrochloride; Demecycline; Denofungin; Diaveridine; Dicloxacillin; Dicloxacillin Sodium; Dihydrostreptomycin Sulfate; Dipyrithione; Dirithromycin; Doxycycline; Doxycycline Calcium; Doxycycline Fosfatex; Doxycycline Hyclate; Droxacin Sodium; Enoxacin; Epicillin; Epitetracycline Hydrochloride; Erythromycin; Erythromycin Acistrate; Erythromycin Estolate; Erythromycin Ethylsuccinate; Erythromycin Gluceptate; Erythromycin Lactobionate; Erythromycin Propionate; Erythromycin Stearate; Ethambutol Hydrochloride; Ethionamide; Fleroxacin; Floxacillin; Fludalanine; Flumequine; Fosfomycin; Fosfomycin Tromethamine; Fumoxicillin; Furazolium Chloride; Furazolium Tartrate; Fusidate Sodium; Fusidic Acid; Gentamicin Sulfate; Gloximonam; Gramicidin; Haloprogin; Hetacillin; Hetacillin Potassium; Hexedine; Ibafloxacin; Imipenem; Isoconazole; Isepamicin; Isoniazid; Josamycin; Kanamycin Sulfate; Kitasamycin; Levofuraltadone; Levopropylcillin Potassium; Lexithromycin; Lincomycin; Lincomycin Hydrochloride; Lomefloxacin; Lomefloxacin Hydrochloride; Lomefloxacin Mesylate; Loracarbef; Mafenide; Meclocycline; Meclocycline Sulfosalicylate; Megalomicin Potassium Phosphate; Mequidox; Meropenem; Methacycline; Methacycline Hydrochloride; Methenamine; Methenamine Hippurate; Methenamine Mandelate; Methicillin Sodium; Metioprim; Metronidazole Hydrochloride; Metronidazole Phosphate; Mezlocillin; Meziocillin Sodium; Minocycline; Minocycline Hydrochloride; Mirincamycin Hydrochloride; Monensin; Monensin Sodium; Nafcillin Sodium; Nalidixate Sodium; Nalidixic Acid; Natamycin; Nebramycin; Neomycin Palmitate; Neomycin Sulfate; Neomycin Undecylenate; Netilmicin Sulfate; Neutramycin; Nifuradene; Nifuraldezone; Nifuratel; Nifuratrone; Nifurdazil; Nifurimide; Nifurpirinol; Nifurquinazol; Nifurthiazole; Nitrocycline; Nitrofurantoin; Nitromide; Norfloxacin; Novobiocin Sodium; Ofloxacin; Ormetoprim; Oxacillin Sodium; Oximonam; Oximonam Sodium; Oxolinic Acid; Oxytetracycline; Oxytetracycline Calcium; Oxytetracycline Hydrochloride; Paldimycin; Parachlorophenol; Paulomycin; Pefloxacin; Pefloxacin Mesylate; Penamecillin; Penicillin G Benzathine; Penicillin G Potassium; Penicillin G Procaine; Penicillin G Sodium; Penicillin V; Penicillin V Benzathine; Penicillin V Hydrabamine; Penicillin V Potassium; Pentizidone Sodium; Phenyl Aminosalicylate; Piperacillin Sodium; Pirbenicillin Sodium; Piridicillin Sodium; Pirlimycin Hydrochloride; Pivampicillin Hydrochloride; Pivampicillin Pamoate; Pivampicillin Probenate; Polymyxin B Sulfate; Porfiromycin; Propikacin; Pyrazinamide; Pyrithione Zinc; Quindecamine Acetate; Quinupristin; Racephenicol; Ramoplanin; Ranimycin; Relomycin; Repromicin; Rifabutin; Rifametane; Rifamexil; Rifamide; Rifampin; Rifapentine; Rifaximin; Rolitetracycline; Rolitetracycline Nitrate; Rosaramicin; Rosaramicin Butyrate; Rosaramicin Propionate; Rosaramicin Sodium Phosphate; Rosaramicin Stearate; Rosoxacin; Roxarsone; Roxithromycin; Sancycline; Sanfetrinem Sodium; Sarmoxicillin; Sarpicillin; Scopafungin; Sisomicin; Sisomicin Sulfate; Sparfloxacin; Spectinomycin Hydrochloride; Spiramycin; Stallimycin Hydrochloride; Steffimycin; Streptomycin Sulfate; Streptonicozid; Sulfabenz; Sulfabenzamide; Sulfacetamide; Sulfacetamide Sodium; Sulfacytine; Sulfadiazine; Sulfadiazine Sodium; Sulfadoxine; Sulfalene; Sulfamerazine; Sulfameter; Sulfamethazine; Sulfamethizole; Sulfamethoxazole; Sulfamonomethoxine; Sulfamoxole; Sulfanilate Zinc; Sulfanitran; Sulfasalazine; Sulfasomizole; Sulfathiazole; Sulfazamet; Sulfisoxazole; Sulfisoxazole Acetyl; Sulfisoxazole Diolamine; Sulfomyxin; Sulopenem; Sultamicillin; Suncillin Sodium; Talampicillin Hydrochloride; Teicoplanin; Temafloxacin Hydrochloride; Temocillin; Tetracycline; Tetracycline Hydrochloride; Tetracycline Phosphate Complex; Tetroxoprim; Thiamphenicol; Thiphencillin Potassium; Ticarcillin Cresyl Sodium; Ticarcillin Disodium; Ticarcillin Monosodium; Ticlatone; Tiodonium Chloride; Tobramycin; Tobramycin Sulfate; Tosufloxacin; Trimethoprim; Trimethoprim Sulfate; Trisulfapyrimidines; Troleandomycin; Trospectomycin Sulfate; Tyrothricin; Vancomycin; Vancomycin Hydrochloride; Virginiamycin; Zorbamycin.

Anti-mycobacterials include Myambutol (Ethambutol Hydrochloride), Dapsone (4,4′-diaminodiphenylsulfone), Paser Granules (aminosalicylic acid granules), Priftin (rifapentine), Pyrazinamide, Isoniazid, Rifadin (Rifampin), Rifadin IV, Rifamate (Rifampin and Isoniazid), Rifater (Rifampin, Isoniazid, and Pyrazinamide), Streptomycin Sulfate and Trecator-SC (Ethionamide).

Anti-virals include amantidine and rimantadine, ribivarin, acyclovir, vidarabine, trifluorothymidine, ganciclovir, zidovudine, retinovir, and interferons.

Anti-virals further include: Acemannan; Acyclovir; Acyclovir Sodium; Adefovir; Alovudine; Alvircept Sudotox; Amantadine Hydrochloride; Aranotin; Arildone; Atevirdine Mesylate; Avridine; Cidofovir; Cipamfylline; Cytarabine Hydrochloride; Delavirdine Mesylate; Desciclovir; Didanosine; Disoxaril; Edoxudine; Enviradene; Enviroxime; Famciclovir; Famotine Hydrochloride; Fiacitabine; Fialuridine; Fosarilate; Foscarnet Sodium; Fosfonet Sodium; Ganciclovir; Ganciclovir Sodium; Idoxuridine; Kethoxal; Lamivudine; Lobucavir; Memotine Hydrochloride; Methisazone; Nevirapine; Penciclovir; Pirodavir; Ribavirin; Rimantadine Hydrochloride; Saquinavir Mesylate; Somantadine Hydrochloride; Sorivudine; Statolon; Stavudine; Tilorone Hydrochloride; Trifluridine; Valacyclovir Hydrochloride; Vidarabine; Vidarabine Phosphate; Vidarabine Sodium Phosphate; Viroxime; Zalcitabine; Zidovudine; Zinviroxime and integrase inhibitors.

Anti-fungals include imidazoles and triazoles, polyene macrolide antibiotics, griseofulvin, amphotericin B, and flucytosine. Antiparasites include heavy metals, antimalarial quinolines, folate antagonists, nitroimidazoles, benzimidazoles, avermectins, praxiquantel, ornithine decarboxylase inhbitors, phenols (e.g., bithionol, niclosamide); synthetic alkaloid (e.g., dehydroemetine); piperazines (e.g., diethylcarbamazine); acetanilide (e.g., diloxanide furonate); halogenated quinolines (e.g., iodoquinol (diiodohydroxyquin)); nitrofurans (e.g., nifurtimox); diamidines (e.g., pentamidine); tetrahydropyrimidine (e.g., pyrantel pamoate); sulfated naphthylamine (e.g., suramin).

Other anti-infectives include Difloxacin Hydrochloride; Lauryl Isoquinolinium Bromide; Moxalactam Disodium; Omidazole; Pentisomicin; Sarafloxacin Hydrochloride; Protease inhibitors of HIV and other retroviruses; Integrase Inhibitors of HIV and other retroviruses; Cefaclor (Ceclor); Acyclovir (Zovirax); Norfloxacin (Noroxin); Cefoxitin (Mefoxin); Cefuroxime axetil (Ceftin); Ciprofloxacin (Cipro); Aminacrine Hydrochloride; Benzethonium Chloride: Bithionolate Sodium; Bromchlorenone; Carbamide Peroxide; Cetalkonium Chloride; Cetylpyridinium Chloride: Chlorhexidine Hydrochloride; Clioquinol; Domiphen Bromide; Fenticlor; Fludazonium Chloride; Fuchsin, Basic; Furazolidone; Gentian Violet; Halquinols; Hexachlorophene: Hydrogen Peroxide; Ichthammol; Imidecyl Iodine; Iodine; Isopropyl Alcohol; Mafenide Acetate; Meralein Sodium; Mercufenol Chloride; Mercury, Ammoniated; Methylbenzethonium Chloride; Nitrofurazone; Nitromersol; Octenidine Hydrochloride; Oxychlorosene; Oxychlorosene Sodium; Parachlorophenol, Camphorated; Potassium Permanganate; Povidone-Iodine; Sepazonium Chloride; Silver Nitrate; Sulfadiazine, Silver; Symclosene; Thimerfonate Sodium; Thimerosal: Troclosene Potassium.

The invention also embraces the use of adjuvants. Adjuvant substances derived from microorganisms, such as bacillus Calmette-Guerin, heighten the immune response and enhance resistance to tumors in animals. Adjuvants that may be combined with the compounds of Formula I include alum, immunostimulatory oligonucleotides such as CpG oligonucleotides, QS-21, and the like. These and other adjuvants are listed herein in greater detail. Other therapeutic agents include but are not limited to nucleic acid adjuvants, non-nucleic acid adjuvants, cytokines, non-immunotherapeutic antibodies, antigens, etc.

A “nucleic acid adjuvant” is an adjuvant that is a nucleic acid. Examples include immunostimulatory nucleic acid molecules such as those containing CpG dinucleotides, as described in U.S. patents U.S. Pat. No. 6,194,388B1, issued Feb. 27, 2001, U.S. Pat. No. 6,207,646 B1, issued Mar. 27, 2001, and U.S. Pat. No. 6,239,116 B1, issued May 29, 2001.

A “non-nucleic acid adjuvant” is any molecule or compound except for the immunostimulatory nucleic acids described herein which can stimulate the humoral and/or cellular immune response. Non-nucleic acid adjuvants include, for instance, adjuvants that create a depo effect, immune-stimulating adjuvants, adjuvants that create a depo effect and stimulate the immune system and mucosal adjuvants.

An “adjuvant that creates a depo effect” as used herein is an adjuvant that causes an antigen, such as a cancer antigen present in a cancer vaccine, to be slowly released in the body, thus prolonging the exposure of immune cells to the antigen. This class of adjuvants includes but is not limited to alum (e.g., aluminum hydroxide, aluminum phosphate); or emulsion-based formulations including mineral oil, non-mineral oil, water-in-oil or oil-in-water-in oil emulsion, oil-in-water emulsions such as Seppic ISA series of Montanide adjuvants (e.g., Montanide ISA 720, AirLiquide, Paris, France); MF-59 (a squalene-in-water emulsion stabilized with Span 85 and Tween 80; Chiron Corporation, Emeryville, Calif.; and PROVAX (an oil-in-water emulsion containing a stabilizing detergent and a micelle-forming agent; IDEC Pharmaceuticals Corporation, San Diego, Calif.).

An “immune stimulating adjuvant” is an adjuvant that causes activation of a cell of the immune system. It may, for instance, cause an immune cell to produce and secrete cytokines. This class of adjuvants includes but is not limited to saponins purified from the bark of the Q. saponaria tree, such as QS21 (a glycolipid that elutes in the 21^{st} peak with HPLC fractionation; Antigenics, Inc., Waltham, Mass.); poly [di (carboxylatophenoxy) phosphazene (PCPP polymer; Virus Research Institute, USA); derivatives of lipopolysaccharides such as monophosphoryl lipid A (MPL; Ribi ImmunoChem Research, Inc., Hamilton, Mont.), muramyl dipeptide (MDP; Ribi) and threonyl-rnuramyl dipeptide (t-MDP; Ribi); OM-174 (a glucosamine disaccharide related to lipid A; OM Pharma SA, Meyrin, Switzerland); and Leishmania elongation factor (a purified Leishmania protein; Corixa Corporation, Seattle, Wash.).

“Adjuvants that create a depo effect and stimulate the immune system” are those compounds which have both of the above-identified functions. This class of adjuvants includes but is not limited to ISCOMS (Immunostimulating complexes which contain mixed saponins, lipids and form virus-sized particles with pores that can hold antigen; CSL, Melbourne, Australia); SB-AS2 (SmithKline Beecham adjuvant system #2 which is an oil-in-water emulsion containing MPL and QS21: SmithKline Beecham Biologicals [SBB], Rixensart, Belgium); SB-AS4 (SmithKline Beecham adjuvant system #4 which contains alum and MPL; SBB, Belgium); non-ionic block copolymers that form micelles such as CRL 1005 (these contain a linear chain of hydrophobic polyoxpropylene flanked by chains of polyoxyethylene; Vaxcel, Inc., Norcross, Ga.); and Syntex Adjuvant Formulation (SAF, an oil-in-water emulsion containing Tween 80 and a nonionic block copolymer; Syntex Chemicals, Inc., Boulder, Colo.).

A “non-nucleic acid mucosal adjuvant” as used herein is an adjuvant other than an immunostimulatory nucleic acid that is capable of inducing a mucosal immune response in a subject when administered to a mucosal surface in conjunction with an antigen. Mucosal adjuvants include but are not limited to Bacterial toxins: e.g., Cholera toxin (CT), CT derivatives including but not limited to CT B subunit (CTB) (Wu et al., 1998, Tochikubo et al., 1998); CTD53 (Val to Asp) (Fontana et al., 1995); CTK97 (Val to Lys) (Fontana et al., 1995); CTK104 (Tyr to Lys) (Fontana et al., 1995); CTD53/K63 (Val to Asp, Ser to Lys) (Fontana et al., 1995); CTH54 (Arg to His) (Fontana et al., 1995); CTN 107 (His to Asn) (Fontana et al., 1995); CTE 114 (Ser to Glu) (Fontana et al., 1995); CTE112K (Glu to Lys) (Yamamoto et al., 1997a); CTS61F (Ser to Phe) (Yamamoto et al., 1997a, 1997b); CTS106 (Pro to Lys) (Douce et al., 1997, Fontana et al., 1995); andCTK63 (Ser to Lys) (Douce et al., 1997, Fontana et al., 1995), Zonula occludens toxin, zot, Escherichia coli heat-labile enterotoxin, Labile Toxin (LT), LT derivatives including but not limited to LT B subunit (LTB) (Verweij et al., 1998); LT7K (Arg to Lys) (Komase et al., 1998, Douce et al., 1995); LT61F (Ser to Phe) (Komase et al., 1998); LT112K (Glu to Lys) (Komase et al., 1998); LT118E (Gly to Glu) (Komase et al., 1998); LT146E (Arg to Glu) (Komase et al., 1998); LT192G (Arg to Gly) (Komase et al., 1998); LTK63 (Ser to Lys) (Marchetti et al., 1998, Douce et al., 1997, 1998, Di Tommaso et al., 1996); and LTR72 (Ala to Arg) (Giuliani et al., 1998), Pertussis toxin, PT. (Lycke et al., 1992, Spangler BD, 1992, Freytag and Clemments, 1999, Roberts et al., 1995, Wilson et al., 1995) including PT-9K/129G (Roberts et al., 1995, Cropley et al., 1995); Toxin derivatives (see below) (Holmgren et al., 1993, Verweij et al., 1998, Rappuoli et al., 1995, Freytag and Clements, 1999); Lipid A derivatives (e.g., monophosphoryl lipid A, MPL) (Sasaki et al., 1998, Vancott et al., 1998; Muramyl Dipeptide (MDP) derivatives (Fukushima et al., 1996, Ogawa et al., 1989, Michalek et al., 1983, Morisaki et al., 1983); Bacterial outer membrane proteins (e.g., outer surface protein A (OspA) lipoprotein of Borrelia burgdorferi, outer membrane protine of Neisseria meningitidis) (Marinaro et al., 1999, Van de Verg et al., 1996); Oil-in-water emulsions (e.g., MF59) (Barchfield et al., 1999, Verschoor et al., 1999, O'Hagan, 1998); Aluminum salts (Isaka et al., 1998, 1999); and Saponins (e.g., QS21) Aquila Biopharmaceuticals, Inc., Worcester, Mass.) (Sasaki et al., 1998, MacNeal et al., 1998), ISCOMS, MF-59 (a squalene-in-water emulsion stabilized with Span 85 and Tween 80; Chiron Corporation, Emeryville, Calif.); the Seppic ISA series of Montanide adjuvants (e.g., Montanide ISA 720; AirLiquide, Paris, France); PROVAX (an oil-in-water emulsion containing a stabilizing detergent and a micell-forming agent; IDEC Pharmaceuticals Corporation, San Diego, Calif.); Syntext Adjuvant Formulation (SAF; Syntex Chemicals, Inc., Boulder, Colo.); poly[di(carboxylatophenoxy)phosphazene (PCPP polymer; Virus Research Institute, USA) and Leishmania elongation factor (Corixa Corporation, Seattle, Wash.).

Cytokines and chemokines can potentially be cleaved and thereby inactivated by post proline cleaving enzymes. Administration of Formula I compounds with cytokines and/or chemokines can enhance the efficacy of these latter agents by protecting them from degradation.

Immune responses can also be induced or augmented by the co-administration or co-linear expression of cytokines or chemokines (Bueler & Mulligan, 1996; Chow et al., 1997; Geissler et al., 1997; Iwasaki et al., 1997; Kim et al., 1997) or B-7 co-stimulatory molecules (Iwasaki et al., 1997; Tsuji et al., 1997) with the Formula I compounds and anti-cancer antibodies. The cytokines and/or chemokines can be administered directly or may be administered in the form of a nucleic acid vector that encodes the cytokine, such that the cytokine can be expressed in vivo. In one embodiment, the cytokine or chemokine is administered in the form of a plasmid expression vector. The term “cytokine” is used as a generic name for a diverse group of soluble proteins and peptides which act as humoral regulators at nano- to picomolar concentrations and which, either under normal or pathological conditions, modulate the functional activities of individual cells and tissues. These proteins also mediate interactions between cells directly and regulate processes taking place in the extracellular environment. Cytokines also are central in directing the T cell response.

Examples of cytokines include, but are not limited to IL-1, IL-2, IL-4, IL-5, IL-6, IL-7, IL-10, IL-12, IL-15, IL-18, granulocyte-macrophage colony stimulating factor (GM-CSF), granulocyte colony stimulating factor (G-CSF), interferon-γ (IFN-γ), IFN-α, tumor necrosis factor (TNF), TGF-β, FLT-3 ligand, and CD40 ligand. In some embodiments, the cytokine is a Th1 cytokine. In still other embodiments, the cytokine is a Th2 cytokine.

The term “chemokine” is used as a generic name for peptides or polypeptides that act principally to chemoattract effector cells of both innate and adaptive immunity. Chemokines are thought to coordinate immunological defenses against tumors and infectious agents by concentrating neutrophils, macrophages, eosinophils and T and B lymphocytes at the anatomical site in which the tumor or infectious agent is present. In addition, many chemokines are known to activate the effector cells so that their immune functions (e.g., cytolysis of tumor cells) are enhanced on a per cell basis. Two groups of chemokines are distinguished according to the positions of the first two cysteine residues that are conserved in the amino-terminal portions of the polypeptides. The residues can either be adjacent or separated by one amino acid, thereby defining the CC and CXC cytokines respectively. The activity of each chemokine is restricted to particular effector cells, and this specificity results from a cognate interaction between the chemokine and a specific cell membrane receptor expressed by the effector cells. For example, the CXC chemokines IL-8, Groα/β and ENA 78 act specifically on neutrophils, whereas the CC chemokines RANTES, MIP-1α and MCP-3 act on monocytes and activated T cells. In addition, the CXC chemokine IP-10 appears to have anti-angiogenic activity against tumors as well as being a chemoattractant for activated T cells. MIP-1α also reportedly has effects on hemopoietic precursor cells.

The Formula I compound and the other therapeutic agent(s) may be administered simultaneously or sequentially. When the other therapeutic agents are administered simultaneously they can be administered in the same or separate formulations, but are at least administered at the same time (e.g., within minutes of each other). The administration of the other therapeutic agents (such as adjuvants) and the Formula I compounds can also be temporally separated, meaning that the therapeutic agents are administered at a different time, either before or after, the administration of the Formula I compounds. The separation in time between the administration of these compounds may be a matter of minutes or it may be longer. Agents of Formula I, adjuvants and yet other therapeutic agents can be used together as well.

In some embodiments, the agents of Formula I are administered daily for more than 7 days, more than 10 days, more than 14 days or more than 20 days. In still other embodiments, the agent is administered over a period of weeks, or months. In still other embodiments, the agent is delivered on alternate days. For example, the agent is delivered every two days, every three days, every four days, every five days, every six days, every week, or every month.

According to the methods of the invention, the agents of Formula I may be administered prior to, concurrent with, or following other treatment (e.g., anti-cancer compounds, surgery or radiation). The administration schedule may involve administering the different agents in an alternating fashion. In other embodiments, the agent may be delivered before and during, or during and after, or before and after treatment with other therapies. In some cases, the agent is administered more than 24 hours before the administration of other treatment. In other embodiments, more than one therapy may be administered to a subject. For example, the subject may receive the agents of the invention, in combination with both surgery and at least one other anti-cancer compound. As another example, the agent may be administered in combination with more than one anti-cancer drug.

In methods particularly directed at subjects at risk of developing a disorder, timing of the administration of the agent of Formula I and the other therapeutic agent may be particularly important. For instance, in a subject with a genetic predisposition to cancer, the agents may be administered to the subject on a routine schedule.

A “routine schedule” as used herein, refers to a predetermined designated period of time. The routine schedule may encompass periods of time which are identical or which differ in length, as long as the schedule is predetermined. For instance, the routine schedule may involve administration on a daily basis, every two days, every three days, every four days, every five days, every six days, a weekly basis, a monthly basis or any set number of days or weeks there-between, every two months, three months, four months, five months, six months, seven months, eight months, nine months, ten months, eleven months, twelve months, etc. Alternatively, the predetermined routine schedule may involve administration on a daily basis for the first week, followed by a monthly basis for several months, and then every three months after that. Any particular combination would be covered by the routine schedule as long as it is determined ahead of time that the appropriate schedule involves administration on a certain day.

In some important embodiments, the timing of administration of the Formula I compound and the antigen are important. Thus, the invention embraces the administration of a Formula I compound, preferably with an antigen, prior to treatment with other conventional therapy. For example, if the subject has cancer, then conventional therapy includes surgical removal of a tumor, radiation therapy, or chemotherapy. It is preferred in some instances to administer the Formula I compound with antigen prior to this therapy, and even more preferred to administer the Formula I compound with antigen after this therapy as well. Thus, the method would involve both a prime and a boost dose to antigen (with the Formula I compound). In some embodiments, the antigen alone can be administered particularly in the boost dose.

In embodiments involving the administration of Formula I agents and an antibody such as the anti-HER2 antibody trastuzumab (Herceptin™), the antibody may be administered initially in a dose of 4 mg/kg (dose/unit body weight) as a 90 minute infusion followed by a weekly maintenance dose of 2 mg/kg. In embodiments involving the administration of Formula I agents and an antibody such as the anti-CD20 antibody rituximab (Rituxan™), the antibody may be administered in weekly infusions for 4 or 8 doses (i.e., for 4-8 weeks), each dose being 375 mg/m² (dose/unit body surface area). Formula I compounds could be administered, twice daily, for a period immediately prior to the initial antibody dose (e.g., 7 days). Since Formula I compounds will expand immune effector cells (e.g., neutrophils, macrophages, eosinophils and T lymphocytes) and direct them to the microenvironment of the tumor, pretreatment with such compounds will accelerate cytotoxicity mediated by the subsequent administration of antibody. Thus, Formula I compounds can be used solely in a pretreatment regime (i.e., prior to exposure to the antibody), or in a combination of pre- and post-treatment administrations. As a non-limiting example of this latter embodiment, pre-treatment with a Formula I compound can be followed by subsequent courses of defined period (e.g., 7 days) administration that could either be concurrent or spaced by intervals (e.g., 7 day pretreatment, 7 day gap, 7 day treatment etc.). Antibody treatment would be continue weekly as currently recommended by the manufacturer (e.g., Genentech, Inc., IDEC Pharmaceuticals, etc.).

The antibody or antibody fragment may be administered together with the agent of Formula I in a multi-day cycle. The multi-day cycle be a 2, 3, 4, 5, 6, 7, 8, 9, 10, or more day cycle. The antibody or fragment thereof may be administered on the first day of such a cycle, followed by administration of the Formula I agent for a number of days, which may or may not be consecutive. For example, the Formula I agent may be administered on all remaining days of a multi-day cycle. The Formula I agent may be administered once, twice, thrice, or more times per day as well. The multi-day cycle may be repeated once, twice, thrice, or more times. Alternatively, it may be repeated for a length of time such as a week, a month, two months, or more, depending upon the status of the subject and the therapeutic response observed. As an non-limiting example, the antibody or fragment thereof is administered on the first day of a seven day cycle, and the Formula I agent is administered twice a day for the remaining six days of the seven day cycle. The seven day cycle is performed four times resulting in a 28 day treatment.

If the agent of Formula I is administered together with a chemotherapeutic, then it may be desirable in some embodiments to administer the agent of Formula I more frequently than the other therapeutic agent. For example, the agent of Formula I may be administered daily or twice daily and the other therapeutic agent may be administered once every 2, 3, 4, 5, 6 or 7 days, or every 1, 2, 3 or 4 weeks. The agent of Formula I may also be administered less frequently than daily.

The agents of the invention are administered in effective amounts. An effective amount is a dosage of the agent sufficient to provide a medically desirable result. The effective amount will vary with the particular condition being treated, the age and physical condition of the subject being treated, the severity of the condition, the duration of the treatment, the nature of the concurrent or combination therapy (if any), the specific route of administration (e.g., as in the present invention, oral administration or administration by injection) and like factors within the knowledge and expertise of the health practitioner. The dosage may be adjusted by the individual physician or veterinarian, particularly in the event of any complication. It is preferred generally that a maximum dose be used, that is, the highest safe dose according to sound medical judgment. In general, a therapeutically effective amount means that amount necessary to delay the onset of, inhibit the progression of, or halt altogether the particular condition being treated.

Treatment after a disorder has started aims to reduce, ameliorate or altogether eliminate the disorder, and/or its associated symptoms, or prevent it from becoming worse. Treatment of subjects before a disorder has started (i.e., prophylactic treatment) aims to reduce the risk of developing the disorder. As used herein, the term “prevent” refers to the prophylactic treatment of patients who are at risk of developing a disorder (resulting in a decrease in the probability that the subject will develop the disorder), and to the inhibition of further development of an already established disorder.

For example, in connection with methods directed towards treating subjects having a condition characterized by abnormal mammalian cell proliferation, an effective amount to inhibit proliferation would be an amount sufficient to reduce or halt altogether the abnormal mammalian cell proliferation so as to slow or halt the development of or the progression of a cell mass such as, for example, a tumor. As used in the embodiments, “inhibit” embraces all of the foregoing. In other embodiments, a effective amount will be an amount necessary to extend the dormancy of micrometastases or to stabilize any residual primary tumor cells following surgical or drug therapy.

In still other embodiments, the agent is delivered in an amount, a dose, and a schedule which is therapeutically effective in inhibiting proliferation yet which is not therapeutically effective in stimulating hemopoiesis in the subject. In administering the agents of the invention to subjects, dosing amounts, dosing schedules, routes of administration and the like can be selected so as to affect the other known activities of these compounds. For example, amounts, dosing schedules and routes of administration can be selected as described below, whereby therapeutically effective levels for inhibiting proliferation are provided, yet therapeutically effective levels for restoring hemopoietic deficiency are not provided. As another example, local administration to tumors or protected body areas such as the brain may result in therapeutically effective levels for inhibiting proliferation, but may be non-therapeutically effective levels for hemopoietic cell stimulation.

In addition, agents of Formula I can be selected that are effective as anti-proliferative agents but are relatively ineffective as hemopoietic cell stimulatory or activating agents. Thus, certain subjects who require both hemopoietic stimulation and/or activation and proliferation inhibition may be treated with different agents of Formula I simultaneously, one each for the desired therapeutic effect, or with a single agent but in different dosages, schedules, and/or route to achieve both hemopoietic stimulation and proliferation inhibition at therapeutic levels.

Generally, an effective amount typically varies from 0.01 mg/kg to about 1000 mg/kg, preferably from about 0.1 mg/kg to about 200 mg/kg, and most preferably from about 0.2 mg/kg to about 20 mg/kg, in one or more dose administrations daily, for one or more days.

In particularly important embodiments, the agent is administered in amounts of less than or equal to 1.0 mg/kg per day. This includes amounts equal to or less than 0.9, 0.8, 0.7, 0.6, 0.5, 0.4, 0.3, 0.2, 0.1 mg/kg per day. The agents may also be administered in amounts of less than or equal to 0.1 mg/kg per day (e.g., less than or equal to 0.09, 0.08, 0.07, 0.06, 0.5, 0.04, 0.03, 0.02 or 0.01 mg/kg/day). In some important embodiments, the agent of Formula I is administered in a dose of about 0.005 mg/kg to less than or equal to 1.0 mg/kg per day, or a dose of about 0.005 mg/kg to less than or equal to 0.1 mg/kg per day.

The invention is further based, in part, on the surprising discovery that administration of linear or cyclic Formula I compound and another therapeutic agent (e.g., an anti-cancer antibody or antibody fragment) has unexpected benefit over the administration of either agent alone. In some instances, the effect is additive, and in others it is synergistic.

Thus, in one aspect of the invention, the Formula I compound and the other therapeutic agent are administered as a synergistic combination in an effective amount to treat or reduce the risk of developing a cancer. As used herein, the term “synergistic” describes an effect resulting from the combination of at least two agents which is greater than the effect of each of the individual agents when used alone. When used together either or both agents may be used at lower doses than would be used if either agent was administered alone. In these embodiments, either agent or both may be administered in a “sub-therapeutic” dose for each alone, the combination, however, being therapeutic.

A “sub-therapeutic dose” as used herein refers to a dosage which is less than that dosage which would produce a therapeutic result in the subject if administered in the absence of the other agent. For example, the sub-therapeutic dose of a anti-cancer antibody is one which would not produce the desired therapeutic result in the subject in the absence of the administration of the Formula I compound. Therapeutic doses of anti-cancer antibodies are well known in the field of medicine for the treatment of cancer. These dosages have been extensively described in references such as Remington's Pharmaceutical Sciences, 18th ed., 1990, or the Physician Desktop Reference; as well as many other medical references relied upon by the medical profession as guidance for the treatment of cancer.

For any compound described herein an effective amount can be initially determined from cell culture assays. In particular, the effective amount of a Formula I compound can be determined using in vitro stimulation assays. The stimulation index of immune cells can be used to determine an effective amount of the particular compound for the particular subject, and the dosage can be adjusted upwards or downwards to achieve the desired levels in the subject.

Effective amounts can also be determined in animal studies. For instance, the effective amount of a Formula I compound and an anti-cancer antibody to induce a synergistic response can be assessed using in vivo assays of tumor regression and/or prevention of tumor formation. Relevant animal models include assays in which malignant cells are injected into the animal subjects, usually in a defined site. Generally, a range of Formula I compound doses are administered into the animal along with a range of anti-cancer antibody doses. Inhibition of the growth of a tumor following the injection of the malignant cells is indicative of the ability to reduce the risk of developing a cancer. Inhibition of further growth (or reduction in size) of a pre-existing tumor is indicative of the ability to treat the cancer. Mice which have been modified to have human immune system elements can be used as recipients of human cancer cell lines to determine the effective amount of the synergistic combination.

The agents of Formula I however are administered by injection or in an enterically coated form. If the agent is administered orally, it is provided in an enteric coating. Such enteric coatings are known in the art and a brief description of them follows.

In some embodiments, the agents of Formula I are intended to be released solely in the intestine in order to avoid the aminopeptidases present in the upper GI tract. Release only in the intestine can be achieved using conventional enteric coatings such as pH sensitive coatings which dissolve in the pH environment of the intestine (but not the stomach) or coatings which dissolve over time.

Delivery systems specific for the gastrointestinal tract are roughly divided into three types: the first is a delayed release system designed to release a drug in agreement with, for example, a change in pH; the second is a timed-release system designed to release a drug after a predetermined time; and the third is a microflora enzyme system making use of the abundant enterobacteria in the lower part of the gastrointestinal tract (e.g., in a colonic site-directed release formulation).

An example of a delayed release system is one that uses, for example, an acrylic or cellulosic coating material and dissolves on pH change. Because of ease of preparation, many reports on such “enteric coatings” have been made. In general, an enteric coating is one which passes through the stomach without releasing substantial amounts of drug in the stomach (i.e., less than 10% release, 5% release and even 1% release in the stomach) and sufficiently disintegrating in the intestine tract (by contact with approximately neutral or alkaline intestine juices) to allow the resorption of the active agent through the walls of the intestinal tract.

Various in vitro tests for determining whether or not a coating is classified as an enteric coating have been published in the pharmacopoeia of various countries. A coating which remains intact for at least 2 hours, in contact with artificial gastric juices such as HCl of pH 1 at 36 to 38° C. and thereafter disintegrates within 30 minutes in artificial intestinal juices such as a KH₂PO₄ buffered solution of pH 6.8 is one example. One such well known system is EUDRAGIT material, commercially available and reported on by Behringer, Manchester University, Saale Co., and the like. Enteric coatings are discussed further, below.

A timed release system is represented by Time Erosion System (TES) by Fujisawa Pharmaceutical Co., Ltd. and Pulsincap by R. P. Scherer. According to these systems, the site of drug release is decided by the time of transit of a preparation in the gastrointestinal tract. Since the transit of a preparation in the gastrointestinal tract is largely influenced by the gastric emptying time, some time release systems are also enterically coated.

Systems making use of the enterobacteria can be classified into those utilizing degradation of azoaromatic polymers by an azo reductase produced from enterobacteria as reported by the group of Ohio University (M. Saffran, et al., Science, Vol. 233: 1081 (1986)) and the group of Utah University (J. Kopecek, et al., Pharmaceutical Research, 9(12), 1540-1545 (1992)); and those utilizing degradation of polysaccharides by beta-galactosidase of enterobacteria as reported by the group of Hebrew University (unexamined published Japanese patent application No. 5-50863 based on a PCT application) and the group of Freiberg University (K. H. Bauer et al., Pharmaceutical Research, 10(10), S218 (1993)). In addition, the system using chitosan degradable by chitosanase by Teikoku Seiyaku K. K. (unexamined published Japanese patent application No. 4-217924 and unexamined published Japanese patent application No. 4-225922) is also included.

The enteric coating is typically although not necessarily a polymeric material. Preferred enteric coating materials comprise bioerodible, gradually hydrolyzable and/or gradually water-soluble polymers. The “coating weight,” or relative amount of coating material per capsule, generally dictates the time interval between ingestion and drug release. Any coating should be applied to a sufficient thickness such that the entire coating does not dissolve in the gastrointestinal fluids at pH below about 5, but does dissolve at pH about 5 and above. It is expected that any anionic polymer exhibiting a pH-dependent solubility profile can be used as an enteric coating in the practice of the present invention The selection of the specific enteric coating material will depend on the following properties: resistance to dissolution and disintegration in the stomach; impermeability to gastric fluids and drug/carrier/enzyme while in the stomach; ability to dissolve or disintegrate rapidly at the target intestine site; physical and chemical stability during storage; non-toxicity; ease of application as a coating (substrate friendly); and economical practicality.

Suitable enteric coating materials include, but are not limited to: cellulosic polymers such as cellulose acetate phthalate, cellulose acetate trimellitate, hydroxypropylmethyl cellulose phthalate, hydroxypropyhmethyl cellulose succinate and carboxymethylcellulose sodium; acrylic acid polymers and copolymers, preferably formed from acrylic acid, methacrylic acid, methyl acrylate, ammonio methylacrylate, ethyl acrylate, methyl methacrylate and/or ethyl methacrylate (e.g., those copolymers sold under the tradename EUDRAGIT); vinyl polymers and copolymers such as polyvinyl acetate, polyvinylacetate phthalate, vinylacetate crotonic acid copolymer, and ethylene-vinyl acetate copolymers; and shellac (purified lac). Combinations of different coating materials may also be used. Well known enteric coating material for use herein are those acrylic acid polymers and copolymers available under the tradename EUDRAGIT from Rohm Pharma (Germany). The EUDRAGIT series E, L, S, RL, RS and NE copolymers are available as solubilized in organic solvent, as an aqueous dispersion, or as a dry powder. The EUDRAGIT series RL, NE, and RS copolymers are insoluble in the gastrointestinal tract but are permeable and are used primarily for extended release. The EUDRAGIT series E copolymers dissolve in the stomach. The EUDRAGIT series L, L-30D and S copolymers are insoluble in stomach and dissolve in the intestine, and are thus most preferred herein.

A particular methacrylic copolymer is EUDRAGIT L, particularly L-30D and EUDRAGIT 10055. In EUDRAGIT L-30D, the ratio of free carboxyl groups to ester groups is approximately 1:1. Further, the copolymer is known to be insoluble in gastrointestinal fluids having pH below 5.5, generally 1.5-5.5, i.e., the pH generally present in the fluid of the upper gastrointestinal tract, but readily soluble or partially soluble at pH above 5.5, i.e., the pH generally present in the fluid of lower gastrointestinal tract. Another particular methacrylic acid polymer is EUDRAGIT S, which differs from EUDRAGIT L-30D in that the ratio of free carboxyl groups to ester groups is approximately 1:2. EUDRAGIT S is insoluble at pH below 5.5, but unlike EUDRAGIT L-30D, is poorly soluble in gastrointestinal fluids having a pH in the range of 5.5 to 7.0, such as in the small intestine. This copolymer is soluble at pH 7.0 and above, i.e., the pH generally found in the colon. EUDRAGIT S can be used alone as a coating to provide drug delivery in the large intestine. Alternatively, EUDRAGIT S, being poorly soluble in intestinal fluids below pH 7, can be used in combination with EUDRAGIT L-30D, soluble in intestinal fluids above pH 5.5, in order to provide a delayed release composition which can be formulated to deliver the active agent to various segments of the intestinal tract. The more EUDRAGIT L-30D used, the more proximal release and delivery begins, and the more EUDRAGIT S used, the more distal release and delivery begins. It will be appreciated by those skilled in the art that both EUDRAGIT L-30D and EUDRAGIT S can be replaced with other pharmaceutically acceptable polymers having similar pH solubility characteristics.

The enteric coating provides for controlled release of the active agent, such that drug release can be accomplished at some generally predictable location. The enteric coating also prevents exposure of the therapeutic agent and carrier to the epithelial and mucosal tissue of the buccal cavity, pharynx, esophagus, and stomach, and to the enzymes associated with these tissues. The enteric coating therefore helps to protect the active agent, carrier and a patient's internal tissue from any adverse event prior to drug release at the desired site of delivery. Furthermore, the coated material of the present invention allow optimization of drug absorption, active agent protection, and safety. Multiple enteric coatings targeted to release the active agent at various regions in the gastrointestinal tract would enable even more effective and sustained improved delivery throughout the gastrointestinal tract.

The coating can, and usually does, contain a plasticizer to prevent the formation of pores and cracks that would permit the penetration of the gastric fluids. Suitable plasticizers include, but are not limited to, triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, a coating comprised of an anionic carboxylic acrylic polymer will usually contain approximately 10% to 25% by weight of a plasticizer, particularly dibutyl phthalate, polyethylene glycol, triethyl citrate and triacetin. The coating can also contain other coating excipients such as detackifiers, antifoaming agents, lubricants (e.g., magnesium stearate), and stabilizers (e.g., hydroxypropylcellulose, acids and bases) to solubilize or disperse the coating material, and to improve coating performance and the coated product.

The coating can be applied to particles of the therapeutic agent(s), tablets of the therapeutic agent(s), capsules containing the therapeutic agent(s) and the like, using conventional coating methods and equipment. For example, an enteric coating can be applied to a capsule using a coating pan, an airless spray technique, fluidized bed coating equipment, or the like. Detailed information concerning materials, equipment and processes for preparing coated dosage forms may be found in Pharmaceutical Dosage Forms: Tablets, eds. Lieberman et al. (New York: Marcel Dekker, Inc., 1989), and in Ansel et al., Pharmaceutical Dosage Forms and Drug Delivery Systems, 6th Ed. (Media, Pa.: Williams & Wilkins, 1995). The coating thickness, as noted above, must be sufficient to ensure that the oral dosage form remains intact until the desired site of topical delivery in the lower intestinal tract is reached.

The therapeutic agents may be provided in coated capsules, pills, lozenges and the like. The capsule material may be either hard or soft, and as will be appreciated by those skilled in the art, typically comprises a tasteless, easily administered and water soluble compound such as gelatin, starch or a cellulosic material. The capsules are preferably sealed, such as with gelatin bands or the like. See, for example, Remington: The Science and Practice of Pharmacy, Nineteenth Edition (Easton, Pa.: Mack Publishing Co., 1995), which describes materials and methods for preparing encapsulated pharmaceuticals.

The administration may also be accomplished by non-oral routes that avoid the gastrointestinal tract. These include parenteral routes such as subcutaneous, intravenous, intramuscular, intraperitoneal, intra-tumor or infusion. In these embodiments, the agents of Formula I may be provided in a vial or ampoule preferably with a septum. The agents may be dry thereby requiring reconstitution with an appropriate diluent or carrier.

The other therapeutic agents may be administered by any route available as described below. The particular mode selected will depend, of course, upon the agent selected, the condition being treated, the severity of the condition, whether the treatment is therapeutic or prophylactic, and the dosage required for efficacy. The methods of the invention, generally speaking, may be practiced using any mode of administration that is medically acceptable, meaning any mode that produces effective levels of the active compounds without causing clinically unacceptable adverse effects.

The administration route of the agents of Formula I is not limiting on the administration route of the other therapeutic agents described herein. The Formula I compound may be administered in the same route, and in the same formulation as the other therapeutic agents, or it may be administered in a different route, different formulation, and even on a different schedule.

When using the agent of the invention in subjects in whom the primary site of abnormal proliferation is well delineated and easily accessible, direct administration to the site may be preferred, provided the tumor has not already metastasized. For example, administration by inhalation for lung tumors or by suppositories in the treatment of cervical, ovarian or rectal tumors may be preferred. Likewise, melanoma, for example, may be treated with the agent via topical administration in and around the area of the lesion. In still other embodiments aimed at the treatment of subjects with breast or prostate cancer, the agents may be delivered by injection directly into the tissue with, for example, a biopsy needle and syringe.

Systemic administration may be preferred in some instances such as, for example, if the subject is known to have or is suspected of having metastases. In this way, all tumor sites, whether primary or secondary may receive the agent. Systemic delivery may be accomplished through for example oral administration in an enteric coating, or by injection.

As discussed earlier, the agent may also be delivered to a tumor site during or immediately after a surgical procedure to remove the tumor by lavage into the excision site or by perfusion of the affected tissue with a physiologically acceptable solution containing the agent. Alternatively, the patient may be administered the agent prior to or following the surgical procedure by continuous infusion. In yet other embodiments, a sustained release device, as described below, such as a polymeric implant may be positioned during surgery in the vicinity of the excision site so as to provide a high local concentration of the agent. These latter embodiments may be appropriate to prevent regrowth of the tumor.

The agent of the invention may be administered alone or in combination with the above-described chemotherapies as part of a pharmaceutical composition. Such a pharmaceutical composition may include the agent in combination with any standard physiologically and/or pharmaceutically acceptable carriers which are known in the art. The compositions should be sterile and contain either a therapeutically or prophylactically effective amount of the agent in a unit of weight or volume suitable for administration to a subject. The term “pharmaceutically-acceptable carrier” as used herein means one or more compatible solid or liquid filler, diluents or encapsulating substances which are suitable for administration into a subject of the invention. The term “carrier” denotes an organic or inorganic ingredient, natural or synthetic, with which the active ingredient is combined to facilitate the application. The components of the pharmaceutical compositions also are capable of being co-mingled with the molecules of the present invention, and with each other, in a manner such that there is no interaction which would substantially impair the desired pharmaceutical efficacy. Pharmaceutically-acceptable further means a non-toxic material that is compatible with a biological system such as a cell, cell culture, tissue, or organism. The characteristics of the carrier will depend on the route of administration. Physiologically and pharmaceutically-acceptable carriers include diluents, fillers, salts, buffers, stabilizers, solubilizers, and other materials which are well known in the art.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

Compositions suitable for parenteral administration conveniently comprise a sterile aqueous preparation of the agent, which is preferably isotonic with the blood of the recipient. This aqueous preparation may be formulated according to known methods using suitable dispersing or wetting compounds and suspending compounds. The sterile injectable preparation also may be a sterile injectable solution or suspension in a non-toxic parenterally-acceptable diluent or solvent, for example, as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed including synthetic mono- or di-glycerides. In addition, fatty acids such as oleic acid may be used in the preparation of injectables. Carrier formulations suitable for oral, subcutaneous, intravenous, intramuscular, etc. administrations can be found in Remington's Pharmaceutical Sciences, Mack Publishing Co., Easton, Pa.

Preparations for parenteral administration include sterile aqueous or non-aqueous solutions. suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating compounds, and inert gases and the like. The pharmaceutical compositions may conveniently be presented in unit dosage form and may be prepared by any of the methods well-known in the art of pharmacy.

Compositions suitable for oral administration may be presented as discrete units, such as capsules, tablets, lozenges, each containing a predetermined amount of the agent. Other compositions include suspensions in aqueous liquids or non-aqueous liquids such as a syrup, elixir or an emulsion.

For oral administration, the agents can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers for neutralizing internal acid conditions or may be administered without any carriers.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch. Techniques for preparing aerosol delivery systems are well known to those of skill in the art. Generally, such systems should utilize components which will not significantly impair the biological properties of the therapeutic (see, for example, Sciarra and Cutie, “Aerosols,” in Remington's Pharmaceutical Sciences, 18th edition, 1990, pp 1694-1712; incorporated by reference). Those of skill in the art can readily determine the various parameters and conditions for producing aerosols without resort to undue experimentation.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

The compounds may also be formulated in rectal or vaginal compositions such as suppositories or retention enemas, e.g., containing conventional suppository bases such as cocoa butter or other glycerides.

In some important embodiments, the antigens or antibodies are administered mucosally. In these and other embodiments, the subject may be passively or actively exposed to an antigen. Passive exposure occurs when the subject comes in contact with an antigen, such as an infectious pathogen, by being in an environment in which the pathogen is present, and unbeknownst to the subject. Active exposure on the other hand occurs when the subject is deliberately administered an antigen generally for the purpose of vaccination. Passive exposure to infectious pathogens often occurs at the mucosal surfaces such as the oral, nasal, vaginal, penile, and rectal surfaces. Accordingly, the invention embraces exposure of antigens at these surfaces, prior to, substantially simultaneously with, and/or following administration of compounds of Formula I.

In some embodiments, it is preferred that antigens and antibodies by administered by routes that mimic the routes through which antigens or carcinogens would enter the body of the subject. For example, if the antigen is from a respiratory virus, then in some instances it is preferable to administer the antigen by inhalation. Similarly, if the antigen is from a microbe that is generally transmitted by sexual intercourse, then in some instances it is preferable to administer such antigens or antibodies to a vaginal, penile or rectal surface.

In still other embodiments, the Formula I compounds are administered locally, and optionally the antigens or antibodies are administered locally as well.

In addition to the formulations described previously, the compounds may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer, Science 249:1527-1533, 1990, which is incorporated herein by reference.

In yet other embodiments, the preferred vehicle is a biocompatible microparticle or implant that is suitable for implantation into the mammalian recipient. Exemplary bioerodible implants that are useful in accordance with this method are described in PCT International Application No. PCT/US/03307 (Publication No. WO 95/24929, entitled “Polymeric Gene Delivery System”, claiming priority to U.S. patent application Ser. No. 213,668, filed Mar. 15, 1994). PCT/US/0307 describes a biocompatible, preferably biodegradable polymeric matrix for containing a biological macromolecule. The polymeric matrix may be used to achieve sustained release of the agent in a subject. In accordance with one aspect of the instant invention, the agent described herein may be encapsulated or dispersed within the biocompatible, preferably biodegradable polymeric matrix disclosed in PCT/US/03307. The polymeric matrix preferably is in the form of a microparticle such as a microsphere (wherein the agent is dispersed throughout a solid polymeric matrix) or a microcapsule (wherein the agent is stored in the core of a polymeric shell). Other forms of the polymeric matrix for containing the agent include films, coatings, gels, implants, and stents. The size and composition of the polymeric matrix device is selected to result in favorable release kinetics in the tissue into which the matrix device is implanted. The size of the polymeric matrix devise further is selected according to the method of delivery which is to be used, typically injection into a tissue or administration of a suspension by aerosol into the nasal and/or pulmonary areas. The polymeric matrix composition can be selected to have both favorable degradation rates and also to be formed of a material which is bioadhesive, to further increase the effectiveness of transfer when the devise is administered to a vascular or pulmonary surface. The matrix composition also can be selected not to degrade, but rather, to release by diffusion over an extended period of time.

Both non-biodegradable and biodegradable polymeric matrices can be used to deliver the agents of the invention to the subject. Biodegradable matrices are preferred. Such polymers may be natural or synthetic polymers. Synthetic polymers are preferred. The polymer is selected based on the period of time over which release is desired, generally in the order of a few hours to a year or longer. Typically, release over a period ranging from between a few hours and three to twelve months is most desirable. The polymer optionally is in the form of a hydrogel that can absorb up to about 90% of its weight in water and further, optionally is cross-linked with multi-valent ions or other polymers.

In general, the agents of the invention may be delivered using the bioerodible implant by way of diffusion, or more preferably, by degradation of the polymeric matrix. Exemplary synthetic polymers which can be used to form the biodegradable delivery system include: polyamides, polycarbonates, polyalkylenes, polyalkylene glycols, polyalkylene oxides, polyalkylene terepthalates, polyvinyl alcohols, polyvinyl ethers, polyvinyl esters, poly-vinyl halides, polyvinylpyrrolidone, polyglycolides, polysiloxanes, polyurethanes and co-polymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, poly vinyl chloride, polystyrene and polyvinylpyrrolidone.

Examples of non-biodegradable polymers include ethylene vinyl acetate, poly(meth)acrylic acid, polyamides, copolymers and mixtures thereof.

Examples of biodegradable polymers include synthetic polymers such as polymers of lactic acid and glycolic acid, polyanhydrides, poly(ortho)esters, polyurethanes, poly(butic acid), poly(valeric acid), and poly(lactide-cocaprolactone), and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion. Bioadhesive polymers of particular interest include bioerodible hydrogels described by H. S. Sawhney, C. P. Pathak and J. A. Hubell in Macromolecules, 1993, 26, 581-328, the teachings of which are incorporated herein, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate). Thus, the invention provides a composition of the above-described agents of Formula I for use as a medicament, methods for preparing the medicament and methods for the sustained release of the medicament in vivo.

Other delivery systems can include timed release, delayed release or sustained release delivery systems. Such systems can avoid repeated administrations of the agent of the invention, increasing convenience to the subject and the physician. Many types of release delivery systems are available and known to those of ordinary skill in the art. They include the above-described polymeric systems, as well as polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono-di- and tri-glycerides; hydrogel release systems; silastic systems; peptide based systems; wax coatings; compressed tablets using conventional binders and excipients; partially fused implants; and the like. Specific examples include, but are not limited to: (a) erosional systems in which the agent is contained in a form within a matrix such as those described in U.S. Pat. Nos. 4,452,775, 4,675,189 and 5,736,152 and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation.

Use of a long-term sustained release implant may be particularly suitable for treatment of chronic conditions, such as a the suspected presence of dormant metastases. Long-term release, are used herein, means that the implant is constructed and arranged to delivery therapeutic levels of the active ingredient for at least 30 days, at least 60 days and more preferably for several months. Long-term sustained release implants are well-known to those of ordinary skill in the art and include some of the release systems described above.

According to another aspect of the invention, a kit is provided. The kit is a package which houses a container which contains an agent of the invention and also houses instructions for administering the agent of the invention to a subject having a condition characterized by an abnormal mammalian cell proliferation. The kit may optionally also contain one or more other anti-proliferative compounds or one or more anti-angiogenic compounds for use in combination therapies as described herein.

The kits may comprise in one container an antibody or antibody fragment, preferably formulated and contained for administration by injection, and in another container the agent of Formula I, formulated either for injection or in enterically coated form for oral administration. As another example, the kits may comprise in one container both the agent of Formula I and an antigen, or a cocktail of antigens. Alternatively, the Formula I compounds and the antigens may be provided in the same kit but in different containers, and in different formulations for different administration routes. In some embodiments, it is preferred to provide all the active agents in a powdered form such as a lyophilized form that can be reconstituted prior to administration to a subject. All the kits of the invention can optionally contain instructions for storage, reconstitution (if applicable) and administration.

In some particular embodiments, the agents of Formula I are provided in pharmaceutical compositions and kits intended for administration by injection (e.g., subcutaneous injection) or via an enterically coated form such as a pill, capsule and the like. The kits may comprise the agents of Formula I separate from the pharmaceutically acceptable carrier. That is the agents of Formula I may be provided in a dry form in a vial or ampoule with a septum, and thereby intended for reconstitution with a diluent, acid solution or pharmaceutically acceptable carrier. These carriers are preferably isotonic solutions. In some instances the agents are reconstituted in an acid solution but then neutralized just prior to administration with a diluent having a higher pH. The diluent may be a neutral or basic solution. It is preferred that the agents be reconstituted and/or neutralized shortly prior to administration (e.g., within 3, 2 or 1 hour or within 30 minutes of administration). If the agent is provided in an acid solution then it may be maintained in that form indefinitely. Accordingly, indefinite storage of the agent can be accomplished either in a dry form or in an acid solution. The kits may also include a plurality of containers reflecting the number of administrations to be given to a subject. If the kit contains a first and second container, then a plurality of these would be present. These kits would include instructions for reconstitution, storage, and use.

The invention will be more fully understood by reference to the following Examples.

### EXAMPLES

### Example 1

### : Ile-boroPro has Greater In Vivo Activity When Administered by Subcutaneous Injection Than When Administered Orally.

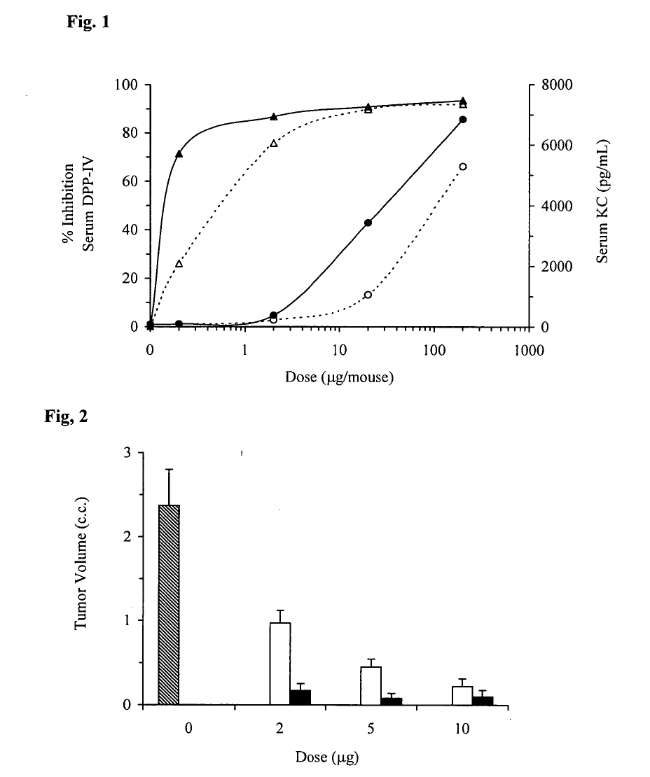

Mice were administered 0.2-, 2.0-, 20-, and 200-μg doses of Ile-boroPro by subcutaneous injection or oral gavage. Two hours later, serum samples were obtained for determination of DPP-IV activity using the fluorogenic substrate Ala-Pro-7-amino-4-trifluoromethyl coumarin and the levels of the chemokine KC by ELISA. The data allowed comparison of dose responses for the inhibition of serum DPP-IV and the increase in levels of KC when Ile-boroPro was administered by the two different routes (FIG. 1). Serum DPP-IV activity served as an indicator of bioavailability because in vitro, DPP-IV cleavage of Ala-Pro-7-amino-4-trifluoromethyl coumarin was inhibited by Ile-boroPro (IC₅₀ values of 0.20.8 nM). KC was chosen as a marker for the cytokine and chemokine response because maximal increases in serum levels have been observed to occur at 2 hours after Ile-boroPro administration, thereby allowing DPP-IV and KC assays of the same serum sample. Comparison of the dose response curves (FIG. 1) for the oral and subcutaneous routes of administration indicate that Ile-boroPro was both more bioavailable and more potent in the induction of a KC response when given by subcutaneous injection.

### Example 2

### Ile-boroPro has a Greater In Vivo Anti-Tumor Effect When Administered by Subcutaneous Injection Than When Administered Orally.

Mice were inoculated subcutaneously with 4×10⁶ WEHI 164 tumor cells and administered 2-, 5- or 10-μg doses of Ile-boroPro twice daily from day 2 to day 9 after tumor inoculation, either by oral gavage or subcutaneous injection. Control mice received saline. Comparison of tumor volumes on day 20 after tumor inoculation indicated that subcutaneous administration was more effective than oral administration in suppressing tumor growth (FIG. 2). For the 2- and 5-μg doses, tumor sizes were significantly smaller (2-μg: P<0.0005; 5-μg: P<0.005) in mice receiving Ile-boroPro subcutaneously compared to orally. The greater efficacy of subcutaneous administration was also reflected in the incidence of tumor rejection in mice receiving treatment with the various doses of Ile-boroPro (Table 2). When administered orally, only the 10-μg dose of Ile-boroPro caused tumor rejection, whereas both the 2- and 5-μg dose caused rejection in 60 and 70 percent, respectively, of the mice treated by subcutaneous injection. It should be noted that tumor rejection was never observed in mice inoculated with WEHI 164 and treated with saline.

TABLE 2
Comparison of WEHI 164 tumor rejection in mice
administered IBP by oral and subcutaneous routes

Incidence of tumor rejection (%)²

| Dose (μg)¹ | Oral IBP | Subcutaneous IBP |
|---|---|---|
| 2 | 0/18 (0) | 6/10 (60) |
| 5 | 0/18 (0) | 7/10 (70) |
| 10 | 11/18 (61) | 7/10 (70) |

¹Each dose was administered twice daily by oral gavage from day 2 to day 19 after tumor inoculation.
²Tumor rejection was recorded on day 20 after tumor inoculation. Oral data are from 2 separate experiments and subcutaneous data are from one experiment.

### Equivalents

It should be understood that the preceding is merely a detailed description of certain embodiments of the invention. These examples, however, are merely intended to illustrate the embodiments of the invention and are not to be construed to limit the scope of the invention. It therefore should be apparent to those skilled in the art that various modifications and equivalents can be made without departing from the spirit and scope of the invention. It is intended to encompass all such modifications within the scope of the appended claim.

All references, patents and patent publications that are recited in this application are incorporated in their entirety herein by reference.

We claim:

1. A method for treating a condition characterized by abnormal mammalian cell proliferation comprising
administering to a subject in need thereof an agent of Formula I in an effective amount to inhibit the condition,
wherein the agent of Formula I is administered by injection or in an enterically coated form.

2. The method of claim 1, wherein the condition is a cancer.

3. The method of claim 1, wherein the condition is a premalignant condition.

4. The method of claim 1, wherein the condition is a benign tumor.

5. The method of claim 1, wherein the abnormal cell proliferation is abnormal angiogenesis.

6. The method of claim 1, further comprising administering to the subject an anti-cancer therapy other than an agent of Formula I.

7. The method of claim 6, wherein the anti-cancer therapy is surgery, radiation or chemotherapy.

8. The method of claim 7, wherein chemotherapy is selected from the group consisting of aldesleukin, asparaginase, bleomycin sulfate, carboplatin, chlorambucil, cisplatin, cladribine, cyclophosphamide, cytarabine, dacarbazine, dactinomycin, daunorubicin hydrochloride, docetaxel, doxorubicin, doxorubicin hydrochloride, epirubicin hydrochloride, etoposide, etoposide phosphate, floxuridine, fludarabine, fluorouracil, gemcitabine, gemcitabine hydrochloride, hydroxyurea, idarubicin hydrochloride, ifosfamide, interferons, interferon-α2a, interferon-α2b, interferon-αn3, interferon-α1b, interleukins, irinotecan, mechlorethamine hydrochloride, melphalan, mercatopurine, methotrexate, methotrexate sodium, mitomycin, mitoxantrone, paclitaxel, pegaspargase, pentostatin, prednisone, profimer sodium, procabazine hydrochloride, taxol, taxotere, teniposide, topotecan hydrochloride, vinblastine sulfate, vincristine sulfate or vinorelbine tartrate.

9. The method of claim 6, wherein the agent of Formula I is administered prior to or after the anti-cancer therapy.

10. The method of claim 6, wherein the agent of Formula I is administered substantially simultaneously with the anti-cancer therapy.

11. The method of claim 7, wherein the agent of Formula I is administered daily and the chemotherapy is administered weekly, biweekly, or every three weeks.

12. The method of claim 1, wherein the agent of Formula I is administered twice a day.

13. A method for treating an infectious disease comprising
administering to a subject in need thereof an agent of Formula I in an effective amount to inhibit the infectious disease,
wherein the agent of Formula I is administered by injection or in an enterically coated form.

14. The method of claim 13, further comprising administering to the subject an anti-microbial agent.

15. The method of claim 14, wherein the anti-microbial agent is an anti-bacterial agent, an anti-viral agent, an anti-fungal agent, an anti-parasitic agent or an anti-mycobacterial agent.

16. The method of claim 15, wherein the anti-microbial agent is an anti-bacterial agent.

17. The method of claim 16, wherein the anti-bacterial agent is an antibiotic.

18. The method of claim 17, wherein the antibiotic is a broad spectrum antibiotic, a narrow spectrum antibiotic, or a limited spectrum antibiotic.

19. The method of claim 16, wherein the anti-bacterial agent is selected from the group consisting of cell wall synthesis inhibitor, cell membrane inhibitor, protein synthesis inhibitor, nucleic acid synthesis or functional inhibitor and competitive inhibitor.

20. The method of claim 16, wherein the anti-bacterial agent is selected from the group consisting of natural penicillins, semi-synthetic penicillins, clavulanic acid, cephalolsporins, bacitracin, ampicillin, carbenicillin, oxacillin, azlocillin, mezlocillin, piperacillin, methicillin, dicloxacillin, nafcillin, cephalothin, cephapirin, cephalexin, cefamandole, cefaclor, cefazolin, cefuroxine, cefoxitin, cefotaxime, cefsulodin, cefetamet, cefixime, ceftriaxone, cefoperazone, ceftazidine, moxalactam, carbapenems, imipenems, monobactems, euztreonam, vancomycin, polymyxin, amphotericin B, nystatin, imidazoles, clotrimazole, miconazole, ketoconazole, itraconazole, fluconazole, rifampins, ethambutol, tetracyclines, chloramphenicol, macrolides, aminoglycosides, streptomycin, kanamycin, tobramycin, amikacin, gentamicin, tetracycline, minocycline, doxycycline, chlortetracycline, erythromycin, roxithromycin, clarithromycin, oleandomycin, azithromycin, chloramphenicol, quinolones, co-trimoxazole, norfloxacin, ciprofloxacin, enoxacin, nalidixic acid, temafloxacin, sulfonamides, gantrisin, and trimethoprim.

21. The method of claim 16, wherein the anti-bacterial agent is selected from the group consisting of acedapsone; acetosulfone sodium; alamecin; alexidine; amdinocillin; amdinocillin pivoxil; amicycline; amifloxacin; amifloxacin mesylate; amikacin; amikacin sulfate; aminosalicylic acid; aminosalicylate sodium; amoxicillin; amphomycin; ampicillin; ampicillin sodium; apalcillin sodium; apramycin; aspartocin; astromicin sulfate; avilamycin; avoparcin; azithromycin; azlocillin; azlocillin sodium; bacampicillin hydrochloride; bacitracin; bacitracin methylene disalicylate; bacitracin zinc; bambermycins; benzoylpas calcium; berythromycin; betamicin sulfate; biapenem; biniramycin; biphenamine hydrochloride; bispyrithione magsulfex; butikacin; butirosin sulfate; capreomycin sulfate; carbadox; carbenicillin disodium; carbenicillin indanyl sodium; carbenicillin phenyl sodium; carbenicillin potassium; carumonam sodium; cefaclor; cefadroxil; cefamandole; cefamandole nafate; cefamandole sodium; cefaparole; cefatrizine; cefazaflur sodium; cefazolin; cefazolin sodium; cefbuperazone; cefdinir; cefepime; cefepime hydrochloride; cefetecol; cefixime; cefinenoxime hydrochloride; cefinetazole; cefinetazole sodium; cefonicid monosodium; cefonicid sodium; cefoperazone sodium; ceforanide; cefotaxime sodium; cefotetan; cefotetan disodium; cefotiam hydrochloride; cefoxitin; cefoxitin sodium; cefpimizole; cefpimizole sodium; cefpiramide; cefpiramide sodium; cefpirome sulfate; cefpodoxime proxetil; cefprozil; cefroxadine; cefsulodin sodium; ceftazidime; ceftibuten; ceftizoxime sodium; ceftriaxone sodium; cefuroxime; cefuroxime axetil; cefuroxime pivoxetil; cefuroxime sodium; cephacetrile sodium; cephalexin; cephalexin hydrochloride; cephaloglycin; cephaloridine; cephalothin sodium; cephapirin sodium; cephradine; cetocycline hydrochloride; cetophenicol; chloramphenicol; chloramphenicol palmitate; chloramphenicol pantothenate complex; chloramphenicol sodium succinate; chlorhexidine phosphanilate; chloroxylenol; chlortetracycline bisulfate; chlortetracycline hydrochloride; cinoxacin; ciprofloxacin; ciprofloxacin hydrochloride; cirolemycin; clarithromycin; clinafloxacin hydrochloride; clindamycin; clindamycin hydrochloride; clindamycin palmitate hydrochloride; clindamycin phosphate; clofazimine; cloxacillin benzathine; cloxacillin sodium; cloxyquin; colistimethate sodium; colistin sulfate; coumermycin; coumermycin sodium; cyclacillin; cycloserine; dalfopristin; dapsone; daptomycin; demeclocycline; demeclocycline hydrochloride; demecycline; denofungin; diaveridine; dicloxacillin; dicloxacillin sodium; dihydrostreptomycin sulfate; dipyrithione; dirithromycin; doxycycline; doxycycline calcium; doxycycline fosfatex; doxycycline hyclate; droxacin sodium; enoxacin; epicillin; epitetracycline hydrochloride; erythromycin; erythromycin acistrate; erythromycin estolate; erythromycin ethylsuccinate; erythromycin gluceptate; erythromycin lactobionate; erythromycin propionate; erythromycin stearate; ethambutol hydrochloride; ethionamide; fleroxacin; floxacillin; fludalanine; flumequine; fosfomycin; fosfomycin tromethamine; fumoxicillin; furazolium chloride; furazolium tartrate; fusidate sodium; fusidic acid; gentamicin sulfate; gloximonam; gramicidin; haloprogin; hetacillin; hetacillin potassium; hexedine; ibafloxacin; imipenem; isoconazole; isepamicin; isoniazid; josamycin; kanamycin sulfate; kitasamycin; levofuraltadone; levopropylcillin potassium; lexithromycin; lincomycin; lincomycin hydrochloride; lomefloxacin; lomefloxacin hydrochloride; lomefloxacin mesylate; loracarbef; mafenide; meclocycline; meclocycline sulfosalicylate; megalomicin potassium phosphate; mequidox; meropenem; methacycline; methacycline hydrochloride; methenamine; methenamine hippurate; methenamine mandelate; methicillin sodium; metioprim; metronidazole hydrochloride; metronidazole phosphate; mezlocillin; mezlocillin sodium; minocycline; minocycline hydrochloride; mirincamycin hydrochloride; monensin; monensin sodium; nafcillin sodium; nalidixate sodium; nalidixic acid; natamycin; nebramycin; neomycin palmitate; neomycin sulfate; neomycin undecylenate; netilmicin sulfate; neutramycin; nifuradene; nifuraldezone; nifuratel; nifuratrone; nifurdazil; nifurimide; nifurpirinol; nifurquinazol; nifurthiazole; nitrocycline; nitrofurantoin; nitromide; norfloxacin; novobiocin sodium; ofloxacin; ormetoprim; oxacillin sodium; oximonam; oximonam sodium; oxolinic acid; oxytetracycline; oxytetracycline calcium; oxytetracycline hydrochloride; paldimycin; parachlorophenol; paulomycin; pefloxacin; pefloxacin mesylate; penamecillin; penicillin g benzathine; penicillin g potassium; penicillin g procaine; penicillin g sodium; penicillin v; penicillin v benzathine; penicillin v hydrabamine; penicillin v potassium; pentizidone sodium; phenyl aminosalicylate; piperacillin sodium; pirbenicillin sodium; piridicillin sodium; pirlimycin hydrochloride; pivampicillin hydrochloride; pivampicillin pamoate; pivampicillin probenate; polymyxin b sulfate; porfiromycin; propikacin; pyrazinamide; pyrithione zinc; quindecamine acetate; quinupristin; racephenicol; ramoplanin; ranimycin; relomycin; repromicin; rifabutin; rifametane; rifamexil; rifamide; rifampin; rifapentine; rifaximin; rolitetracycline; rolitetracycline nitrate; rosaramicin; rosaramicin butyrate; rosaramicin propionate; rosaramicin sodium phosphate; rosaramicin stearate; rosoxacin; roxarsone; roxithromycin; sancycline; sanfetrinem sodium; sarmoxicillin; sarpicillin; scopafungin; sisomicin; sisomicin sulfate; sparfloxacin; spectinomycin hydrochloride; spiramycin; stallimycin hydrochloride; steffimycin; streptomycin sulfate; streptonicozid; sulfabenz; sulfabenzamide; sulfacetamide; sulfacetamide sodium; sulfacytine; sulfadiazine; sulfadiazine sodium; sulfadoxine; sulfalene; sulfamerazine; sulfameter; sulfamethazine; sulfamethizole; sulfamethoxazole; sulfamonomethoxine; sulfamoxole; sulfanilate zinc; sulfanitran; sulfasalazine; sulfasomizole; sulfathiazole; sulfazamet; sulfisoxazole; sulfisoxazole acetyl; sulfisoxazole diolamine; sulfomyxin; sulopenem; sultamicillin; suncillin sodium; talampicillin hydrochloride; teicoplanin; temafloxacin hydrochloride; temocillin; tetracycline; tetracycline hydrochloride; tetracycline phosphate complex; tetroxoprim; thiamphenicol; thiphencillin potassium; ticarcillin cresyl sodium; ticarcillin disodium; ticarcillin monosodium; ticlatone; tiodonium chloride; tobramycin; tobramycin sulfate; tosufloxacin; trimethoprim; trimethoprim sulfate; trisulfapyrimidines; troleandomycin; trospectomycin sulfate; tyrothricin; vancomycin; vancomycin hydrochloride; virginiamycin; and zorbamycin.

22. The method of claim 15, wherein the anti-microbial agent is an anti-viral agent.

23. The method of claim 22, wherein the anti-viral agent is selected from the group consisting of immunoglobulin, amantadine, interferon, nucleoside analogue, nonnucleoside analogue, biflavanoid and protease inhibitor.

24. The method of claim 23, wherein the protease inhibitor is indinavir, saquinavir, ritonavir, and nelfinavir.

25. The method of claim 23, wherein the biflavanoid is robustaflavone, amentoflavone, or a derivative or salt thereof.

26. The method of claim 22, wherein the antiviral agent is selected from the group consisting of AZT, ddC, ddI, D4T, 3TC, acemannan; acyclovir; acyclovir sodium; adefovir; alovudine; alvircept sudotox; amantadine hydrochloride; aranotin; arildone; atevirdine mesylate; pyridine; cidofovir; cipamfylline; cytarabine hydrochloride; delavirdine mesylate; desciclovir; didanosine; disoxaril; edoxudine; enviradene; enviroxime; famciclovir; famotine hydrochloride; fiacitabine; fialuridine; fluorinated nucleosides; fosarilate; foscarnet sodium; fosfonet sodium; ganciclovir; ganciclovir sodium; idoxuridine; kethoxal; lamivudine; lobucavir; memotine hydrochloride; methisazone; nevirapine; penciclovir; pirodavir; ribavirin; rimantadine hydrochloride; saquinavir mesylate; somantadine hydrochloride; sorivudine; statolon; stavudine; tilorone hydrochloride; trifluridine; valacyclovir hydrochloride; vidarabine; vidarabine phosphate; vidarabine sodium phosphate; viroxime; zalcitabine; zidovudine; and zinviroxime.

27. The method of claim 23, wherein the non-nucleoside analogue is selected from the group consisting of delavirdine, nevirapine, efavirenz, alpha-interferon, recombinant CD4, amantadine, rimantadine, ribavirin and vidarabine.

28. The method of claim 15, wherein the anti-microbial agent is an anti-fungal agent.

29. The method of claim 28, wherein the anti-fungal agent is selected from the group consisting of imidazole, FK 463, amphotericin B, BAY 38-9502, MK 991, pradimicin, UK 292, butenafine, chitinase and 501 cream.

30. The method of claim 28, wherein the anti-fungal agent is selected from the group consisting of acrisorcin; ambruticin; amorolfine, amphotericin b; azaconazole; azaserine; basifungin; bifonazole; biphenamine hydrochloride; bispyrithione magsulfex; butoconazole nitrate; calcium undecylenate; candicidin; carbol-fuchsin; chlordantoin; ciclopirox; ciclopirox olamine; cilofungin; cisconazole; clotrimazole; cuprimyxin; denofungin; dipyrithione; doconazole; econazole; econazole nitrate; enilconazole; ethonam nitrate; fenticonazole nitrate; filipin; fluconazole; flucytosine; fungimycin; griseofulvin; hamycin; isoconazole; itraconazole; kalafungin; ketoconazole; lomofungin; lydimycin; mepartricin; miconazole; miconazole nitrate; monensin; monensin sodium; naftifine hydrochloride; neomycin undecylenate; nifuratel; nifurmerone; nitralamine hydrochloride; nystatin; octanoic acid; orconazole nitrate; oxiconazole nitrate; oxifungin hydrochloride; parconazole hydrochloride; partricin; potassium iodide; proclonol; pyrithione zinc; pyrrolnitrin; rutamycin; sanguinarium chloride; saperconazole; scopafungin; selenium sulfide; sinefungin; sulconazole nitrate; terbinafine; terconazole; thiram; ticlatone; tioconazole; tolciclate; tolindate; tolnaftate; triacetin; triafungin; undecylenic acid; viridofulvin; zinc undecylenate; and zinoconazole hydrochloride.

31. The method of claim 28, wherein the anti-microbial agent is an anti-parasitic agent.

32. The method of claim 15, wherein the anti-parasitic agent is selected from the group consisting of albendazole, amphotericin B, benznidazole, bithionol, chloroquine HCl, chloroquine phosphate, clindamycin, dehydroemetine, diethylcarbamazine, diloxanide furoate, eflornithine, furazolidaone, glucocorticoids, halofantrine, iodoquinol, ivermectin, mebendazole, mefloquine, meglumine antimoniate, melarsoprol, metrifonate, metronidazole, niclosamide, nifurtimox, oxamniquine, paromomycin, pentamidine isethionate, piperazine, praziquantel, primaquine phosphate, proguanil, pyrantel pamoate, pyrimethanmine-sulfonamides, pyrimethanmine-sulfadoxine, quinacrine HCl, quinine sulfate, quinidine gluconate, spiramycin, stibogluconate sodium (sodium antimony gluconate), suramin, tetracycline, doxycycline, thiabendazole, tinidazole, trimethroprim-sulfamethoxazole, and tryparsamide.

33. The method of claim 15, wherein the anti-microbial agent is an anti-mycobacterial agent.

34. The method of claim 33, wherein the anti-mycobacterial agent is an anti-tuberculosis agent.

35. The method of claim 34, wherein the anti-tuberculosis agent is isoniazid, rifampin, rifabutin, rifapentine, pyrazinamide, ethambutol, (+)calanolide A, (−)-calanolide A, (−)-soulattrolide, (−)-costatolide or (−)-7,8-dihydrosoulattrolide

36. The method of claim 33, wherein the anti-mycobacterial agent is streptomycin, dapsone, clarithromycin, ciprofloxacin, clofazamine, azithromycin, ethionamide, amikacin or resorcinomycin A.

37. The method of claim 14, wherein the agent of Formula I is administered prior to or after the anti-microbial agent.

38. The method of claim 14, wherein the agent of Formula I is administered substantially simultaneously with the anti-microbial agent.

39. The method of claim 1 or 13, wherein the agent of Formula I is provided as a pharmaceutical preparation prepared within thirty minutes of administration.

40. A pharmaceutical preparation comprising
an agent of Formula I in a dosage of about 0.005 mg/kg to less than 1.0 mg/kg per day, and
a pharmaceutically acceptable carrier,
wherein the preparation is formulated for injection or in an enterically coated form.

41. The pharmaceutical preparation of claim 40, wherein the preparation is provided in a vial or ampoule with a septum.

42. A kit comprising
a housing and
the pharmaceutical preparation of claim 40.

43. The kit of claim 42, further comprising instructions for use.

44. A pharmaceutical preparation comprising
an agent of Formula I in a dosage of less than 1.0 mg/kg per day,
wherein the preparation is provided in a vial or ampoule with a septum.

45. The pharmaceutical preparation of claim 40 or 44, wherein the dosage is about 0.005 to less than or equal to 0.1 mg/kg per day.

46. The pharmaceutical preparation of claim 40 or 44, wherein the preparation is sterile and pyrogen-free.

47. The pharmaceutical preparation of claim 44, further comprising a pharmaceutically acceptable carrier.

48. The pharmaceutical preparation of claim 40 or 47, wherein the pharmaceutically acceptable carrier comprises a solubilizer, an anti-bacterial preservative, an anti-oxidant or a pharmaceutical adjunct.

49. The pharmaceutical preparation of claim 48, wherein the anti-oxidant is sodium bisulfite.

50. The pharmaceutical preparation of claim 40 or 44, wherein the preparation comprises distilled water or reverse-osmosis water.

51. The pharmaceutical preparation of claim 50, wherein the anti-bacterial preservative is phenylmercuric nitrate, thimerosal, benzetheonium chloride, benzalkonium chloride, phenol, cresol or chlorobutanol.

52. The pharmaceutical preparation of claim 40 or 47, wherein the pharmaceutically acceptable carrier has a pH of less than 5.

53. The pharmaceutical preparation of claim 40 or 47, wherein the pharmaceutically acceptable carrier has a pH of between 2.0 and 5.0.

54. The pharmaceutical preparation of claim 40 or 47, wherein the pharmaceutically acceptable carrier has a pH of between 3.0 and 5.0.

55. The pharmaceutical preparation of claim 40 or 47, wherein the pharmaceutically acceptable carrier has a pH of between 3.0 and 4.5.

56. The pharmaceutical preparation of claim 40 or 47, wherein the pharmaceutically acceptable carrier has a pH of between 3.0 and 4.25.

57. The pharmaceutical preparation of claim 40 or 47, wherein the pharmaceutically acceptable carrier has a pH of between 3.0 and 4.0.

58. The pharmaceutical preparation of claim 40 or 47, wherein the pharmaceutically acceptable carrier has a pH of between 3.0 and 3.5.

59. A method of producing the pharmaceutical preparation of claim 44, comprising combining the agent of Formula I with a pharmaceutically acceptable carrier.

60. The method of claim 59, wherein the combining occurs within 30 minutes of administration to a subject.

61. A kit comprising
a housing that comprises
an agent of Formula I in a first container, and
a pharmaceutically acceptable carrier in a second container,
wherein the agent of Formula I is present in a dried form.

62. The kit of claim 61, wherein the agent and carrier are sterile and pyrogen-free.

63. The kit of claim 61, wherein the pharmaceutically acceptable carrier comprises a solubilizer, an anti-bacterial preservative, an anti-oxidant or a pharmaceutical adjunct.

64. The kit of claim 63, wherein the anti-oxidant is sodium bisulfite.

65. The kit of claim 61, wherein the carrier comprises distilled water or reverse-osmosis water.

66. The kit of claim 63, wherein the anti-bacterial preservative is phenylmercuric nitrate, thimerosal, benzetheonium chloride, benzalkonium chloride, phenol, cresol or chlorobutanol.

67. The kit of claim 61, wherein the pharmaceutically acceptable carrier has a pH of less than 5.

68. The kit of claim 61, wherein the pharmaceutically acceptable carrier has a pH of between 2.0 and 5.0.

69. The kit of claim 61, wherein the pharmaceutically acceptable carrier has a pH of between 3.0 and 5.0.

70. The kit of claim 61, wherein the pharmaceutically acceptable carrier has a pH of between 3.0 and 4.5.

71. The kit of claim 61, wherein the pharmaceutically acceptable carrier has a pH of between 3.0 and 4.25.

72. The kit of claim 61, wherein the pharmaceutically acceptable carrier has a pH of between 3.0 and 4.0.

73. The kit of claim 61, wherein the pharmaceutically acceptable carrier has a pH of between 3.0 and 3.5.

74. The kit of claim 61, wherein the kit comprises a plurality of first and second containers corresponding to a number of administrations to a subject.

75. The kit of claim 61, wherein the first container is a vial or ampoule with a septum.

76. The kit of claim 61 or 75, wherein the second container is a vial or ampoule with a septum.

77. A kit comprising
a housing that comprises
an agent of Formula I dissolved in an acid solution in a first container, and
a neutral or basic isotonic diluent in a second container.

78. The kit of claim 61 or 77, further comprising instructions for administering the agent to a subject in need thereof.

79. The kit of claim 77, wherein the agent, solution and diluent are sterile and pyrogen-free.

80. The kit of claim 77, wherein the acid solution has a pH of less than 5.

81. The kit of claim 77, wherein the acid solution has a pH of between 2.0 and 5.0.

82. The kit of claim 77, wherein the acid solution has a pH of between 3.0 and 5.0.

83. The kit of claim 77, wherein the acid solution has a pH of between 3.0 and 4.5.

84. The kit of claim 77, wherein the acid solution has a pH of between 3.0 and 4.25.

85. The kit of claim 77, wherein the acid solution has a pH of between 3.0 and 4.0.

86. The kit of claim 77, wherein the acid solution has a pH of between 3.0 and 3.5.

87. The kit of claim 77, wherein the diluent has a pH greater than 5.

88. The kit of claim 77, wherein the diluent has a pH of between 5.0 and 8.0.

89. The kit of claim 77, wherein the diluent has a pH of between 5.0 and 7.5.

90. The kit of claim 77, wherein the diluent has a pH of between 5.0 and 7.0.

91. The kit of claim 77, wherein the diluent has a pH of between 5.0 and 6.5.

92. The kit of claim 77, wherein the diluent has a pH of between 5.0 and 6.0.

93. The kit of claim 77, wherein the diluent has a pH of between 5.0 and 5.5.

94. A kit comprising an agent of Formula I in a first container, and instructions for diluting the agent in a neutral or acidic injectable diluent.

95. The kit of claim 94, further comprising a housing comprising the first container and the instructions.

96. The kit of claim 61, 77 or 94, wherein the agent is formulated in a dosage of about 0.005 mg/kg to less than 1.0 mg/kg per day.

97. The kit of claim 61, 77 or 94, wherein the agent is formulated in a dosage of about 0.005 mg/kg to less than or equal to 0.1 mg/kg per day.

98. The kit of claim 94, wherein the agent is sterile and pyrogen-free.

99. The kit of claim 77 or 94, wherein the diluent comprises a solubilizer, an anti-bacterial preservative, an anti-oxidant or a pharmaceutical adjunct.

100. The kit of claim 99, wherein the anti-oxidant is sodium bisulfite.

101. The kit of claim 77 or 94, wherein the diluent comprises distilled water or reverse-osmosis water.

102. The kit of claim 99, wherein the anti-bacterial preservative is phenylmercuric nitrate, thimerosal, benzetheonium chloride, benzalkonium chloride, phenol, cresol or chlorobutanol.

103. The kit of claim 94, wherein the diluent has a pH of less than 7.

104. The kit of claim 94, wherein the diluent has a pH of between 2.0 and 7.0.

105. The kit of claim 94, wherein the diluent has a pH of between 3.0 and 6.0.

106. The kit of claim 94, wherein the diluent has a pH of between 3.0 and 5.0.

107. The kit of claim 94, wherein the diluent has a pH of between 3.0 and 4.25.

108. The kit of claim 94, wherein the diluent has a pH of between 3.0 and 4.0.

109. The kit of claim 94, wherein the diluent has a pH of between 3.0 and 3.5.

110. The kit of claim 61, 77 or 94, wherein the kit comprises a plurality of first containers corresponding to a number of administrations to a subject.

111. The kit of claim 94, wherein the first container is a vial or ampoule with a septum.

112. A method for stimulating an immune response in a subject comprising administering to a subject in need of immune stimulation an agent of Formula I, and an antibody or antibody fragment, in an amount effective to stimulate an immune response,
wherein the agent of Formula I is administered by injection or in an enterically coated form.

113. The method of claim 112, wherein the immune response is antibody dependent cell-mediated cytoxicity.

114. The method of claim 112, wherein the antibody or antibody fragment is an antibody.

115. The method of claim 112, wherein the antibody or antibody fragment is selected from the group consisting of trastuzumab, alemtuzumab (B cell chronic lymphocytic leukemia), gemtuzumab ozogamicin (CD33+ acute myeloid leukemia), hP67.6 (CD33+ acute myeloid leukemia), infliximab (inflammatory bowel disease and rheumatoid arthritis), etanercept (rheumatoid arthritis), rituximab, tositumomab, MDX-210, oregovomab, anti-EGF receptor mAb, MDX-447, anti-tissue factor protein (TF), (Sunol); ior-c5, c5, edrecolomab, ibritumomab tiuxetan, anti-idiotypic mAb mimic of ganglioside GD3 epitope, anti-HLA-Dr10 mAb, anti-CD33 humanized mAb, anti-CD52 humAb, anti-CD1 mAb (ior t6), MDX-22, celogovab, anti-17-IA mAb, bevacizumab, daclizumab, anti-TAG-72 (MDX-220), anti-idiotypic mAb mimic of high molecular weight proteoglycan (I-Mel-1), anti-idiotypic mAb mimic of high molecular weight proteoglycan (I-Mel-2), anti-CEA Ab, hmAbH11, anti-DNA or DNA-associated proteins (histones) mAb, Gliomab-H mAb, GNI-250 mAb, anti-CD22, CMA 676), anti-idiotypic human mAb to GD2 ganglioside, ior egf/r3, anti-ior c2 glycoprotein mAb, ior c5, anti-FLK-2/FLT-3 mAb, anti-GD-2 bispecific mAb, antinuclear autoantibodies, anti-HLA-DR Ab, anti-CEA mAb, palivizumab, bevacizumab, alemtuzumab, BLyS-mAb, anti-VEGF2, anti-Trail receptor; B3 mAb, mAb BR96, breast cancer; and Abx-Cbl mAb.

116. The method of claim 112, wherein the antibody or antibody fragment is an anti-HER2 antibody.

117. The method of claim 116, wherein the anti-HER2 antibody is trastuzumab.

118. The method of claim 112, wherein the antibody or antibody fragment is an anti-CD20 antibody.

119. The method of claim 118, wherein the anti-CD20 antibody is rituximab.

120. The method of claim 112, wherein the antibody or antibody fragment is administered in a sub-therapeutic dose.

121. The method of claim 112, wherein the agent of Formula I is administered in a route of administration different from that of the antibody or antibody fragment.

122. The method of claim 112, wherein the agent of Formula I is administered prior to the antibody or antibody fragment.

123. The method of claim 122, wherein the agent of Formula I is administered 30 minutes to 8 hours prior to the antibody or antibody fragment.

124. The method of claim 122, wherein the agent of Formula I is administered 1 to 7 days prior to the antibody or antibody fragment.

125. The method of claim 112, wherein the agent of Formula I is administered substantially simultaneously with the antibody or antibody fragment.

126. The method of claim 112, wherein the agent of Formula I is administered after the antibody or antibody fragment.

127. The method of claim 126, wherein the agent of Formula I is administered 30 minutes to 8 hours after the antibody or antibody fragment.

128. The method of claim 126, wherein the agent of Formula I is administered 1 to 7 days after the antibody or antibody fragment.

129. A method for stimulating an immune response in a subject comprising
administering to a subject in need of immune stimulation an agent of Formula I, and an antigen, in an amount effective to stimulate an antigen-specific immune response,
wherein the agent of Formula I is administered by injection or in an enterically coated form.

130. The method of claim 112 or 129, wherein the subject in need of immune stimulation is a subject having or at risk of developing cancer.

131. The method of claim 130, wherein the cancer is selected from the group consisting of basal cell carcinoma, biliary tract cancer; bladder cancer; bone cancer; brain cancer; breast cancer; cervical cancer; choriocarcinoma; CNS cancer; colon and rectum cancer; connective tissue cancer; cancer of the digestive system; endometrial cancer; esophageal cancer; eye cancer; cancer of the head and neck; gastric cancer; intra-epithelial neoplasm; kidney cancer; larynx cancer; leukemia; acute myeloid leukemia, acute lymphoid leukemia, chronic myeloid leukemia, chronic lymphoid leukemia, liver cancer; small cell lung cancer; non-small cell lung cancer; lymphoma, Hodgkin's lymphoma; Non-Hodgkin's lymphoma; melanoma; myeloma; neuroblastoma; oral cavity cancer; ovarian cancer; pancreatic cancer; prostate cancer; retinoblastoma; rhabdomyosarcoma; rectal cancer; renal cancer; cancer of the respiratory system; sarcoma; skin cancer; stomach cancer; testicular cancer; thyroid cancer; uterine cancer; and cancer of the urinary system.

132. The method of claim 170, wherein the cancer is a metastasis.

133. The method of claim 112 or 129, wherein the subject in need of immune stimulation is a subject having or at risk of developing an infectious disease.

134. The method of claim 133, wherein the infectious disease is selected from the group consisting of a bacterial infection, a mycobacterial infection, a viral infection, a fungal infection and a parasitic infection.

135. The method of claim 134, wherein the bacterial infection is selected from the group consisting of an E. coli infection, a Staphylococcal infection, a Streptococcal infection, a Pseudomonas infection, Clostridium difficile infection, Legionella infection, Pneumococcus infection, Haemophilus infection, Klebsiella infection, Enterobacter infection, Citrobacter infection, Neisseria infection, Shigella infection, Salmonella infection, Listeria infection, Pasteurella infection, Streptobacillus infection, Spirillum infection, Treponema infection, Actinomyces infection, Borrelia infection, Corynebacterium infection, Nocardia infection, Gardnerella infection, Campylobacter infection, Spirochaeta infection, Proteus infection, Bacteriodes infection, H. pylori infection, and anthrax infection.

136. The method of claim 134, wherein the mycobacterial infection is selected from the group consisting of tuberculosis and leprosy.

137. The method of claim 134, wherein the viral infection is selected from the group consisting of an HIV infection, a Herpes simplex virus 1 infection, a Herpes simplex virus 2 infection, cytomegalovirus infection, hepatitis A virus infection, hepatitis B virus infection, hepatitis C virus infection, human papilloma virus infection, Epstein Barr virus infection, rotavirus infection, adenovirus infection, influenza A virus infection, respiratory syncytial virus infection, varicella-zoster virus infections, small pox infection, monkey pox infection and SARS infection.

138. The method of claim 134, wherein the fungal infection is selected from the group consisting of candidiasis, ringworm, histoplasmosis, blastomycosis, paracoccidioidomycosis, crytococcosis, aspergillosis, chromomycosis, mycetoma infections, pseudallescheriasis, and tinea versicolor infection.

139. The method of claim 134, wherein the parasite infection is selected from the group consisting of amebiasis, Trypanosoma cruzi infection, Fascioliasis, Leishmaniasis, Plasmodium infections, Onchocerciasis, Paragonimiasis, Trypanosoma brucei infection, Pneumocystis infection, Trichomonas vaginal is infection, Taenia infection, Hymenolepsis infection, Echinococcus infections, Schistosomiasis, neurocysticercosis, Necator americanus infection, and Trichuris trichuria infection.

140. The method of claim 112 or 129, wherein the agent of Formula I is administered in a route of administration different from that of the antigen.

141. The method of claim 129, further comprising administering an adjuvant to the subject.

142. The method of claim 141, wherein the adjuvant is selected from the group consisting of alum, cholera toxin, CpG immunostimulatory nucleic acids, MPL, MPD, and QS-21.

143. The method of claim 129, wherein the antigen is targeted to a tissue or a cell.

144. The method of claim 129, wherein the antigen is a cancer antigen.

145. The method of claim 144, further comprising treating the subject with a therapy selected from the group consisting of surgery, radiation and chemotherapy.

146. The method of claim 145, wherein the agent of Formula I and the antigen are administered prior to treating the subject with a therapy selected from the group consisting of surgery, radiation and chemotherapy.

147. The method of claim 145, wherein the agent of Formula I and the antigen are administered after treating the subject with a therapy selected from the group consisting of surgery, radiation and chemotherapy.

148. The method of claim 145, wherein the agent of Formula I and the antigen are administered prior to and after treating the subject with a therapy selected from the group consisting of surgery, radiation and chemotherapy.

149. The method of claim 144, wherein the agent of Formula I is administered to the subject prior to the antigen.

150. The method of claim 149, wherein the agent of Formula I is administered to the subject 30 minutes to 8 hours prior to administration of the antigen.

151. The method of claim 144, wherein the agent of Formula I is administered to the subject 1 to 7 days prior to administration of the antigen.

152. The method of claim 144, wherein the agent of Formula I is administered to the subject after administration of the antigen.

153. The method of claim 152, wherein the agent of Formula I is administered to the subject 30 minutes to 8 hours after administration of the antigen.

154. The method of claim 152, wherein the agent of Formula I is administered to the subject 1 to 7 days after administration of the antigen.

155. The method of claim 129, wherein the immune response is an antigen specific immune response.

156. The method of claim 129, wherein the immune response is an innate immune response or an adaptive immune response.

157. The method of claim 129, wherein the antigen is a microbial antigen.

158. The method of claim 157, wherein the microbial antigen is selected from the group consisting of a bacterial antigen, a mycobacterial antigen, a viral antigen, a fungal antigen, and a parasitic antigen.

159. The method of claim 158, wherein the bacterial antigen is derived from a bacterial species selected from the group consisting of E. coli, Staphylococcal, Streptococcal, Pseudomonas, Clostridium difficile, Legionella, Pneumococcus, Haemophilus, Klebsiella, Enterobacter, Citrobacter, Neisseria, Shigella, Salmonella, Listeria, Pasteurella, Streptobacillus, Spirillum, Treponema, Actinomyces, Borrelia, Corynebacterium, Nocardia, Gardnerella, Campylobacter, Spirochaeta, Proteus, Bacteriodes, H. pylori, and anthrax.

160. The method of claim 158, wherein the viral antigen is derived from a viral species selected from the group consisting of HIV, Herpes simplex virus 1, Herpes simplex virus 2, cytomegalovirus, hepatitis A virus, hepatitis B virus, hepatitis C virus, human papilloma virus, Epstein Barr virus, rotavirus, adenovirus, influenza A virus, respiratory syncytial virus, varicella-zoster virus, small pox, monkey pox and SARS.

161. The method of claim 158, wherein the fungal antigen is derived from a fungal species that causes an infection selected from the group consisting of candidiasis, ringworm, histoplasmosis, blastomycosis, paracoccidioidomycosis, crytococcosis, aspergillosis, chromomycosis, mycetoma infections, pseudallescheriasis, and tinea versicolor infection.

162. The method of claim 158, wherein the parasitic antigen is derived from a parasite species selected from the group consisting of amebiasis, Trypanosoma cruzi, Fascioliasis, Leishmaniasis, Plasmodium, Onchocerciasis, Paragonimiasis, Trypanosoma brucei, Pneumocystis, Trichomonas vaginalis, Taenia, Hymenolepsis, Echinococcus, Schistosomiasis, neurocysticercosis, Necator americanus, and Trichuris trichuria.

163. The method of claim 158, wherein the mycobacterial antigen is derived from a mycobacterial species selected from the group consisting of M. tuberculosis and M. leprae.

164. A method of preventing an infectious disease in a subject at risk of developing an infectious disease comprising
identifying a subject at risk of developing an infectious disease, and
administering an agent of Formula I to the subject in an amount effective to induce IL-1,
wherein the agent of Formula I is administered by injection or in an enterically coated form.

165. The method of claim 164, further comprising administering to the subject a microbial antigen.

166. The method of claim 165, wherein the microbial antigen is selected from the group consisting of a bacterial antigen, a mycobacterial antigen, a viral antigen, a fungal antigen, and a parasitic antigen.

167. The method of claim 166, wherein the bacterial antigen is derived from a bacterial species selected from the group consisting of E. coli, Staphylococcal, Streptococcal, Pseudomonas, Clostridium difficile, Legionella, Pneumococcus, Haemophilus, Klebsiella, Enterobacter, Citrobacter, Neisseria, Shigella, Salmonella, Listeria, Pasteurella, Streptobacillus, Spirillum, Treponema, Actinomyces, Borrelia, Corynebacterium, Nocardia, Gardnerella, Campylobacter, Spirochaeta, Proteus, Bacteriodes, H. pylori, and anthrax.

168. The method of claim 166, wherein the viral antigen is derived from a viral species selected from the group consisting of HIV, Herpes simplex virus 1, Herpes simplex virus 2, cytomegalovirus, hepatitis A virus, hepatitis B virus, hepatitis C virus, human papilloma virus, Epstein Barr virus, rotavirus, adenovirus, influenza A virus, respiratory syncytial virus, varicella-zoster virus, small pox, monkey pox and SARS.

169. The method of claim 166, wherein the fungal antigen is derived from a fungal species that causes an infection selected from the group consisting of candidiasis, ringworm, histoplasmosis, blastomycosis, paracoccidioidomycosis, crytococcosis, aspergillosis, chromomycosis, mycetoma infections, pseudallescheriasis, and tinea versicolor infection.

170. The method of claim 166, wherein the parasitic antigen is derived from a parasite species selected from the group consisting of amebiasis, Trypanosoma cruzi, Fascioliasis, Leishmaniasis, Plasmodium, Onchocerciasis, Paragonimiasis, Trypanosoma brucei, Pneumocystis, Trichomonas vaginalis, Taenia, Hymenolepsis, Echinococcus, Schistosomiasis, neurocysticercosis, Necator americanus, and Trichuris trichuria.

171. The method of claim 166, wherein the mycobacterial antigen is derived from a mycobacterial species selected from the group consisting of M. tuberculosis and M. leprae.

172. A method for stimulating an immune response in a subject having or at risk of having cancer comprising
administering to a subject in need of immune stimulation an agent of Formula I, and an antigen, in an amount effective to stimulate an antigen-specific immune response,
wherein the agent of Formula I is administered by injection or in an enterically coated form.

173. The method of claim 172, wherein the subject is a subject having cancer.

174. The method of claim 172, wherein the subject has or is at risk of developing an infectious disease.

175. The method of claim 172, wherein the agent of Formula I is administered in a route of administration different from that of the antigen.

176. The method of claim 172, further comprising administering an adjuvant to the subject.

177. The method of claim 176, wherein the adjuvant is selected from the group consisting of alum, cholera toxin, CpG immunostimulatory nucleic acids, MPL, MPD, and QS-21.

178. The method of claim 172, wherein the antigen is a cancer antigen.

179. The method of claim 172, further comprising treating the subject with a therapy selected from the group consisting of surgery, radiation and chemotherapy.

180. The method of claim 179, wherein the agent of Formula I and the antigen are administered prior to treating the subject with a therapy selected from the group consisting of surgery, radiation and chemotherapy.

181. The method of claim 179, wherein the agent of Formula I and the antigen are administered after treating the subject with a therapy selected from the group consisting of surgery, radiation and chemotherapy.

182. The method of claim 179, wherein the agent of Formula I and the antigen are administered before and after treating the subject with a therapy selected from the group consisting of surgery, radiation and chemotherapy.

183. The method of claim 172, wherein the subject has not undergone an anti-cancer therapy selected from the group consisting of surgery, radiation and chemotherapy.

184. The method of claim 172, wherein the agent of Formula I is administered to the subject before the antigen.

185. The method of claim 184, wherein the agent of Formula I is administered to the subject 30 minutes to 8 hours before administration of the antigen.

186. The method of claim 184, wherein the agent of Formula I is administered to the subject 1 to 7 days before administration of the antigen.

187. The method of claim 172, wherein the agent of Formula I is administered to the subject after the antigen.

188. The method of claim 187, wherein the agent of Formula I is administered to the subject 30 minutes to 8 hours after administration of the antigen.

189. The method of claim 187, wherein the agent of Formula I is administered to the subject 1 to 7 days after administration of the antigen.

190. The method of claim 172, wherein the immune response is an antigen specific immune response.

191. The method of claim 172, wherein the immune response is an innate immune response or an adaptive immune response.

192. A method for stimulating an immune response in a non-immunocompromised subject comprising
administering to a subject in need thereof an agent of Formula I, in an amount effective to induce IL-1,
wherein the agent of Formula I is administered by injection or in an enterically coated form.

193. The method of claim 192, wherein the subject is a subject having or at risk of developing cancer.

194. The method of claim 192, further comprising administering to the subject an antibody or antibody fragment.

195. The method of claim 192, wherein the subject is elderly.

196. The method of claim 192, wherein the subject is at risk of developing influenza.

197. The method of claim 192, wherein the subject is at risk of angina.

198. A method for stimulating an immune response in an immunocompromised subject comprising
administering to a subject in need thereof an agent of Formula I, in an amount effective to induce IL-1,
wherein the agent of Formula I is administered by injection or in an enterically coated form.

199. The method of claim 198, wherein the immunocompromised subject is genetically immunocompromised.

200. The method of claim 199, wherein the subject has a genetic deficiency selected from the group consisting of SCID, agammaglobulinemia, and CDG.

201. The method of claim 198, wherein the subject has an immunoglobulin deficiency that is common variable immunodeficiency.

202. The method of claim 198, wherein the subject is a subject having or at risk of developing cancer.

203. The method of claim 198, further comprising administering to the subject an antibody or antibody fragment.

204. The method of claim 194 or 203, wherein the antibody or antibody fragment is selected from the group consisting of trastuzumab, alemtuzumab (B cell chronic lymphocytic leukemia), gemtuzumab ozogamicin (CD33+ acute myeloid leukemia), hP67.6 (CD33+ acute myeloid leukemia), infliximab (inflammatory bowel disease and rheumatoid arthritis), etanercept (rheumatoid arthritis), rituximab, tositumomab, MDX-210, oregovomab, anti-EGF receptor mAb, MDX-447, anti-tissue factor protein (TF), (Sunol); ior-c5, c5, edrecolomab, ibritumomab tiuxetan, anti-idiotypic mAb mimic of ganglioside GD3 epitope, anti-HLA-Dr10 mAb, anti-CD33 humanized mAb, anti-CD52 humAb, anti-CD1 mAb (ior t6), MDX-22, celogovab, anti-17-1A mAb, bevacizumab, daclizumab, anti-TAG-72 (MDX-220), anti-idiotypic mAb mimic of high molecular weight proteoglycan (I-Mel-1), anti-idiotypic mAb mimic of high molecular weight proteoglycan (I-Mel-2), anti-CEA Ab, hmAbH11, anti-DNA or DNA-associated proteins (histones) mAb, Gliomab-H mAb, GNI-250 mAb, anti-CD22, CMA 676), anti-idiotypic human mAb to GD2 ganglioside, ior egf/r3, anti-ior c2 glycoprotein mAb, ior c5, anti-FLK2/FLT-3 mAb, anti-GD-2 bispecific mAb, antinuclear autoantibodies, anti-HLA-DR Ab, anti-CEA mAb, palivizumab, bevacizumab, alemtuzumab, BLyS-mAb, anti-VEGF2, anti-Trail receptor; B3 mAb, mAb BR96, breast cancer; and Abx-Cbl mAb.

205. The method of claim 192 or 198, further comprising administering to the subject an antigen.

206. The method of claim 205, wherein the antigen is a cancer antigen or a microbial antigen.

207. The method of claim 206, wherein the microbial antigen is selected from the group consisting of a bacterial antigen, a mycobacterial antigen, a viral antigen, a fungal antigen, and a parasitic antigen.

208. The method of claim 207, wherein the bacterial antigen is derived from a bacterial species selected from the group consisting of E. coli, Staphylococcal, Streptococcal, Pseudomonas, Clostridium difficile, Legionella, Pneumococcus, Haemophilus, Klebsiella, Enterobacter, Citrobacter, Neisseria, Shigella, Salmonella, Listeria, Pasteurella, Streptobacillus, Spirillum, Treponema, Actinomyces, Borrelia, Corynebacterium, Nocardia, Gardnerella, Campylobacter, Spirochaeta, Proteus, Bacteriodes, H. pylori, and anthrax.

209. The method of claim 207, wherein the viral antigen is derived from a viral species selected from the group consisting of HIV, Herpes simplex virus 1, Herpes simplex virus 2, cytomegalovirus, hepatitis A virus, hepatitis B virus, hepatitis C virus, human papilloma virus, Epstein Barr virus, rotavirus, adenovirus, influenza A virus, respiratory syncytial virus, varicella-zoster virus, small pox, monkey pox and SARS.

210. The method of claim 207, wherein the fungal antigen is derived from a fungal species that causes an infection selected from the group consisting of candidiasis, ringworm, histoplasmosis, blastomycosis, paracoccidioidomycosis, crytococcosis, aspergillosis, chromomycosis, mycetoma infections, pseudallescheriasis, and tinea versicolor infection.

211. The method of claim 207, wherein the parasitic antigen is derived from a parasite species selected from the group consisting of amebiasis, Trypanosoma cruzi, Fascioliasis, Leishmaniasis, Plasmodium, Onchocerciasis, Paragonimiasis, Trypanosoma brucei, Pneumocystis, Trichomonas vaginalis, Taenia, Hymenolepsis, Echinococcus, Schistosomiasis, neurocysticercosis, Necator americanus, and Trichuris trichuria.

212. The method of claim 206, wherein the cancer antigen is selected from the group consisting of MART-1/Melan-A, gp100, adenosine deaminase-binding protein (ADAbp), FAP, cyclophilin b, colorectal associated antigen (CRC)—C017-1A/GA733, carcinoembryonic antigen (CEA), CAP-1, CAP-2, etv6, AML1, prostate specific antigen (PSA), PSA-1, PSA-2, PSA-3, prostate-specific membrane antigen (PSMA), T-cell receptor/CD3-zeta chain, HER 2, CD33, EGF receptor, HLA markers such as HLA-DR, CD52, CD1, CEA, CD22, GD2 ganglioside, FLK2/FLT3, VEGF, VEGFR and CD20.

213. The method of claim 206, wherein the cancer antigen is selected from the group consisting of MAGE-AL, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-C5).

214. The method of claim 206, wherein the cancer antigen is selected from the group consisting of GAGE-1, GAGE-2, GAGE-3, GAGE-4, GAGE-5, GAGE-6, GAGE-7, GAGE-8, GAGE-9.

215. The method of claim 206, wherein the cancer antigen is selected from the group consisting of BAGE, RAGE, LAGE-1, NAG, GnT-V, MUM-1, CDK4, tyrosinase, p53, MUC family, HER2/neu, p21ras, RCAS1, α-fetoprotein, E-cadherin, α-catenin, β-catenin, γ-catenin, p120ctn, gp100^{Pmel117}, PRAME, NY-ESO-1, cdc27, adenomatous polyposis coli protein (APC), fodrin, Connexin 37, Ig-idiotype, p15, gp75, GM2 ganglioside, GD2 ganglioside, human papilloma virus proteins, Smad family of tumor antigens, Imp-1, P1A, EBV-encoded nuclear antigen (EBNA)-1, brain glycogen phosphorylase, SSX-1, SSX-2 (HOM-MEL-40), SSX-1, SSX-4, SSX-5, SCP-1 and CT-7, and c-erbB-2.

216. The method of claim 192 or 198, wherein the subject will have a surgery.

217. The method of claim 192 or 198, wherein the subject has a skin abrasion from a trauma.

218. The method of claim 192 or 198, wherein the subject is traveling to a region in which a microbial infection is common.

219. The method of claim 205, wherein the agent of Formula I and the antigen are formulated together.

220. The method of claim 205, wherein the antigen is administered mucosally.

221. The method of claim 198, wherein the subject has been treated with an agent selected from the group consisting of a cox-1 inhibitor, a cox-2 inhibitor, and a steroid.

222. The method of claim 221, wherein the agent is celecoxib, rofecoxib, naproxen or aspirin.

223. The method of claim 221, wherein the subject is a substance abuse subject.

224. The method of claim 223, wherein the substance is selected from the group consisting of alcohol and intravenous drug.

225. The method of claim 198, wherein the subject has gingivitis, osteomyelitis, diabetes type I, diabetes type II, chronic granuloma, chronic hepatitis, and chronic EBV infection.

226. The method of claim 194 or 203, wherein the antibody or antibody fragment is an antibody or antibody fragment specific for a cell surface molecule.

227. The method of claim 226, wherein the cell surface molecule is selected from the group consisting of HER 2, CD20, CD33, EGF receptor, HLA markers such as HLA-DR, CD52, CD1, CEA, CD22, GD2 ganglioside, FLK2/FLT3, VEGF, VEGFR.

228. The method of claim 194 or 203, wherein the antibody or antibody fragment is an antibody or antibody fragment specific for a cancer antigen.

229. The method of claim 228, wherein the cancer antigen is selected from the group consisting of HER 2 (p185), CD20, CD33, GD3 ganglioside, GD2 ganglioside, carcinoembryonic antigen (CEA), CD22, milk mucin core protein, TAG-72, Lewis A antigen, ovarian associated antigens such as OV-TL3 and MOv18, high Mr melanoma antigens recognized by antibody 9.2.27, HMFG-2, SM-3, B72.3, PR5C5, and PR4D2.

230. The method of claim 194 or 203, wherein the antibody or antibody fragment is an antibody or antibody fragment specific for a stromal cell molecule.

231. The method of claim 230, wherein the stromal cell molecule is selected from the group consisting of FAP and CD26.

232. The method of claim 194 or 203, wherein the antibody or antibody fragment is an antibody or antibody fragment specific for an extracellular matrix molecule.

233. The method of claim 230, wherein the extracellular matrix molecule is selected from the group consisting of collagen, glycosaminoglycans (GAGs), proteoglycans, elastin, fibronectin and laminin.

234. The method of claim 194 or 203, wherein the antibody or antibody fragment is an antibody or antibody fragment specific for a tumor vasculature associated antigen.

235. The method of claim 234, wherein the tumor vasculature associated antigen is selected from the group consisting of endoglin, ELAM-1, VCAM-1, ICAM-1, ligand reactive with LAM-1, MHC class II antigens, aminophospholipids such as phosphatidylserine and phosphatidylethanolamine, VEGFR1 (Flt-1), VEGFR2 (KDR/Flk-1), and a complex of a growth factor and its receptor such as a complex of FGF and the FGFR or a complex of TGFβ and the TGFβR.

236. The method of claim 234, wherein the antibody or antibody specific for a tumor vasculature associated antigen is selected from the group consisting of TEC-4 and TEC-11, 2C3 (ATCC PTA 1595), GV39 and GV97.

237. The method of claim 194 or 203, wherein the antibody or antibody fragment is administered on a first day of a seven day cycle and the agent of Formula I is administered twice a day on day two through day seven.

238. The method of claim 237, wherein the seven day cycle is repeated twice, thrice, or four times.

239. The method of claim 237, wherein the seven day cycle is repeated for a month, two months, or three months.

240. The method of claim 207, wherein the mycobacterial antigen is derived from a mycobacterial species selected from the group consisting of M. tuberculosis and M. leprae.

241. The method of claim 194 or 203, wherein the antibody or antibody fragment is conjugated to a toxin derived from plant, fungus, or bacteria.

242. The method of claim 241, wherein the toxin is selected from the group consisting of A chain toxin, deglycosylated A chain toxin, ribosome inactivating protein, α-sarcin, aspergillin, restrictocin, ribonuclease, diptheria toxin and Pseudomonas exotoxin.

243. The method of claim 203, wherein the antibody or antibody fragment is conjugated to a chemotherapeutic agent or a radioisotope.

244. The method of claim 243, wherein the chemotherapeutic agent is selected from the group consisting of an anti-metabolite, an anthracycline, a vinca alkaloid, an antibiotic, an alkylating agent, and an epipodophyllotoxin.

245. The method of claim 192, 194, 198 or 203, wherein the cancer antigen is a gene or gene product thereof that has undergone chromosomal alteration.

246. The method of claim 245, wherein the gene product is an RNA or protein gene product.

247. The method of claim 245, wherein the gene or gene product that has undergone chromosomal alteration is selected from the group consisting of gene or gene products associated with activation of quiescent genes, and gene or gene products associated with a novel fusion gene and protein.

248. The method of claim 247, wherein the gene or gene products associated with activation of quiescent genes is selected from the group consisting of BCL-1 and IgH, BCL-2 and IgH, BCL-6, TAL-1 and TCRδ or SIL, c-MYC and IgH or IgL, MUN/IRF4 and IgH, and PAX-5 (BSAP).

249. The method of claim 247, wherein the gene or gene products associated with a novel fusion gene and protein is selected from the group consisting of RARα, PML, PLZF, NPM or NuMA; BCR and ABL; MLL (HRX); E2A and PBX or HLF; NPM, ALK; and NPM, MLF-1.

250. The method of claim 192, 194, 198 or 203, wherein the cancer antigen is a tissue- or lineage-specific antigen.

251. The method of claim 250, wherein the tissue- or lineage-specific antigen is a cell surface protein, epidermal growth factor receptor, cell-associated protein, or a secreted protein.

252. The method of claim 250, wherein the cell surface protein is selected from the group consisting of CD20, CD22, CD52, CD33, CD10 (gp100), CD3/T-cell receptor (TCR), CD79/B-cell receptor (BCR), CD26, Human leukocyte antigen (HLA)-DR, HLA-DP, and HLA-DQ, RCAS1, and Prostate specific membrane antigen.

253. The method of claim 250, wherein the epidermal growth factor receptor is selected from the group consisting of EGFR (HER1 or erbB1) and EGFRvIII, erbB2 (HER2 or HER2/neu), erbB3 (HER3), and erbB4 (HER4).

254. The method of claim 250, wherein the cell-associated protein is selected from the group consisting of Tyrosinase, Melan-A/MART-1, tyrosinase related protein (TRP)-1/gp75, Polymorphic epithelial mucin (PEM), and Human epithelial mucin (MUC1).

255. The method of claim 250, wherein the secreted protein is selected from the group consisting of monoclonal immunoglobulin, immunoglobulin light chains, α-fetoprotein, kallikreins 6 and 10, gastrin-releasing peptide/bombesin, and prostate specific antigen.

256. The method of claim 192, 194, 198 or 203, wherein the cancer antigen is a cancer testis (CT) antigen.

257. The method of claim 256, wherein the cancer testis (CT) antigen is selected from the group consisting of MAGE, MAGE-A1, -A3, -A6, -A12, MAGE-3, BAGE, GAGE, GAGE-1, -2, -3, -4, -5, -6, -7, and -8, HAGE, LAGE-1, NY-ESO-1, RAGE, RAGE-1, -2, -4, SSX, SSX-1, -2, -3, -4, -5, -6, -7, -8, -9, HOM-TES-14/SCP-1, HOM-TES-85, HOM-MEL-40, and PRAME.

258. The method of claim 192, 194, 198 or 203, wherein the cancer antigen is a non-tissue or non-lineage specific antigen.

259. The method of claim 258, wherein the non-tissue or non-lineage specific antigen is a carcinoembryonic antigen family member.

260. The method of claim 259, wherein the carcinoembryonic antigen family member is selected from the group consisting of CD66a, CD66b, CD66c, CD66d and CD66e.

261. The method of claim 192, 194, 198 or 203, wherein the cancer antigen is a viral protein.

262. The method of claim 261, wherein the viral protein is selected from the group consisting of Human papilloma virus protein, and EBV-encoded nuclear antigen (EBNA)-1.

263. The method of claim 192, 194, 198 or 203, wherein the cancer antigen is an antigen that is mutated or aberrantly expressed in a cancer.

264. The method of claim 263, wherein the antigen that is mutated or aberrantly expressed in a cancer is CDK4 or beta-catenin.

265. The method of claim 194 or 203, wherein the antibody or antibody fragment is selected from the group consisting of Avastin (bevacizumab), BEC2 (mitumomab), Bexxar (tositumomab), Campath (alemtuzumab), CeaVac, Herceptin (trastuzumab), IMC-C225 (centuximab), LymphoCide (epratuzumab), MDX-210, Mylotarg (gemtuzumab ozogamicin), Panorex (edrecolomab), Rituxan (rituximab), Theragyn (pemtumomab), Zamyl, and Zevalin (ibritumomab tituxetan).

266. The method of claim 192, 194, 198 or 203, wherein the cancer antigen is selected from the group consisting of VEGF, Anti-idiotypic mAb (GD3 ganglioside mimic), CD20, CD52, Anti-idiotypic mAb (CEA mimic), ERBB2, EGFR, CD22, ERBB2 X CD65 (fcγRI), CD33, EpCam, and PEM.

267. A method for treating a subject having or at risk of developing an IFN-responsive condition comprising
administering to a subject in need of such treatment an agent of Formula I in an amount effective to induce a therapeutically or prophylactically effective amount of IL-1 in the subject,
wherein the agent of Formula I is administered by injection or in an enterically coated form.

268. The method of claim 267, wherein the IFN-responsive condition is a chronic infection selected from the group consisting of a chronic hepatitis B infection, chronic hepatitis C infection, chronic Epstein Barr Virus infection, and tuberculosis.

269. The method of claim 268, further comprising administering a second active agent selected from the group consisting of IFNα, pegylated IFN, IFNα-2b, acyclovir, lobucavir, ganciclovir, L-deoxythymidine, clevudine, a therapeutic vaccine, phosphonoformate (PFA), ribavirin (RBV), thymosin alpha-1, 2 3-dideoxy-3-fluoroguanosine (FLG), famciclovir, lamivudine, adefovir dipivoxil, entecavir, emtricitabine, and hepatitis B-specific immunoglobulin.

270. The method of claim 268, wherein the subject is HIV positive.

271. The method of claim 267, wherein the disorder has become drug resistant.

272. The method of claim 267, wherein the disorder is multiple sclerosis.

273. The method of claim 267, wherein IFN is selected from the group consisting of IFNα, IFNα-2b, IFNβ, IFN-γ.

274. The method of claim 267, wherein the IFN-responsive condition is an IFN-γ responsive condition.

275. The method of claim 274, wherein the IFN-γ responsive condition is selected from the group consisting of viral infections and associated diseases, and cancer.

276. A method for treating a subject having or at risk of developing cancer comprising
administering to a subject in need of such treatment an enzyme inhibitor selected from the group consisting of a tyrosine kinase inhibitor, a CDK inhibitor, a MAP kinase inhibitor, and an EGFR inhibitor, and an agent of Formula I in an amount effective to inhibit the cancer,
wherein the agent of Formula I is administered by injection or in an enterically coated form.

277. The method of claim 276, wherein the amount effective is a synergistic amount.

278. The method of claim 276, wherein the CDK inhibitor is selected from the group consisting of p21, p27, p57, p15, p16, p18, and p19.

279. The method of claim 276, wherein the MAP kinase inhibitor is selected from the group consisting of KY12420 (C₂₃H₂₄O₈), CNI-1493, PD98059, 4-(4-Fluorophenyl)-2-(4-methylsulfinyl phenyl)-5-(4-pyridyl) 1H-imidazole.

280. The method of claim 276, wherein the EGFR inhibitor is selected from the group consisting of Tarceva™ (OSI-774), Iressa (ZD1839), WHI-P97 (quinazoline derivative), LFM-A12 (leflunomide metabolite analog), AG1458.

281. A method for treating a subject having or at risk of developing cardiovascular disease comprising
administering to a subject in need of such treatment an agent of Formula I in an amount effective to induce an effective amount of IL-1.

282. The method of claim 280, further comprising identifying the subject in need of such treatment.

283. A method for preventing drug resistance in a subject having an infectious disease comprising
administering to a subject receiving an anti-microbial agent, an agent of Formula I in an amount effective to reduce the risk of resistance to the anti-microbial agent,
wherein the agent of Formula I is administered by injection or in an enterically coated form.

284. The method of claim 283, wherein the infectious disease is selected from the group consisting of a bacterial infection, a mycobacterial infection, a viral infection, a fungal infection and a parasitic infection.

285. The method of claim 283, wherein the bacterial infection is a Pseudomonas infection.

286. The method of claim 283, wherein the anti-microbial agent is selected from the group consisting of an anti-bacterial agent, an anti-mycobacterial agent, an anti-viral agent, an anti-fungal agent, and an anti-parasitic agent.

287. A method of shortening a vaccination course comprising
administering to a subject in need of immunization an agent of Formula I in an amount effective to induce an antigen-specific immune response to a vaccine administered in a vaccination course,
wherein the vaccination course is shortened by at least one immunization,
wherein the agent of Formula I is administered by injection or in an enterically coated form.

288. The method of claim 287, wherein the vaccine is for hepatitis virus.

289 The method of claim 288, wherein hepatitis is hepatitis B virus.

290. A method of shortening a vaccination course comprising
administering to a subject in need of immunization an agent of Formula I in an amount effective to induce an antigen-specific immune response to a vaccine administered in a vaccination course,
wherein the vaccination course is shortened by at least one day,
wherein the agent of Formula I is administered by injection or in an enterically coated form.

291. The method of claim 287 or 290, wherein the agent of Formula I is administered substantially simultaneously with the vaccine.

292. The method of claim 290, wherein the vaccine is for hepatitis virus.

293. The method of claim 292, wherein hepatitis virus is hepatitis B virus.

294. A method for stimulating an immune response in a subject having cancer comprising
administering to a subject in need of such treatment an agent of Formula I in an amount effective to stimulate an antigen-specific immune response, prior to and following a therapy selected from the group consisting of radiation, surgery and chemotherapy,
wherein the agent of Formula I is administered by injection or in an enterically coated form.

295. The method of claim 294, further comprising administering an adjuvant to the subject.

296. The method of claim 295, wherein the adjuvant is selected from the group consisting of alum, cholera toxin, CpG immunostimulatory nucleic acids, MPL, MPD, and QS-21.

297. The method of claim 294, wherein the agent of Formula I is administered to the subject 30 minutes to 8 hours before the therapy and 30 minutes to 8 hours after the therapy.

298. The method of claim 294, wherein the agent of Formula I is administered in a dose of greater than 10⁻⁸M.

299. A method for stimulating an immune response in a subject at risk of developing cancer comprising
administering to a subject in need of such treatment an agent of Formula I in an amount effective to stimulate an antigen-specific immune response,
wherein the agent of Formula I is administered by injection or in an enterically coated form.

300. The method of claim 299, further comprising identifying a subject in need of such treatment.

301. The method of claim 299, wherein the subject at risk of developing cancer has a familial predisposition to developing cancer.

302. The method of claim 301, wherein the familial predisposition is familial colon polyposis.

303. The method of claim 299, wherein the subject has precancerous polyps.

304. The method of claim 299, wherein the subject has precancerous HPV lesions.

305. The method of claim 299, wherein the subject is at risk of developing a cancer that is a metastasis.

306. The method of claim 299, further comprising administering an adjuvant to the subject.

307. The method of claim 306, wherein the adjuvant is selected from the group consisting of alum, cholera toxin, CpG immunostimulatory nucleic acids, MPL, MPD, and QS-21.

308. The method of claim 299, wherein the agent of Formula I is administered in a dose of greater than 10⁻⁸M.

309. A method for modulating an immune response comprising
administering to a subject in need thereof an antibody or an antibody fragment on a first day of a seven day cycle, and administering to the subject an agent of Formula I on day 2 through to day 7 of the seven day cycle,
wherein the agent of Formula I is administered by injection or in an enterically coated form.

310. The method of claim 309, wherein the agent is administered twice a day on day 2 through to day 7.

311. The method of claim 309, wherein the seven day cycle is repeated twice, thrice, or four times.

312. The method of claim 309, wherein the seven day cycle is repeated for a month or two months.

313. The method of claim 309, wherein the antibody or antibody fragment is an antibody or antibody fragment specific for a cell surface molecule.

314. The method of claim 313, wherein the cell surface molecule is selected from the group consisting of HER 2, CD20, CD33, EGF receptor, HLA markers such as HLA-DR, CD52, CD1, CEA, CD22, GD2 ganglioside, FLK2/FLT3, VEGF, VEGFR.

315. The method of claim 309, wherein the antibody or antibody fragment is an antibody or antibody fragment specific for a cancer antigen.

316. The method of claim 315, wherein the cancer antigen is selected from the group consisting of HER 2 (p185), CD20, CD33, GD3 ganglioside, GD2 ganglioside, carcinoembryonic antigen (CEA), CD22, milk mucin core protein, TAG-72, Lewis A antigen, ovarian associated antigens such as OV-TL3 and MOv18, high Mr melanoma antigens recognized by antibody 9.2.27, HMFG-2, SM-3, B72.3, PR5C5, PR4D2.

317. The method of claim 309, wherein the antibody or antibody fragment is an antibody or antibody fragment specific for a stromal cell molecule.

318. The method of claim 317, wherein the stromal cell molecule is selected from the group consisting of FAP and CD26.

319. The method of claim 309, wherein the antibody or antibody fragment is an antibody or antibody fragment specific for an extracellular matrix molecule.

320. The method of claim 578, wherein the extracellular matrix molecule is selected from the group consisting of collagen, glycosaminoglycans (GAGs), proteoglycans, elastin, fibronectin and laminin.

321. The method of claim 315, wherein the antibody or antibody fragment is an antibody or antibody fragment specific for a tumor vasculature associated antigen.

322. The method of claim 321, wherein the tumor vasculature associated antigen is selected from the group consisting of endoglin, ELAM-1, VCAM-1, ICAM-1, ligand reactive with LAM-I, MHC class II antigens, aminophospholipids such as phosphatidylserine and phosphatidylethanolamine, VEGFR1 (Flt-1), VEGFR2 (KDR/Flk-1), a complex of a growth factor and its receptor such as a complex of FGF and the FGFR or a complex of TGFβ and the TGFβR.

323. The method of claim 321, wherein the antibody or antibody specific for a tumor vasculature associated antigen is selected from the group consisting of TEC-4 and TEC-11, 2C3 (ATCC PTA 1595), GV39 and GV97.

324. The method of claim 315, wherein the antibody or antibody fragment is conjugated to a toxin derived from plant, fungus, or bacteria.

325. The method of claim 324, wherein the toxin is selected from the group consisting of A chain toxin, deglycosylated A chain toxin, ribosome inactivating protein, α-sarcin, aspergillin, restrictocin, ribonuclease, diptheria toxin and Pseudomonas exotoxin.

326. The method of claim 315, wherein the antibody or antibody fragment is conjugated to a chemotherapeutic agent, a radioisotope or a cytotoxin.

327. The method of claim 326, wherein the chemotherapeutic agent is selected from the group consisting of an anti-metabolite, an anthracycline, a vinca alkaloid, an antibiotic, an alkylating agent, and an epipodophyllotoxin.

328. The method of claim 206 or 315, wherein the cancer antigen is a gene or gene product thereof that has undergone chromosomal alteration.

329. The method of claim 328, wherein the gene product is an RNA or protein gene product.

330. The method of claim 328, wherein the gene or gene product that has undergone chromosomal alteration is selected from the group consisting of gene or gene products associated with activation of quiescent genes, and gene or gene products associated with a novel fusion gene and protein.

331. The method of claim 330, wherein the gene or gene products associated with activation of quiescent genes is selected from the group consisting of BCL-1 and IgH, BCL-2 and IgH, BCL-6, TAL-1 and TCRδ or SIL, c-MYC and IgH or IgL, MUN/IRF4 and IgH, and PAX-5 (BSAP).

332. The method of claim 330, wherein the gene or gene products associated with a novel fusion gene and protein is selected from the group consisting of RARα, PML, PLZF, NPM or NuMA, BCR and ABL, MLL (HRX), E2A and PBX or HLF, NPM, ALK, and NPM, MLF-1.

333. The method of claim 206 or 315, wherein the cancer antigen is a tissue- or lineage-specific antigen.

334. The method of claim 33, wherein the tissue- or lineage-specific antigen is a cell surface protein, epidermal growth factor receptor, cell-associated protein, or a secreted protein.

335. The method of claim 334, wherein the cell surface protein is selected from the group consisting of CD20, CD22, CD52, CD33, CD10 (gp100), CD3/T-cell receptor (TCR), CD79/B-cell receptor (BCR), CD26, Human leukocyte antigen (HLA)-DR, HLA-DP, and HLA-DQ, RCAS1, and Prostate specific membrane antigen.

336. The method of claim 334, wherein the epidermal growth factor receptor is selected from the group consisting of EGFR (HER1 or erbB1) and EGFRvIII, erbB2 (HER2 or HER2/neu), erbB3 (HER3), and erbB4 (HER4).

337. The method of claim 334, wherein the cell-associated protein is selected from the group consisting of Tyrosinase, Melan-A/MART-1, tyrosinase related protein (TRP)-1/gp75, Polymorphic epithelial mucin (PEM), and Human epithelial mucin (MUC1).

338. The method of claim 334, wherein the secreted protein is selected from the group consisting of Monoclonal immunoglobulin, Immunoglobulin light chains, α-fetoprotein, Kallikreins 6 and 10, Gastrin-releasing peptide/bombesin, and Prostate specific antigen.

339. The method of claim 206 or 315, wherein the cancer antigen is a cancer testis (CT) antigen.

340. The method of claim 339, wherein the cancer testis (CT) antigen is selected from the group consisting of MAGE, MAGE-A1, -A3, -A6, -A12, MAGE-3, BAGE, GAGE, GAGE-1, -2, -3, -4, -5, -6, -7, and -8, HAGE, LAGE-1, NY-ESO-1, RAGE, RAGE-1, -2, -4, SSX, SSX-1, -2, -3, -4, -5, -6, -7, -8, -9, HOM-TES-14/SCP-1, HOM-TES-85, HOM-MEL-40, and PRAME.

341. The method of claim 206 or 315, wherein the cancer antigen is a non-tissue or non-lineage specific antigen.

342. The method of claim 341, wherein the non-tissue or non-lineage specific antigen is a carcinoembryonic antigen family member.

343. The method of claim 342, wherein the carcinoembryonic antigen family member is selected from the group consisting of CD66a, CD66b, CD66c, CD66d and CD66e.

344. The method of claim 206 or 315, wherein the cancer antigen is a viral protein.

345. The method of claim 344, wherein the viral protein is selected from the group consisting of Human papilloma virus protein and EBV-encoded nuclear antigen (EBNA)-1.

346. The method of claim 206 or 315, wherein the cancer antigen is an antigen that is mutated or aberrantly expressed in a cancer.

347. The method of claim 346, wherein the antigen that is mutated or aberrantly expressed in a cancer is CDK4 or beta-catenin.

348. The method of claim 206 or 315, wherein the cancer antigen is selected from the group consisting of VEGF, Anti-idiotypic mAb (GD3 ganglioside mimic), CD20, CD52, Anti-idiotypic mAb (CEA mimic), ERBB2, EGFR, CD22, ERBB2 X CD65 (fcγRI), CD33, EpCam, and PEM.

349. The method of claim 2, 172, 294, or 299, wherein the cancer is selected from the group consisting of basal cell carcinoma, biliary tract cancer; bladder cancer; bone cancer; brain cancer; breast cancer; cervical cancer; choriocarcinoma; CNS cancer; colon and rectum cancer; connective tissue cancer; cancer of the digestive system; endometrial cancer; esophageal cancer; eye cancer; cancer of the head and neck; gastric cancer; intra-epithelial neoplasm; kidney cancer; larynx cancer; leukemia; chronic myeloid leukemia, chronic lymphoid leukemia, acute myeloid leukemia, acute lymphoid leukemia, liver cancer; small cell lung cancer; non-small cell lung cancer; lymphoma, Hodgkin's lymphoma; Non-Hodgkin's lymphoma; melanoma; myeloma; neuroblastoma; oral cavity cancer; ovarian cancer; pancreatic cancer; prostate cancer; retinoblastoma; rhabdomyosarcoma; rectal cancer; renal cancer; cancer of the respiratory system; sarcoma; skin cancer; stomach cancer; testicular cancer; thyroid cancer; uterine cancer; and cancer of the urinary system.

350. The method of claim 2, 172, 294, or 299, wherein the cancer is selected from the group consisting of a lymphoma or leukemia.

351. The method of claim 2, 172, 294, or 299, wherein the cancer is selected from the group consisting of bladder cancer, breast cancer, colon cancer, endometrial cancer, head and neck cancer, leukemia, lung cancer, lymphoma, melanoma, ovarian cancer, prostate cancer and rectal cancer.

352. The method of claim 2, 172, 294, or 299, wherein the cancer is a refractory cancer.

353. The method of claim 2, 172, 294, or 299, wherein the refractory cancer is melanoma, renal cell carcinoma, pancreatic cancer, colon cancer, hepatic cancer, lung cancer, Non-Hodgkin's lymphoma or leukemia.

354. The method of claim 2, 172, 294, or 299, wherein the cancer is a metastasis.

355. The method of claim 13, 164 or 174, wherein the infectious disease is a bacterial infection, a viral infection, a fungal infection, a parasitic infection or a mycobacterial infection.

356. The method of claim 14, wherein the bacterial infection is selected from the group consisting of an E. coli infection, a Staphylococcal infection, a Streptococcal infection, a Pseudomonas infection, Clostridium difficile infection, Legionella infection, Pneumococcus infection, Haemophilus infection, Klebsiella infection, Enterobacter infection, Citrobacter infection, Neisseria infection, Shigella infection, Salmonella infection, Listeria infection, Pasteurella infection, Streptobacillus infection, Spirillum infection, Treponema infection, Actinomyces infection, Borrelia infection, Corynebacterium infection, Nocardia infection, Gardnerella infection, Campylobacter infection, Spirochaeta infection, Proteus infection, Bacteriodes infection, H. pylori infection, and anthrax infection.

357. The method of claim 14, wherein the viral infection is selected from the group consisting of an HIV infection, a Herpes simplex virus 1 infection, a Herpes simplex virus 2 infection, cytomegalovirus infection, hepatitis A virus infection, hepatitis B virus infection, hepatitis C virus infection, human papilloma virus infection, Epstein Barr virus infection, rotavirus infection, adenovirus infection, influenza A virus infection, respiratory syncytial virus infection, varicella-zoster virus infections, small pox infection, SARS infection, or monkey pox infection.

358. The method of claim 14, wherein the fungal infection is selected from the group consisting of candidiasis, ringworm, histoplasmosis, blastomycosis, paracoccidioidomycosis, crytococcosis, aspergillosis, chromomycosis, mycetoma infections, pseudallescheriasis, and tinea versicolor infection.

359. The method of claim 14, wherein the parasitic infection is selected from the group consisting of amebiasis, Trypanosoma cruzi infection, Fascioliasis, Leishmaniasis, Plasmodium infections, Onchocerciasis, Paragonimiasis, Trypanosoma brucei infection, Pneumocystis infection, Trichomonas vaginal is infection, Taenia infection, Hymenolepsis infection, Echinococcus infections, Schistosomiasis, neurocysticercosis, Necator americanus infection, and Trichuris trichuria infection.

360. The method of claim 14, wherein the viral infection is selected from the group consisting of a Herpes simplex virus 1 infection, a Herpes simplex virus 2 infection, cytomegalovirus infection, hepatitis A virus infection, hepatitis B virus infection, hepatitis C virus infection, human papilloma virus infection, Epstein Barr virus infection, rotavirus infection, adenovirus infection, influenza A virus infection, respiratory syncytial virus infection, varicella-zoster virus infections, small pox infection, monkey pox infection and SARS infection.

361. The method of claim 14 wherein the mycobacterial infection is selected from the group consisting of tuberculosis and leprosy.

362. The method of claim 1, 13, 112, 129, 164, 172, 192, 198, 267, 276, 281, 283, 287, 290, 294, 299, 309 or 568, wherein the agent of Formula I is an agent of Formula II.

363. The method of claim 1, 13, 112, 129, 164, 172, 192, 198, 267, 276, 281, 283, 287, 290, 294, 299, 309 or 568 wherein the agent of Formula I is an agent of Formula III.

364. The method of claim 1, 13, 112, 129, 164, 172, 192, 198, 267, 276, 281, 283, 287, 290, 294, 299, 309 or 568, wherein the agent of Formula I is Ile-boroPro.

365. The method of claim 1, 13, 112, 129, 164, 172, 192, 198, 267, 276, 281, 283, 287, 290, 294, 299, 309 or 568, wherein injection is subcutaneous injection.

366. The method of claim 1, 13, 112, 129, 164, 172, 192, 198, 267, 276, 281, 283, 287, 290, 294, 299, 309 or 568, wherein injection is intravenous injection, intramuscular injection, intraperitoneal injection, or intra-tumor injection.

367. The method of claim 1, 13, 112, 129, 164, 172, 192, 198, 267, 276, 281, 283, 287, 290, 294, 299, 309 or 568, wherein the enterically coated form is a pill, a capsule or a tablet.

368. The method of claim 1, 13, 112, 129, 164, 172, 192, 198, 267, 276, 281, 283, 287, 290, 294, 299, 309 or 568, wherein the effective amount is about 0.005 mg/kg to less than 1.0 mg/kg body weight per day.

369. The method of claim 1, 13, 112, 129, 164, 172, 192, 198, 267, 276, 281, 283, 287, 290, 294, 299, 309 or 568, wherein the agent of Formula I is at least 96% pure L-isomer.

370. The method of claim 1, 13, 112, 129, 164, 172, 192, 198, 267, 276, 281, 283, 287, 290, 294, 299, 309 or 568, wherein the subject is experiencing nausea.

371. The method of claim 1, 13, 112, 129, 164, 172, 192, 198, 267, 276, 281, 283, 287, 290, 294, 299, 309 or 568, wherein the subject is intolerant of Val-boroPro.

372. The method of claim 1, 13, 112, 129, 164, 172, 192, 198, 267, 276, 281, 283, 287, 290, 294, 299, 309 or 568, wherein the subject is otherwise free of symptoms calling for hemopoietic stimulation.

373. The method of claim 1, 13, 112, 129, 164, 172, 192, 198, 267, 276, 281, 283, 287, 290, 294, 299, 309 or 568, wherein the subject has normal hemopoietic activity.

374. The method of claim 1, 13, 112, 129, 164, 172, 192, 198, 267, 276, 281, 283, 287, 290, 294, 299, 309 or 568, wherein the subject is HIV negative.

375. The method of claim 1, 13, 112, 129, 164, 172, 192, 198, 267, 276, 281, 283, 287, 290, 294, 299, 309 or 568, wherein the agent of Formula I is administered on a routine schedule.

376. The method of claim 1, 13, 112, 129, 164, 172, 192, 198, 267, 276, 281, 283, 287, 290, 294, 299, 309 or 568, wherein the subject is first administered proline boropro.

377. The method of claim 1, 13, 112, 129, 172, 276, 283, 287, 290, 294, 299 or 309, wherein the agent of Formula I is administered in an amount that increases lymphoid tissue levels of IL-1, G-CSF or IL-8.

378. The method of claim 1, 13, 112, 129, 172, 276, 283, 287, 290, 294, 299 or 309, wherein the agent of Formula I is administered in an amount that does not increase serum IL-1 levels.

379. The method of claim 377, wherein the IL-1 is IL-1a or IL-1β.

380. The method of claim 378, wherein the IL-1 is IL-1α or IL-1β.

381. The method of claim 164, 192, 198, 267 or 281, wherein the IL-] is IL-1α or IL-1β.

382. The method of claim 112, 194, 203 or 309, wherein the antibody or antibody fragment is an antibody or antibody fragment specific for a cell surface molecule.

383. The method of claim 382, wherein the cell surface molecule is selected from the group consisting of HER 2, CD20, CD33, EGF receptor, HLA markers such as HLA-DR, CD52, CD1, CEA, CD22, GD2 ganglioside, FLK2/FLT3, VEGF and VEGFR.

384. The method of claim 112, 194, 203 or 309, wherein the antibody or antibody fragment is an antibody or antibody fragment specific for a cancer antigen.

385. The method of claim 384, wherein the cancer antigen is selected from the group consisting of HER 2 (p185), CD20, CD33, GD3 ganglioside, GD2 ganglioside, carcinoembryonic antigen (CEA), CD22, milk mucin core protein, TAG-72, Lewis A antigen, ovarian associated antigens such as OV-TL3 and MOv18, high Mr melanoma antigens recognized by antibody 9.2.27, HMFG-2, SM-3, B72.3, PR5C5 and PR4D2.

386. The method of claim 112, 194, 203 or 309, wherein the antibody or antibody fragment is an antibody or antibody fragment specific for a stromal cell molecule.

387. The method of claim 386, wherein the stromal cell molecule is selected from the group consisting of FAP and CD26.

388. The method of claim 112, 194, 203 or 309, wherein the antibody or antibody fragment is an antibody or antibody fragment specific for an extracellular matrix molecule.

389. The method of claim 388, wherein the extracellular matrix molecule is selected from the group consisting of collagen, glycosaminoglycans (GAGs), proteoglycans, elastin, fibronectin and laminin.

390. The method of claim 112, 194, 203 or 309, wherein the antibody or antibody fragment is an antibody or antibody fragment specific for a tumor vasculature associated antigen.

391. The method of claim 390, wherein the tumor vasculature associated antigen is selected from the group consisting of endoglin, ELAM-1, VCAM-1, ICAM-1, ligand reactive with LAM-1, MHC class II antigens, aminophospholipids such as phosphatidylserine and phosphatidylethanolamine, VEGFR1 (Flt-1), VEGFR2 (KDR/Flk-1), and a complex of a growth factor and its receptor such as a complex of FGF and the FGFR or a complex of TGFβ and the TGFβR.

392. The method of claim 390, wherein the antibody or antibody specific for a tumor vasculature associated antigen is selected from the group consisting of TEC-4 and TEC-11, 2C3 (ATCC PTA 1595), GV39 and GV97.

393. The method of claim 112, 194, 203 or 309, wherein the antibody or antibody fragment is administered on a first day of a seven day cycle and the agent of Formula I is administered twice a day on day two through day seven.

394. The method of claim 393, wherein the seven day cycle is repeated twice, thrice, or four times.

395. The method of claim 393, wherein the seven day cycle is repeated for a month, two months, or three months.

396. The method of claim 112, 194, 203 or 309, wherein the antibody or antibody fragment is conjugated to a toxin derived from plant, fungus, or bacteria.

397. The method of claim 396, wherein the toxin is selected from the group consisting of A chain toxin, deglycosylated A chain toxin, ribosome inactivating protein, α-sarcin, aspergillin, restrictocin, ribonuclease, diptheria toxin and Pseudomonas exotoxin.

398. The method of claim 112, 194, 203 or 309, wherein the antibody or antibody fragment is conjugated to a chemotherapeutic agent or a radioisotope.

399. The method of claim 398, wherein the chemotherapeutic agent is selected from the group consisting of an anti-metabolite, an anthracycline, a vinca alkaloid, an antibiotic, an alkylating agent, and an epipodophyllotoxin.

400. The method of claim 112, 194, 203 or 309, wherein the antibody or antibody fragment is selected from the group consisting of Avastin (bevacizumab), BEC2 (mitumomab), Bexxar (tositumomab), Campath (alemtuzumab), CeaVac, Herceptin (trastuzumab), IMC-C225 (centuximab), LymphoCide (epratuzumab), MDX-210, Mylotarg (gemtuzumab ozogamicin), Panorex (edrecolomab), Rituxan (rituximab), Theragyn (pemtumomab), Zamyl, and Zevalin (ibritumomab tituxetan).

401. The method of claim 144, 178, 206 or 315, wherein the cancer antigen is selected from the group consisting of MART-1/Melan-A, gp100, adenosine deaminase-binding protein (ADAbp), FAP, cyclophilin b, colorectal associated antigen (CRC)—C017-1A/GA733, carcinoembryonic antigen (CEA), CAP-1, CAP-2, etv6, AML1, prostate specific antigen (PSA), PSA-1, PSA-2, PSA-3, prostate-specific membrane antigen (PSMA), T-cell receptor/CD3-zeta chain, and CD20.

402. The method of claim 144, 178, 206 or 315, wherein the cancer antigen is selected from the group consisting of MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-C5).

403. The method of claim 144, 178, 206 or 315, wherein the cancer antigen is selected from the group consisting of GAGE-1, GAGE-2, GAGE-3, GAGE-4, GAGE-5, GAGE-6, GAGE-7, GAGE-8, GAGE-9.

404. The method of claim 144, 178, 206 or 315, wherein the cancer antigen is selected from the group consisting of BAGE, RAGE, LAGE-1, NAG, GnT-V, MUM-1, CDK4, tyrosinase, p53, MUC family, HER2/neu, p21ras, RCAS1, α-fetoprotein, E-cadherin, α-catenin, β-catenin, γ-catenin, p120ctn, gp100^{Pmel117}, PRAME, NY-ESO-1, cdc27, adenomatous polyposis coli protein (APC), fodrin, Connexin 37, Ig-idiotype, p15, gp75, GM2 ganglioside, GD2 ganglioside, human papilloma virus proteins, Smad family of tumor antigens, Imp-1, P1A, EBV-encoded nuclear antigen (EBNA)-1, brain glycogen phosphorylase, SSX-1, SSX-2 (HOM-MEL-40), SSX-1, SSX-4, SSX-5, SCP-1 and CT-7, and c-erbB-2.

405. A composition comprising
an effective amount of an agent of Formula I and an antibody or antibody fragment, wherein the agent of Formula I is formulated for administration by injection or in an enterically coated form.

406. The composition of claim 405, further comprising a pharmaceutically acceptable carrier.

407. The composition of claim 405, wherein the effective amount is an amount to stimulate antibody dependent cell-mediated cytoxicity.

408. The composition of claim 405, wherein the effective amount is an amount to treat or prevent cancer.

409. The composition of claim 405, wherein the effective amount is an amount to treat or prevent an infectious disease.

410. The composition of claim 405, wherein the antibody or antibody fragment is an antibody.

411. The composition of claim 405, wherein the antibody or antibody fragment is selected from the group consisting of trastuzumab, alemtuzumab (B cell chronic lymphocytic leukemia), gemtuzumab ozogamicin (CD33+ acute myeloid leukemia), hP67.6 (CD33+ acute myeloid leukemia), infliximab (inflammatory bowel disease and rheumatoid arthritis), etanercept (rheumatoid arthritis), rituximab, tositumomab, MDX-210, oregovomab, anti-EGF receptor mAb, MDX-447, anti-tissue factor protein (TF), (Sunol); ior-c5, c5, edrecolomab, ibritumomab tiuxetan, anti-idiotypic mAb mimic of ganglioside GD3 epitope, anti-HLA-Dr10 mAb, anti-CD33 humanized mAb, anti-CD52 humAb, anti-CD1 mAb (ior t6), MDX-22, celogovab, anti-17-1A mAb, bevacizumab, daclizumab, anti-TAG-72 (MDX-220), anti-idiotypic mAb mimic of high molecular weight proteoglycan (I-Mel-]), anti-idiotypic mAb mimic of high molecular weight proteoglycan (I-Mel-2), anti-CEA Ab, hmAbH11, anti-DNA or DNA-associated proteins (histones) mAb, Gliomab-H mAb, GNI-250 mAb, anti-CD22, CMA 676), anti-idiotypic human mAb to GD2 ganglioside, ior egf/r3, anti-ior c2 glycoprotein mAb, ior c5, anti-FLK2/FLT-3 mAb, anti-GD-2 bispecific mAb, antinuclear autoantibodies, anti-HLA-DR Ab, anti-CEA mAb, palivizumab, bevacizumab, alemtuzumab, BLyS-mAb, anti-VEGF2, anti-Trail receptor; B3 mAb, mAb BR96, breast cancer; and Abx-Cbl mAb.

412. The composition of claim 405, wherein the antibody or antibody fragment is an anti-HER2 antibody.

413. The composition of claim 412, wherein the anti-HER2 antibody is trastuzumab.

414. The composition of claim 405, wherein the antibody or antibody fragment is an anti-CD20 antibody.

415. The composition of claim 414, wherein the anti-CD20 antibody is rituximab.

416. The composition of claim 405, wherein the antibody or antibody fragment is an antibody or antibody fragment specific for a cell surface molecule.

417. The composition of claim 416, wherein the cell surface molecule is selected from the group consisting of HER 2, CD20, CD33, EGF receptor, HLA markers such as HLA-DR, CD52, CD1, CEA, CD22, GD2 ganglioside, FLK2/FLT3, VEGF, and VEGFR.

418. The composition of claim 405, wherein the antibody or antibody fragment is an antibody or antibody fragment specific for a cancer antigen.

419. The composition of claim 418, wherein the cancer antigen is selected from the group consisting of HER 2 (p185), CD20, CD33, GD3 ganglioside, GD2 ganglioside, carcinoembryonic antigen (CEA), CD22, milk mucin core protein, TAG-72, Lewis A antigen, ovarian associated antigens such as OV-TL3 and MOv18, high Mr melanoma antigens recognized by antibody 9.2.27, HMFG-2, SM-3, B72.3, PR5C5, and PR4D2.

420. The composition of claim 405, wherein the antibody or antibody fragment is an antibody or antibody fragment specific for a stromal cell molecule.

421. The composition of claim 420, wherein the stromal cell molecule is selected from the group consisting of FAP and CD26.

422. The composition of claim 405, wherein the antibody or antibody fragment is an antibody or antibody fragment specific for an extracellular matrix molecule.

423. The composition of claim 422, wherein the extracellular matrix molecule is selected from the group consisting of collagen, glycosaminoglycans (GAGs), proteoglycans, elastin, fibronectin and laminin.

424. The composition of claim 405, wherein the antibody or antibody fragment is an antibody or antibody fragment specific for a tumor vasculature associated antigen.

425. The composition of claim 424, wherein the tumor vasculature associated antigen is selected from the group consisting of endoglin, ELAM-1, VCAM-1, ICAM-1, ligand reactive with LAM-1, MHC class II antigens, aminophospholipids such as phosphatidylserine and phosphatidylethanolamine, VEGFR1 (Flt-1), VEGFR2 (KDR/Flk-1), and a complex of a growth factor and its receptor such as a complex of FGF and the FGFR or a complex of TGFβ and the TGFβR.

426. The composition of claim 424, wherein the antibody or antibody specific for a tumor vasculature associated antigen is selected from the group consisting of TEC-4 and TEC-11, 2C3 (ATCC PTA 1595), GV39 and GV97.

427. The composition of claim 405, further comprising a housing and instructions for use.

428. The composition of claim 427, wherein the instructions for use indicate that the antibody or antibody fragment is administered on a first day of a seven day cycle and the agent of Formula I is administered twice a day on day two through day seven.

429. The composition of claim 428, wherein the seven day cycle is repeated twice, thrice, or four times.

430. The composition of claim 428, wherein the seven day cycle is repeated for a month, two months, or three months.

431. The composition of claim 405, wherein the antibody or antibody fragment is conjugated to a toxin derived from plant, fungus, or bacteria.

432. The composition of claim 431, wherein the toxin is selected from the group consisting of A chain toxin, deglycosylated A chain toxin, ribosome inactivating protein, α-sarcin, aspergillin, restrictocin, ribonuclease, diptheria toxin and Pseudomonas exotoxin.

433. The composition of claim 405, wherein the antibody or antibody fragment is conjugated to a chemotherapeutic agent or a radioisotope.

434. The composition of claim 433, wherein the chemotherapeutic agent is selected from the group consisting of an anti-metabolite, an anthracycline, a vinca alkaloid, an antibiotic, an alkylating agent, and an epipodophyllotoxin.

435. The composition of claim 405, wherein the antibody or antibody fragment is selected from the group consisting of Avastin (bevacizumab), BEC2 (mitumomab), Bexxar (tositumomab), Campath (alemtuzumab), CeaVac, Herceptin (trastuzumab), IMC-C225 (centuximab), LymphoCide (epratuzumab), MDX-210, Mylotarg (gemtuzumab ozogamicin), Panorex (edrecolomab), Rituxan (rituximab), Theragyn (pemtumomab), Zamyl, and Zevalin (ibritumomab tituxetan).

436. The composition of claim 418, wherein the cancer antigen is selected from the group consisting of VEGF, Anti-idiotypic mAb (GD3 ganglioside mimic), CD20, CD52, Anti-idiotypic mAb (CEA mimic), ERBB2, EGFR, CD22, ERBB2 X CD65 (fcγRI), CD33, EpCam, and PEM.

437. A composition comprising
an effective amount of an agent of Formula I and a cancer antigen,
wherein the agent of Formula I is formulated for administration by injection or in an enterically coated form.

438. The composition of claim 437, wherein the effective amount is an amount to treat or prevent cancer.

439. The composition of claim 437, wherein the cancer antigen is a peptide antigen.

440. The composition of claim 437, wherein the cancer antigen is a lipid antigen.

441. The composition of claim 437, wherein the cancer antigen is selected from the group consisting of MART-1/Melan-A, gp100, adenosine deaminase-binding protein (ADAbp), FAP, cyclophilin b, colorectal associated antigen (CRC)—C017-1A/GA733, carcinoembryonic antigen (CEA), CAP-1, CAP-2, etv6, AML1, prostate specific antigen (PSA), PSA-1, PSA-2, PSA-3, prostate-specific membrane antigen (PSMA), T-cell receptor/CD3-zeta chain, HER 2, CD33, EGF receptor, HLA markers such as HLA-DR, CD52, CD1, CEA, CD22, GD2 ganglioside, FLK2/FLT3, VEGF, VEGFR and CD20.

442. The composition of claim 437, wherein the cancer antigen is selected from the group consisting of MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-C5).

443. The composition of claim 437, wherein the cancer antigen is selected from the group consisting of GAGE-1, GAGE-2, GAGE-3, GAGE-4, GAGE-5, GAGE-6, GAGE-7, GAGE-8, GAGE-9.

444. The composition of claim 437, wherein the cancer antigen is selected from the group consisting of BAGE, RAGE, LAGE-1, NAG, GnT-V, MUM-1, CDK4, tyrosinase, p53, MUC family, HER2/neu, p21ras, RCAS1, α-fetoprotein, E-cadherin, α-catenin, β-catenin, γ-catenin, p120ctn, gp100^{Pmel117}, PRAME, NY-ESO-1, cdc27, adenomatous polyposis coli protein (APC), fodrin, Connexin 37, Ig-idiotype, p15, gp75, GM2 ganglioside, GD2 ganglioside, human papilloma virus proteins, Smad family of tumor antigens, Imp-1, P1A, EBV-encoded nuclear antigen (EBNA)-1, brain glycogen phosphorylase, SSX-1, SSX-2 (HOM-MEL-40), SSX-1, SSX-4, SSX-5, SCP-1 and CT-7, and c-erbB-2.

445. The composition of claim 437, wherein the agent of Formula I is formulated for administration at a dose of greater than 10⁻⁸M.

446. The composition of claim 418 or 437, wherein the cancer antigen is a gene or gene product thereof that has undergone chromosomal alteration.

447. The composition of claim 446, wherein the gene product is an RNA or protein gene product.

448. The composition of claim 446, wherein the gene or gene product thereof that has undergone chromosomal alteration is selected from the group consisting of gene or gene products associated with activation of quiescent genes, and gene or gene products associated with a novel fusion gene and protein.

449. The composition of claim 448, wherein the gene or gene products associated with activation of quiescent genes is selected from the group consisting of BCL-1 and IgH; BCL-2 and IgH; BCL-6, TAL-1 and TCRδ or SIL; c-MYC and IgH or IgL; MUN/IRF4 and IgH; and PAX-5 (BSAP)

450. The composition of claim 448, wherein the gene or gene products associated with a novel fusion gene and protein is selected from the group consisting of RARα, PML, PLZF, NPM or NuMA, BCR and ABL, MLL (HRX), E2A and PBX or HLF, NPM, ALK, and NPM, MLF-1.

451. The composition of claim 418 or 437, wherein the cancer antigen is a tissue- or lineage-specific antigen.

452. The composition of claim 451, wherein the tissue- or lineage-specific antigen is a cell surface protein, epidermal growth factor receptor, cell-associated protein, or a secreted protein.

453. The composition of claim 452, wherein the cell surface protein is selected from the group consisting of CD20, CD22, CD52, CD33, CD10 (gp100), CD3/T-cell receptor (TCR), CD79/B-cell receptor (BCR), CD26, Human leukocyte antigen (HLA)-DR, HLA-DP, and HLA-DQ, RCAS1, and Prostate specific membrane antigen.

454. The composition of claim 452, wherein the epidermal growth factor receptor is selected from the group consisting of EGFR (HER1 or erbB 1) and EGFRvIII, erbB2 (HER2 or HER2/neu), erbB3 (HER3), and erbB4 (HER4).

455. The composition of claim 452, wherein the cell-associated protein is selected from the group consisting of Tyrosinase, Melan-A/MART-1, tyrosinase related protein (TRP)-1/gp75, Polymorphic epithelial mucin (PEM), and Human epithelial mucin (MUC1).

456. The composition of claim 452, wherein the secreted protein is selected from the group consisting of Monoclonal immunoglobulin, Immunoglobulin light chains, α-fetoprotein, Kallikreins 6 and 10, Gastrin-releasing peptide/bombesin, and Prostate specific antigen.

457. The composition of claim 418 or 437, wherein the cancer antigen is a cancer testis (CT) antigen.

458. The composition of claim 298, wherein the cancer testis (CT) antigen is selected from the group consisting of MAGE, MAGE-A1, -A3, -A6, -A12, MAGE-3, BAGE, GAGE, GAGE-1, -2, -3, -4, -5, -6, -7, and -8, HAGE, LAGE-1, NY-ESO-1, RAGE, RAGE-1, -2, -4, SSX, SSX-1, -2, -3, -4, -5, -6, -7, -8, -9, HOM-TES-14/SCP-1, HOM-TES-85, HOM-MEL-40, and PRAME.

459. The composition of claim 418 or 437, wherein the cancer antigen is a non-tissue or non-lineage specific antigen.

460. The composition of claim 459, wherein the non-tissue or non-lineage specific antigen is a carcinoembryonic antigen family member.

461. The composition of claim 460, wherein the carcinoembryonic antigen family member is selected from the group consisting of CD66a, CD66b, CD66c, CD66d and CD66e.

462. The composition of claim 418 or 437, wherein the cancer antigen is a viral protein.

463. The composition of claim 462, wherein the viral protein is selected from the group consisting of Human papilloma virus protein and EBV-encoded nuclear antigen (EBNA)-1.

464. The composition of claim 418 or 437, wherein the cancer antigen is an antigen that is mutated or aberrantly expressed in a cancer.

465. The composition of claim 464, wherein the antigen that is mutated or aberrantly expressed in a cancer is CDK4 or beta-catenin.

466. The composition of claim 418 or 437, wherein the cancer antigen is selected from the group consisting of VEGF, Anti-idiotypic mAb (GD3 ganglioside mimic), CD20, CD52, Anti-idiotypic mAb (CEA mimic), ERBB2, EGFR, CD22, ERBB2 X CD65 (fcγRI), CD33, EpCam, and PEM.

467. A composition comprising
an effective amount of an agent of Formula I and a microbial antigen,
wherein the agent of Formula I is formulated for administration by injection or in an enterically coated form.

468. The composition of claim 467, wherein the effective amount is an amount to treat or prevent an infectious disease.

469. The composition of claim 467, wherein the microbial antigen is a peptide antigen.

470. The composition of claim 467, wherein the microbial antigen is a lipid antigen.

471. The composition of claim 467, wherein the microbial antigen is selected from the group consisting of a bacterial antigen, a mycobacterial antigen, a viral antigen, a fungal antigen, and a parasitic antigen.

472. The composition of claim 471, wherein the bacterial antigen is derived from a bacterial species selected from the group consisting of E. coli, Staphylococcal, Streptococcal, Pseudomonas, Clostridium difficile, Legionella, Pneumococcus, Haemophilus, Klebsiella, Enterobacter, Citrobacter, Neisseria, Shigella, Salmonella, Listeria, Pasteurella, Streptobacillus, Spirillum, Treponema, Actinomyces, Borrelia, Corynebacterium, Nocardia, Gardnerella, Campylobacter, Spirochaeta, Proteus, Bacteriodes, H. pylori, and anthrax.

473. The composition of claim 471, wherein the viral antigen is derived from a viral species selected from the group consisting of HIV, Herpes simplex virus 1, Herpes simplex virus 2, cytomegalovirus, hepatitis A virus, hepatitis B virus, hepatitis C virus, human papilloma virus, Epstein Barr virus, rotavirus, adenovirus, influenza A virus, respiratory syncytial virus, varicella-zoster virus, small pox, monkey pox, and SARS.

474. The composition of claim 471, wherein the fungal antigen is derived from a fungal species that causes an infection selected from the group consisting of candidiasis, ringworm, histoplasmosis, blastomycosis, paracoccidioidomycosis, crytococcosis, aspergillosis, chromomycosis, mycetoma infections, pseudallescheriasis, and tinea versicolor infection.

475. The composition of claim 471, wherein the parasitic antigen is derived from a parasite species selected from the group consisting of amebiasis, Trypanosoma cruzi, Fascioliasis, Leishmaniasis, Plasmodium, Onchocerciasis, Paragonimiasis, Trypanosoma brucei, Pneumocystis, Trichomonas vaginal is, Taenia, Hymenolepsis, Echinococcus, Schistosomiasis, neurocysticercosis, Necator americanus, and Trichuris trichuria.

476. The composition of claim 471, wherein the mycobacterial antigen is derived from a mycobacterial species selected from the group consisting of M. tuberculosis and M. leprae.

477. The composition of claim 405, 437 or 467, wherein the agent of Formula I is an agent of Formula II.

478. The composition of claim 405, 437 or 467, wherein the agent of Formula I is an agent of Formula III.

479. The composition of claim 405, 437 or 467, wherein the agent of Formula I is Ile-boroPro.

480. The composition of claim 405, 437 or 467, wherein injection is subcutaneous injection.

481. The composition of claim 405, 437 or 467, wherein injection is intravenous injection, intramuscular injection, intraperitoneal injection, intra-tumor injection.

482. The composition of claim 405, 437 or 467, wherein the enterically coated form is a pill, a capsule or a tablet.

483. The composition of claim 405, 437 or 467, wherein the effective amount is about 0.005 mg/kg to less than 1.0 mg/kg body weight per day.

484. The composition of claim 405, 437 or 467, wherein the agent of Formula I is at least 96% pure L-isomer.
